(19) United States
(12) Patent Application Publication
(10) Pub. No.: US 2020/0028082 A1
(43) Pub. Date: Jan. 23, 2020

(54) ORGANIC ELECTROLUMINESCENCE DEVICE

(71) Applicant: ROHM AND HAAS ELECTRONIC MATERIALS KOREA LTD, Cheonan-si, Chungcheongnam-do (KR)

(72) Inventors: Dong-Hyung LEE, Hwaseong-si (KR); Tae-Jin LEE, Hwaseong-si (KR); Bitnari KIM, Hwaseong-si (KR)

(73) Assignee: Rohm and Haas Electronic Materials Korea Ltd., Cheonan-si, Chungcheongnam-do (KR)

(21) Appl. No.: 16/335,839

(22) PCT Filed: Oct. 13, 2017
(86) PCT No.: PCT/KR2017/011300

§ 371 Date: Mar. 22, 2019

(30) Foreign Application Priority Data
Oct. 14, 2016 (KR) 10-2016-0133600
Oct. 11, 2017 (KR) 10-2017-0129677

(51) Int. Cl. H01L 51/00; C07C 211/61; C09K 11/06; C07D 403/14

(52) U.S. Cl. CPC H01L 51/006; C07C 211/61; H01L 51/5056; C07D 403/14; H01L 51/0072; C09K 11/06

(57) ABSTRACT

The present disclosure relates to an organic electroluminescence device. The organic electroluminescence device of the present disclosure comprises a specific combination of a host compound and a hole transport material which can provide excellent lifespan characteristics.

### TECHNICAL FIELD

The present disclosure relates to an organic electroluminescence device.

### BACKGROUND ART

An electroluminescence device (EL device) is a self-light-emitting device with the advantages of providing a wider viewing angle, a greater contrast ratio, and a faster response time. The first organic EL device was developed by Eastman Kodak, 1987, by using a low-molecular aromatic diamine and an aluminum complex as materials for forming a light-emitting layer (see Appl. Phys. Lett. 51, 913, 1987).

An organic electroluminescence device (OLED) changes electric energy into light by the injection of a charge into an organic light-emitting material, and commonly comprises an anode, a cathode, and an intermediate layer formed between the two electrodes. The intermediate layer of the organic electroluminescence device may be composed of a hole injection layer, a hole transport layer, an electron blocking layer, a light-emitting layer, an electron buffer layer, a hole blocking layer, an electron transport layer, an electron injection layer, etc. The materials used in the intermediate layer can be classified into a hole injection material, a hole transport material, an electron blocking material, a light-emitting material, an electron buffer material, a hole blocking material, an electron transport material, an electron injection material, etc., depending on functions. In the organic electroluminescence device, holes from an anode and electrons from a cathode are injected into a light-emitting layer by electric voltage, and an exciton having high energy is produced by the recombination of the holes and electrons. The organic light-emitting compound moves into an excited state by the energy and emits light from energy when the organic light-emitting compound returns to the ground state from the excited state.

The selection of a compound contained in the hole transport layer, etc., is recognized as a means for improving device characteristics such as hole transport efficiency to the light-emitting layer, luminous efficiency, and lifespan. Also, the light-emitting material of the organic electroluminescence device is the most important factor for determining the luminous efficiency of the device so that the light-emitting material should have high quantum efficiency and high electron and hole mobility, and the formed light-emitting layer should be uniform and stable. Such a light-emitting material is divided into a blue, green or red light-emitting material depending on a luminescent color, and further there is a yellow or orange light-emitting material. The light-emitting material can be used by mixing a host and dopant in order to improve color purity, luminous efficiency, and stability. Generally, a device having excellent EL characteristics is a structure including a light-emitting layer made by doping a dopant with a host. When using such a dopant/host material system, the selection thereof is important since the host material has a significant effect on the efficiency and lifespan of the light-emitting device.

Japanese Patent Publication No. 3670707 and Korean Laid-Open Patent Publication No. 2013-0099098 disclose spirobifluorene substituted with a diarylamine as an organic electroluminescence compound including a hole transport material. Also, Korean Patent Publication No. 1477614 discloses, as a host material, a compound in which a benzene ring is fused to one of two carbazoles in a biscarbazole structure and a heteroaryl containing nitrogen is bonded to one of two nitrogen atoms.

However, the aforementioned documents do not specifically disclose that an organic electroluminescence device using spirobifluorene substituted with a diarylamine is used as a hole transport material and a compound in which a benzene ring is fused to one of two carbazoles in a biscarbazole structure and a heteroaryl containing nitrogen is bonded to one of two nitrogen atoms as a host material.

### DISCLOSURE

### Problems to be Solved

The objective of the present disclosure is to provide an organic electroluminescence device having long lifespan by comprising a specific combination of a hole transport material and a host material.

### Solution to Problems

As a result of conducting diligent studies to solve the aforementioned technical problems, the present inventors found that the aforementioned objective can be achieved by an organic electroluminescence device comprising a first electrode; a second electrode facing the first electrode; an intermediate layer between the first electrode and the second electrode; wherein the intermediate layer comprises at least one layer of a hole transporting band and at least one layer of a light-emitting layer; wherein at least one layer of the hole transporting band comprises a compound represented by the following Formula 1; wherein at least one layer of the light-emitting layer comprises at least one dopant compound and at least one host compound; and the at least one host compound comprises a compound represented by the following Formula 2:

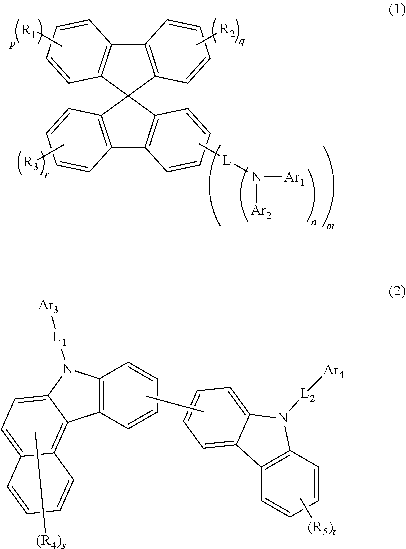

wherein the Formulae 1 and 2,

L, L₁ and L₂ each independently represent a single bond or a substituted or unsubstituted (C6-C30)arylene;

Ar₁ to Ar₄ each independently represent a substituted or unsubstituted (C6-C30)aryl or a substituted or unsubstituted (3- to 30-membered)heteroaryl;

R₁ to R₅ each independently represent hydrogen, deuterium, halogen, cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; or may be linked to an adjacent substituent to form a ring of a substituted or unsubstituted, (C3-C30) mono- or polycyclic, alicyclic or aromatic, or the combination thereof, whose carbon atom may be replaced with at least one heteroatom selected from nitrogen, oxygen, and sulfur;

m and n each independently represent an integer of 1 or 2;

p, q, r, and t each independently represent an integer of 1 to 4;

s represents an integer of 1 to 6;

when m, n, p, q, r, s or t represents an integer of 2 or more, each of [L-(NAr₁Ar₂)ₙ], each of (NAr₁Ar₂), each of R₁, each of R₂, each of R₃, each of R₄ or each of R₅ may be the same or different; and

the heteroaryl contains at least one heteroatom selected from B, N, O, S, Si, and P.

### Effects of the Invention

The present disclosure provides an organic electroluminescence device having long lifespan, and a display system or a lighting system can be produced by using the device.

### EMBODIMENTS OF THE INVENTION

Hereinafter, the present disclosure will be described in detail. However, the following description is intended to explain the invention, and is not meant in any way to restrict the scope of the invention.

The organic electroluminescence device of the present disclosure will be described in more detail as follows.

Herein, “(C1-C30)alkyl” is meant to be a linear or branched alkyl having 1 to 30 carbon atoms constituting the chain, in which the number of carbon atoms is preferably 1 to 10, more preferably 1 to 6, and includes methyl, ethyl, n-propyl, isopropyl, n-butyl, isobutyl, tert-butyl, etc. “(C2-C30)alkenyl” is meant to be a linear or branched alkenyl having 2 to 30 carbon atoms constituting the chain, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc. “(C2-C30)alkynyl” is a linear or branched alkynyl having 2 to 30 carbon atoms constituting the chain, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc. “(C3-C30)cycloalkyl” is a mono- or polycyclic hydrocarbon having 3 to 30 ring backbone carbon atoms, in which the number of carbon atoms is preferably 3 to 20, more preferably 3 to 7, and includes cyclopropyl, cyclobutyl, cyclopentyl, cyclohexyl, etc. “(3- to 7-membered)heterocycloalkyl” is a cycloalkyl having 3 to 7 ring backbone atoms and at least one heteroatom selected from the group consisting of B, N, O, S, Si, and P, preferably O, S, and N, and includes tetrahydrofuran, pyrrolidine, thiolan, tetrahydropyran, etc. “(C6-C30)aryl(ene)” is a monocyclic or fused ring radical derived from an aromatic hydrocarbon having 6 to 30 ring backbone carbon atoms and may be partially saturated, in which the number of ring backbone carbon atoms is preferably 6 to 20, more preferably 6 to 15, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc. “(3- to 30-membered)heteroaryl(ene)” is an aryl group having at least one, preferably 1 to 4 heteroatoms selected from the group consisting of B, N, O, S, Si, and P, and 3 to 30 ring backbone atoms, in which the number of ring backbone atoms is preferably 3 to 20, more preferably 5 to 15; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, di benzothiophenyl, benzonaphthothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. “(5- to 30-membered)heteroaryl(ene) containing nitrogen” is a heteroaryl group having at least one, preferably 1 to 4 heteroatoms selected from the group consisting of B, N, O, S, Si and P, in particular at least one N, and 5 to 30 ring backbone atoms, in which the number of ring backbone atoms is preferably 5 to 20, more preferably 5 to 15; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including pyrrolyl, imidazolyl, pyrazolyl, triazinyl, tetrazinyl, triazolyl, tetrazolyl, pyridyl, pyrazinyl, pyrimidinyl, pyridazinyl, etc., and a fused ring-type heteroaryl including benzimidazolyl, isoindolyl, indolyl, indazolyl, benzothiadiazolyl, quinolyl, isoquinolyl, cinnolinyl, quinazolinyl, quinoxalinyl, carbazolyl, phenanthridinyl, etc. “Halogen” includes F, Cl, Br, and I.

Herein, “substituted” in the expression “substituted or unsubstituted” means that a hydrogen atom is replaced with another atom or functional group (i.e., a substituent) in a certain functional group. The substituents of the substituted (C1-C30)alkyl, the substituted (C2-C30)alkenyl, the substituted (C2-C30)alkynyl, the substituted (C3-C30)cycloalkyl, the substituted (C6-C30)aryl(ene), the substituted (3- to 30-membered)heteroaryl, the substituted tri(C1-C30)alkylsilyl, the substituted tri(C6-C30)arylsilyl, the substituted di(C1-C30)alkyl(C6-C30)arylsilyl, the substituted (C1-C30)alkyldi(C6-C30)arylsilyl, the substituted mono- or di-(C1-C30)alkylamino, the substituted mono- or di-(C6-C30)arylamino, and the substituted alicyclic, aromatic, or combination thereof, of (C3-C30) mono- or polycyclic in L, L₁, L₂, Ar₁ to Ar₄, and R₁ to R₅ are each independently at least one selected from the group consisting of deuterium, halogen, cyano, carboxyl, nitro, hydroxyl, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, a (C6-C30)aryl-substituted or unsubstituted (5- to 30-membered)heteroaryl, a (5- to 30-membered)heteroaryl-substituted or unsubstituted (C6-C30)aryl , tri(C1-C30)alkylsilyl, tri(C6-C30)arylsilyl, di(C1-C30)alkyl(C6-C30)arylsilyl, (C1-C30)alkyldi(C6-C30)arylsilyl, amino, a mono- or di-(C1-C30)alkylamino, a (C1-C30)alkyl-substituted or unsubstituted mono- or di-(C6-C30)arylamino, (C1-C30)alkyl(C6-C30)arylamino, (C1-C30)alkylcarbonyl, (C1-C30)alkoxycarbonyl, (C6-C30)arylcarbonyl, di(C6-C30)arylboronyl, di(C1-C30)alkylboronyl, (C1-C30)alkyl(C6-C30)arylboronyl, (C6-C30)aryl(C1-C30)alkyl, and (C1-C30)alkyl(C6-C30)aryl, preferably, (C1-C6)alkyl, a (C6-C12)aryl-substituted or unsubstituted (5- to 20-membered)heteroaryl, a (5- to 20-membered)heteroaryl-substituted or unsubstituted (C6-C20)aryl, and (C1-C6)alkyl(C6-C20)aryl.

According to one embodiment of the organic electroluminescence device of the present disclosure, Formula 1 may be represented by any one of the following Formulae 3 to 5:

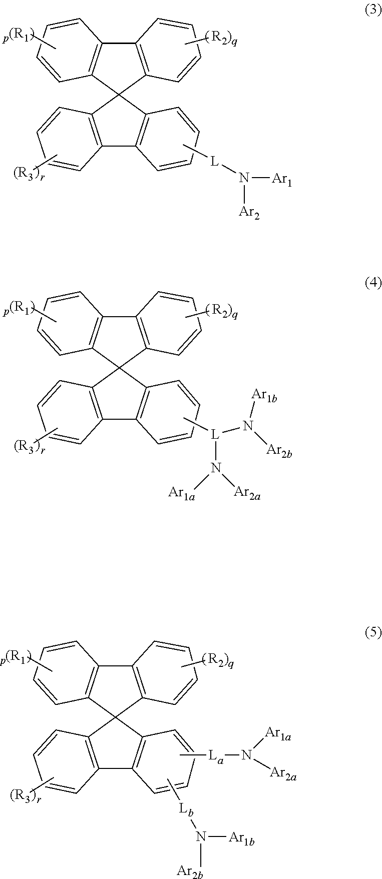

wherein Formulae 3 to 5,

Lₐ and L_{b} are as defined in L;

Ar₁ₐ and Ar_{1b} are as defined in Ar₁;

Ar₂ₐ and Ar_{2b} are as defined in Ar₂; and

L, Ar₁, Ar₂, R₁ to R₃, p, q, and r are as defined in Formula 1.

In Formulae 3 to 5, Ar₁, Ar₂, Ar₁ₐ, Ar₂ₐ, Ar_{1b}, and Ar_{2b} each independently may be represented by any one of the following Formulae R-1 to R-9.

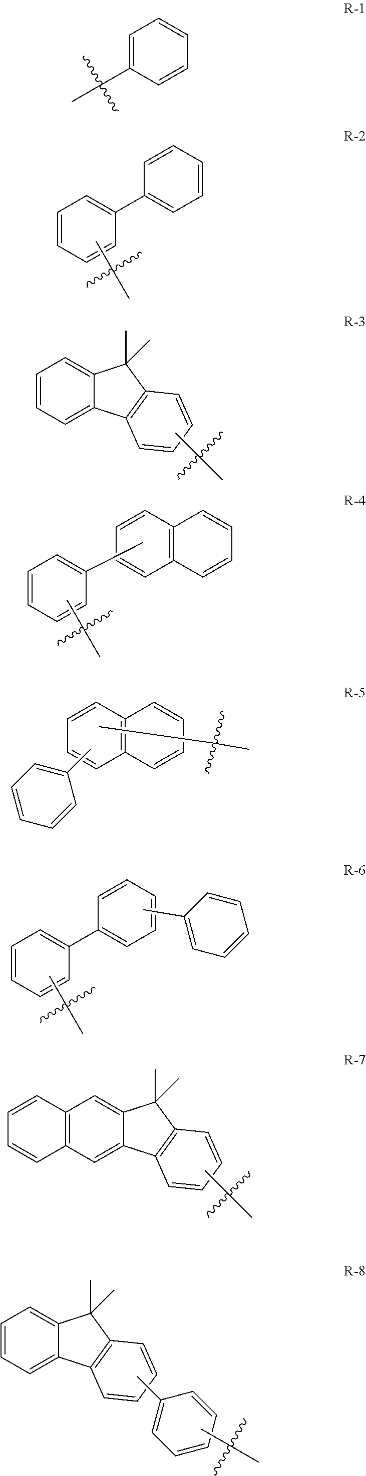

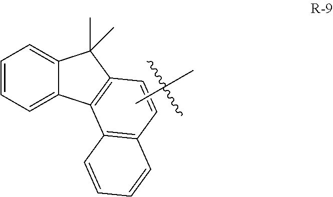

According to one embodiment of the organic electroluminescence device of the present disclosure, Formula 2 may be represented by the following Formula 6 or 7.

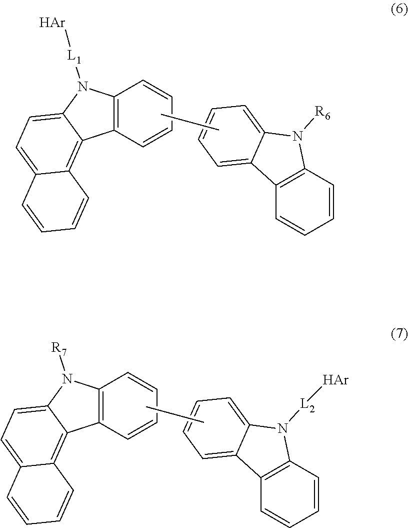

wherein Formulae 6 and 7,

HAr represents a substituted or unsubstituted (5- to 30-membered)heteroaryl containing nitrogen;

L₁ and L₂ each independently represent a single bond or a substituted or unsubstituted (C6-C30)arylene;

R₆ and R₇ each independently represent a substituted or unsubstituted (C6-C30)aryl.

In Formula 1, L represents a single bond, or a substituted or unsubstituted (C6-C30)arylene, preferably a single bond, or a substituted or unsubstituted (C6-C12)arylene, more preferably, a single bond, or unsubstituted (C6-C12)arylene. Specifically, L may represent a single bond or phenylene.

In Formula 1, Ar₁ and Ar₂ each independently represent a substituted or unsubstituted (C6-C30)aryl, or a substituted or unsubstituted (3- to 30-membered) heteroaryl, preferably a substituted or unsubstituted (C6-C20)aryl, more preferably (C1-C6)alkyl- or (C6-C12)aryl-substituted or unsubstituted (C6-C20)aryl. Specifically, Ar₁ and Ar₂ each independently may represent phenyl, biphenyl, terphenyl, naphthylphenyl, phenylnaphthyl, dimethylfluorenyl, or dimethylbenzofluorenyl.

In Formula 1, R₁ to R₃ each independently represent hydrogen, deuterium, halogen, cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; or may be linked to an adjacent substituent to form a ring of a substituted or unsubstituted, (C3-C30) mono- or polycyclic, alicyclic, aromatic, or a combination of alicyclic and aromatic ring, whose carbon atom may be replaced with at least one heteroatom selected from nitrogen, oxygen, and sulfur, preferably, hydrogen or may be linked to an adjacent substituent to form a substituted or unsubstituted, (C3-C20) mono- or polycyclic, alicyclic, aromatic, or a combination of alicyclic and aromatic ring. Specifically, R₁ to R₃ each independently represent hydrogen or may be linked to an adjacent substituent to form a benzene ring.

In Formula 2, L₁ and L₂ each independently represent a single bond, or a substituted or unsubstituted (C6-C30)arylene, preferably a single bond or a substituted or unsubstituted (C6-C12)arylene, more preferably, a single bond or an unsubstituted (C6-C12)arylene. Specifically, L₁ and L₂ each independently may represent a single bond, phenylene or naphthylene.

In Formula 2, Ar₃ and Ar₄ each independently represent a substituted or unsubstituted (C6-C30)aryl, or a substituted or unsubstituted (3- to 30-membered)heteroaryl, preferably, a substituted or unsubstituted (C6-C20)aryl, or a substituted or unsubstituted (5- to 30-membered)heteroaryl containing nitrogen, more preferably, (C1-C6)alkyl- or (C6-C12)aryl-substituted or unsubstituted (C6-C20)aryl; (C6-C20)aryl, (C1-C6)alkyl(C6-C20)aryl, or (5- to 15-membered)heteroaryl containing nitrogen substituted with (C6-C12)aryl-substituted or unsubstituted (5- to 20-membered)heteroaryl. Specifically, Ar₃ and Ar₄ each independently may represent phenyl, biphenyl, naphthylphenyl, phenylnaphthyl, terphenyl, anthracenyl, phenanthrenyl, di(C1-C6)alkylfluorenyl, quinazolinyl substituted with phenyl, quinazolinyl substituted with di(C1-C6)alkylphenyl, quinazolinyl substituted with naphthylphenyl, quinazolinyl substituted with phenylnaphthyl, quinazolinyl substituted with terphenyl, quinazolinyl substituted with anthracenyl, quinazolinyl substituted with phenanthrenyl, quinazolinyl substituted with biphenyl, quinazolinyl substituted with di(C1-C6)alkylfluorenyl, quinazolinyl substituted with phenylcarbazolyl, quinoxalinyl substituted with phenyl, quinoxalinyl substituted with naphthylphenyl, quinoxalinyl substituted with phenylnaphthyl, quinoxalinyl substituted with terphenyl, quinoxalinyl substituted with anthracenyl, quinoxalinyl substituted with phenanthrenyl, quinoxalinyl substituted with biphenyl, quinoxalinyl substituted with di(C1-C6)alkylfluorenyl, or quinoxalinyl substituted with phenylcarbazolyl.

In Formula 2, R₄ and R₅ each independently represent hydrogen, deuterium, halogen, cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; or may be linked to an adjacent substituent to form a ring of a substituted or unsubstituted, (C3-C30) mono- or polycyclic, alicyclic or aromatic, or the combination thereof, whose carbon atom may be replaced with at least one heteroatom selected from nitrogen, oxygen, and sulfur; preferably, each independently represent hydrogen or a substituted or unsubstituted (C6-C12)aryl, more preferably, each independently represent hydrogen or an unsubstituted (C6-C12)aryl. Specifically, R₄ and R₅ each independently may represent hydrogen or phenyl.

The compound represented by Formula 1 may be illustrated by the following compounds, but is not limited thereto.

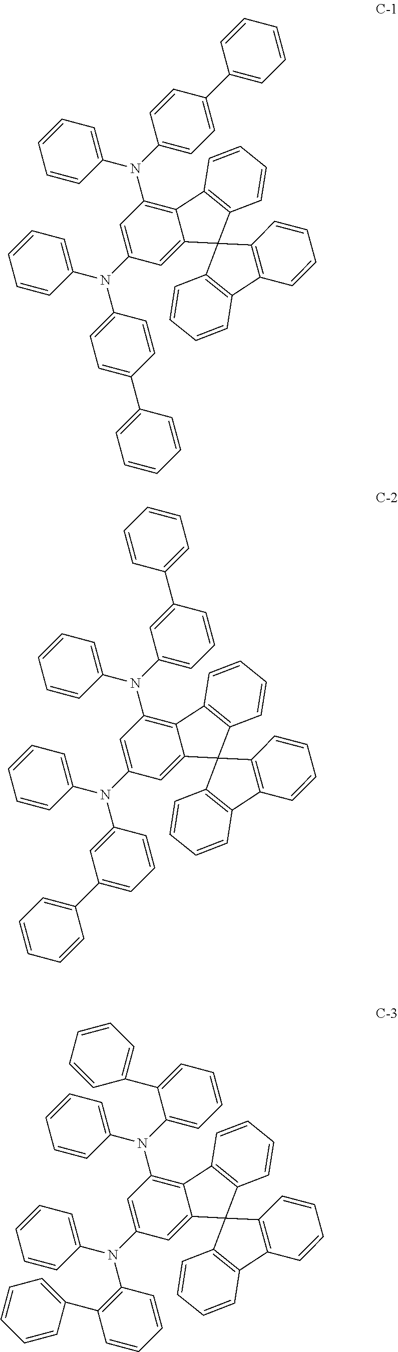

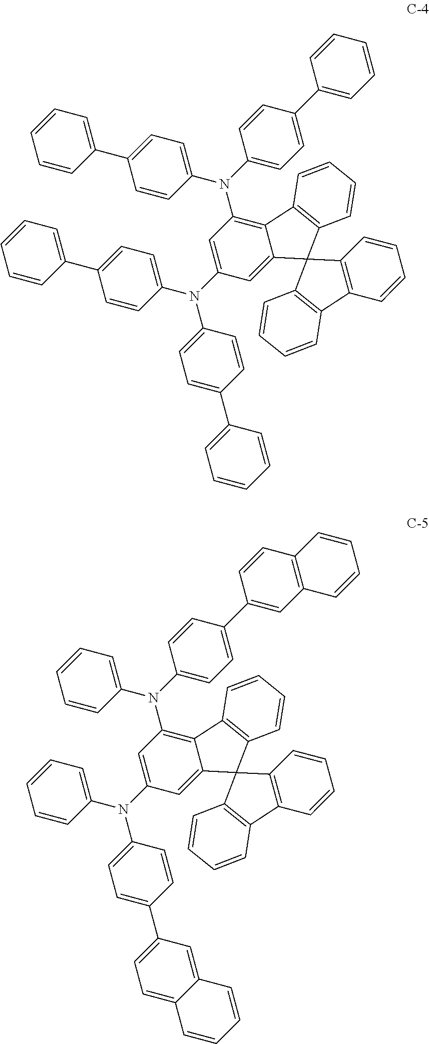 [SMILES: c1ccc(-c2ccc(N(c3ccc(-c4ccccc4)cc3)c3cc(N(c4ccc(-c5ccccc5)cc4)c4ccc(-c5ccccc5)cc4)c4c(c3)C3(c5ccccc5-c5ccccc53)c3ccccc3-4)cc2)cc1 ; c1ccc(N(c2ccc(-c3ccc4ccccc4c3)cc2)c2cc(N(c3ccccc3)c3ccc(-c4ccc5ccccc5c4)cc3)c3c(c2)C2(c4ccccc4-c4ccccc42)c2ccccc2-3)cc1]

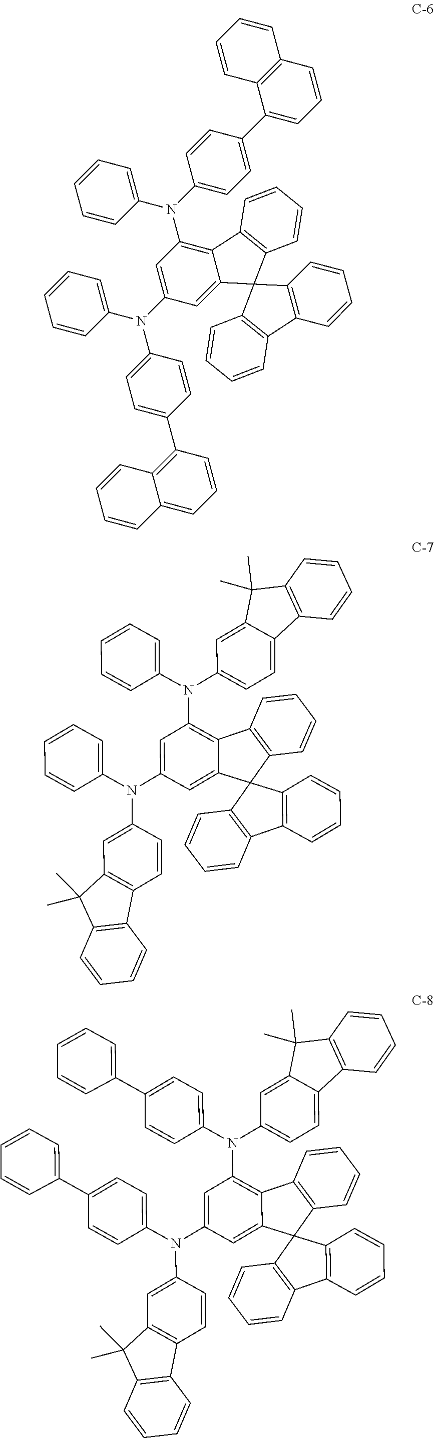

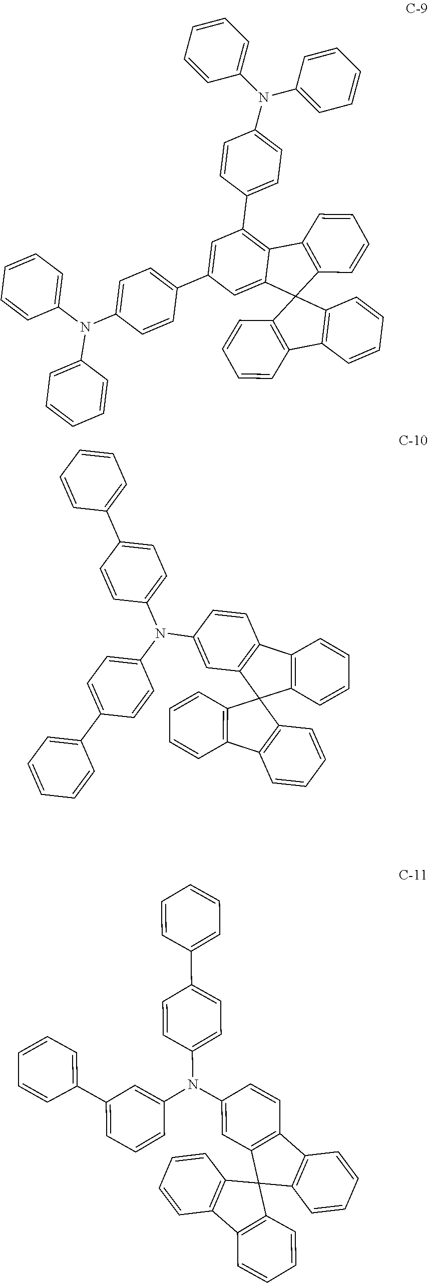

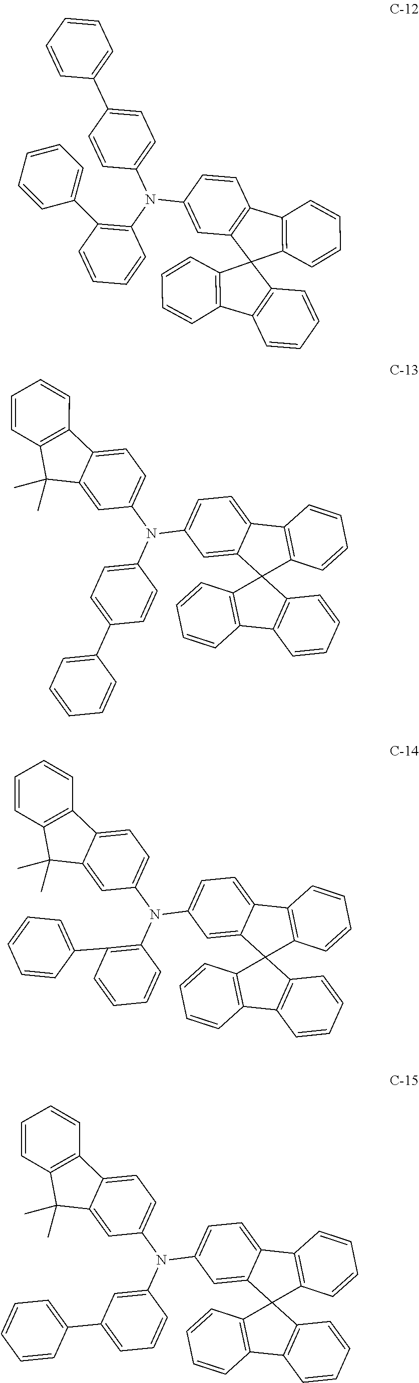 [SMILES: c1ccc(-c2ccc(N(c3ccc4c(c3)C3(c5ccccc5-c5ccccc53)c3ccccc3-4)c3ccccc3-c3ccccc3)cc2)cc1 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; CC1(C)c2ccccc2-c2ccc(N(c3cccc(-c4ccccc4)c3)c3ccc4c(c3)C3(c5ccccc5-c5ccccc53)c3ccccc3-4)cc21]

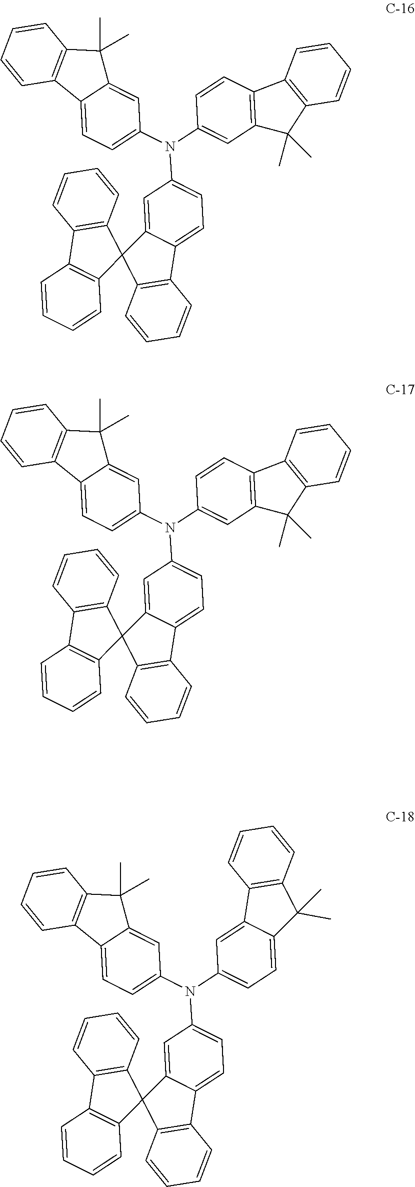 [SMILES: CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21 ; CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21 ; CC1(C)c2ccccc2-c2cc(N(c3ccc4c(c3)C(C)(C)c3ccccc3-4)c3ccc4c(c3)C3(c5ccccc5-c5ccccc53)c3ccccc3-4)ccc21]

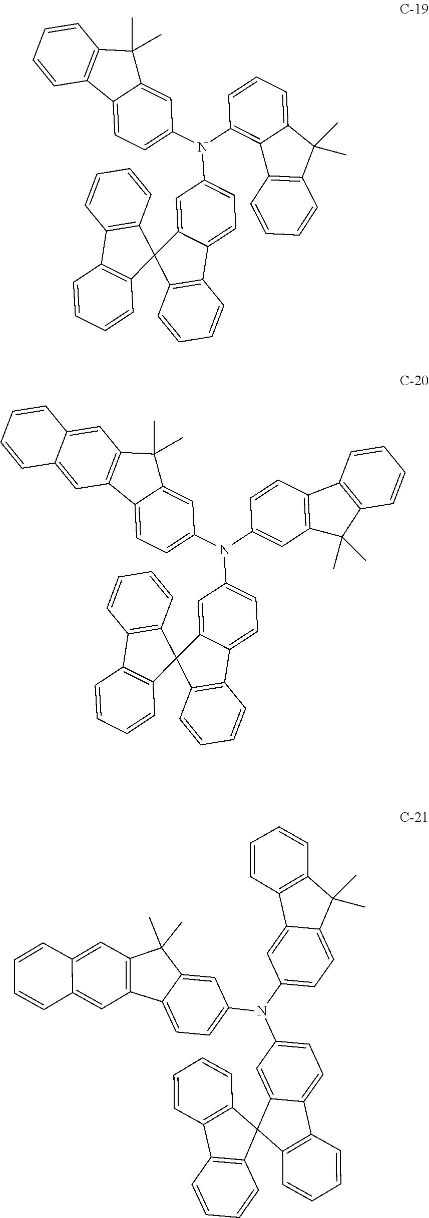 [SMILES: CC1(C)c2ccccc2-c2ccc(N(c3ccc4c(c3)C3(c5ccccc5-c5ccccc53)c3ccccc3-4)c3cccc4c3-c3ccccc3C4(C)C)cc21 ; CC1(C)c2ccccc2-c2ccc(N(c3ccc4c(c3)C(C)(C)c3cc5ccccc5cc3-4)c3ccc4c(c3)C3(c5ccccc5-c5ccccc53)c3ccccc3-4)cc21 ; CC1(C)c2ccccc2-c2cc(N(c3ccc4c(c3)C(C)(C)c3cc5ccccc5cc3-4)c3ccc4c(c3)C3(c5ccccc5-c5ccccc53)c3ccccc3-4)ccc21]

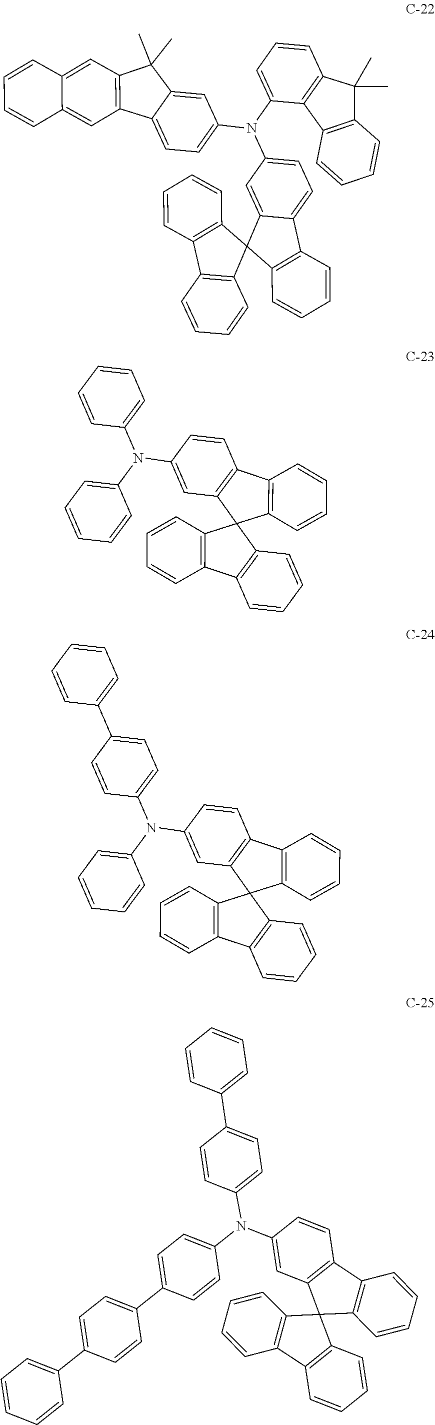 [SMILES: CC1(C)c2cc(N(c3ccc4c(c3)C3(c5ccccc5-c5ccccc53)c3ccccc3-4)c3cccc4c3-c3ccccc3C4(C)C)ccc2-c2cc3ccccc3cc21 ; c1ccc(N(c2ccccc2)c2ccc3c(c2)C2(c4ccccc4-c4ccccc42)c2ccccc2-3)cc1 ; c1ccc(-c2ccc(N(c3ccccc3)c3ccc4c(c3)C3(c5ccccc5-c5ccccc53)c3ccccc3-4)cc2)cc1 ; c1ccc(-c2ccc(-c3ccc(N(c4ccc(-c5ccccc5)cc4)c4ccc5c(c4)C4(c6ccccc6-c6ccccc64)c4ccccc4-5)cc3)cc2)cc1]

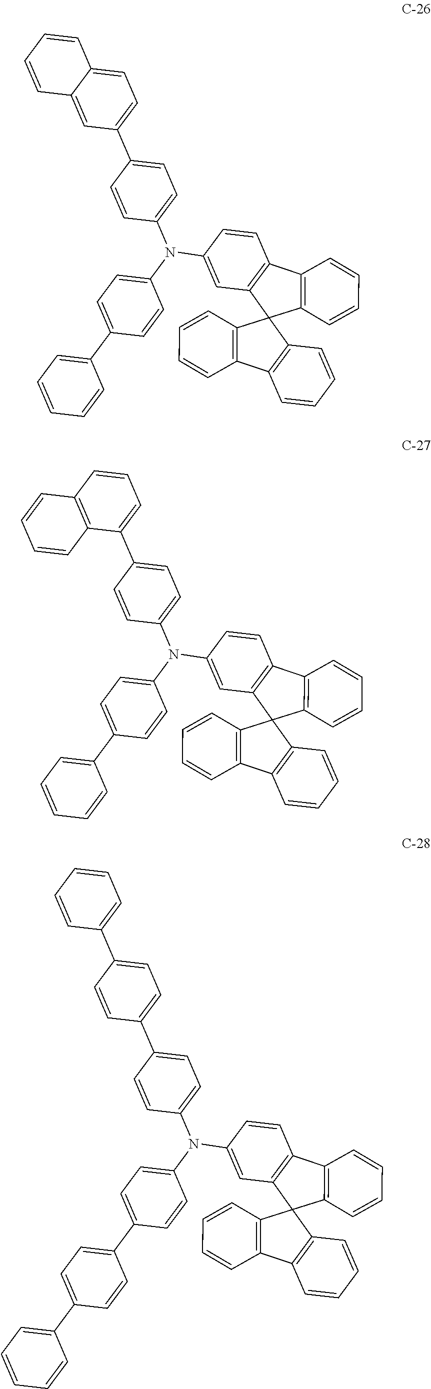 [SMILES: c1ccc(-c2ccc(N(c3ccc(-c4ccc5ccccc5c4)cc3)c3ccc4c(c3)C3(c5ccccc5-c5ccccc53)c3ccccc3-4)cc2)cc1 ; c1ccc(-c2ccc(N(c3ccc(-c4cccc5ccccc45)cc3)c3ccc4c(c3)C3(c5ccccc5-c5ccccc53)c3ccccc3-4)cc2)cc1 ; c1ccc(-c2ccc(-c3ccc(N(c4ccc(-c5ccc(-c6ccccc6)cc5)cc4)c4ccc5c(c4)C4(c6ccccc6-c6ccccc64)c4ccccc4-5)cc3)cc2)cc1]

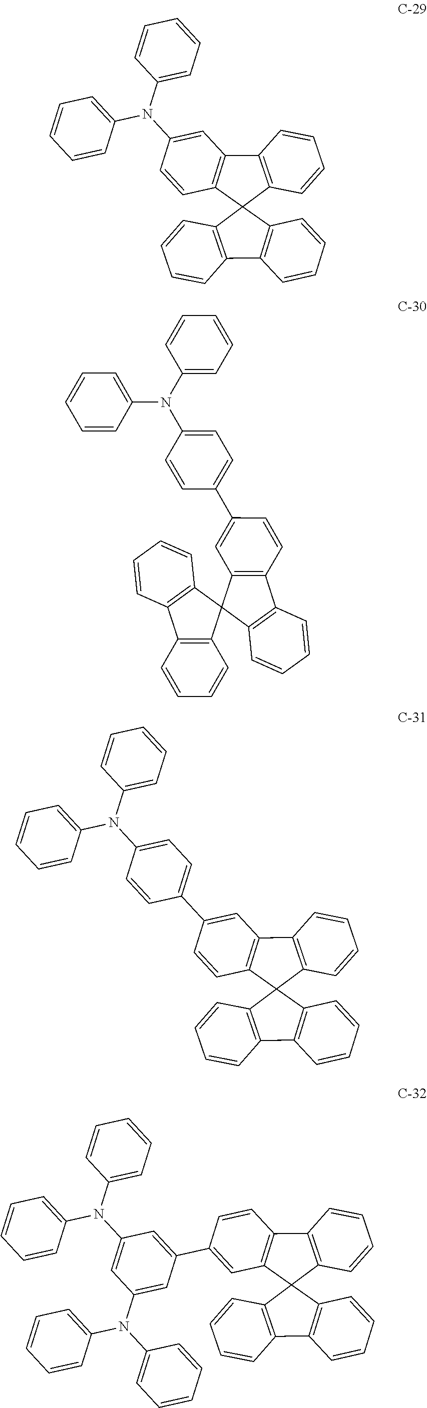

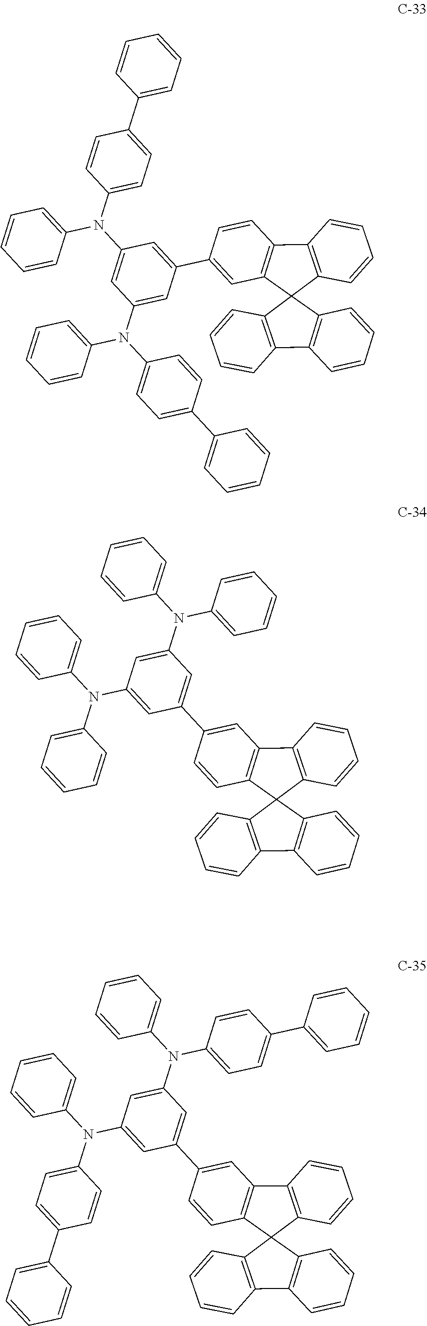 [SMILES: c1ccc(-c2ccc(N(c3ccccc3)c3cc(-c4ccc5c(c4)C4(c6ccccc6-c6ccccc64)c4ccccc4-5)cc(N(c4ccccc4)c4ccc(-c5ccccc5)cc4)c3)cc2)cc1 ; c1ccc(N(c2ccccc2)c2cc(-c3ccc4c(c3)-c3ccccc3C43c4ccccc4-c4ccccc43)cc(N(c3ccccc3)c3ccccc3)c2)cc1 ; c1ccc(-c2ccc(N(c3ccccc3)c3cc(-c4ccc5c(c4)-c4ccccc4C54c5ccccc5-c5ccccc54)cc(N(c4ccccc4)c4ccc(-c5ccccc5)cc4)c3)cc2)cc1]

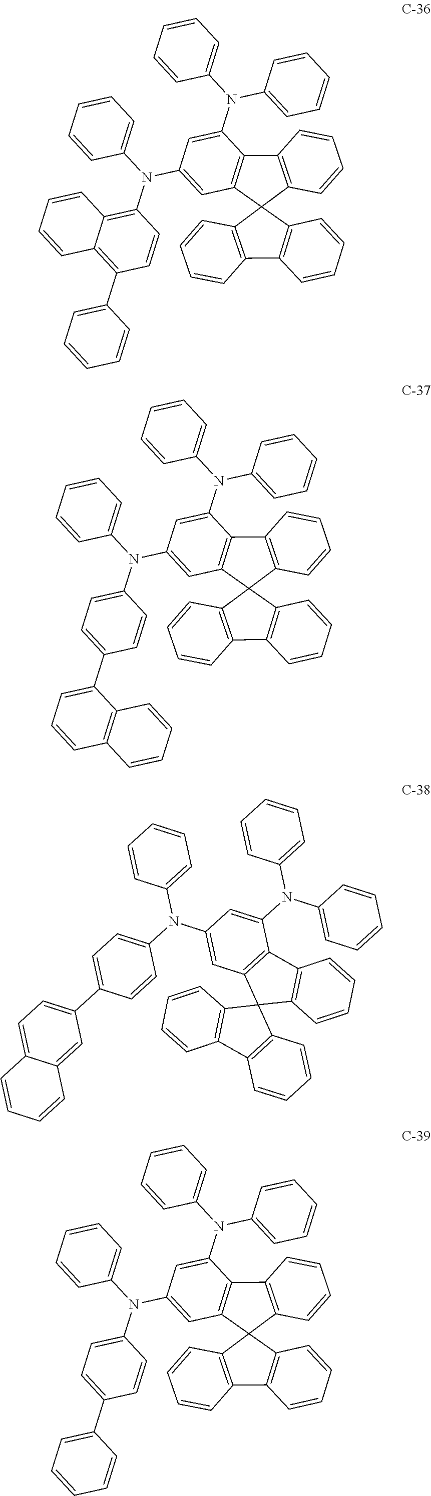 [SMILES: c1ccc(-c2ccc(N(c3ccccc3)c3cc(N(c4ccccc4)c4ccccc4)c4c(c3)C3(c5ccccc5-c5ccccc53)c3ccccc3-4)c3ccccc23)cc1 ; c1ccc(N(c2ccc(-c3cccc4ccccc34)cc2)c2cc(N(c3ccccc3)c3ccccc3)c3c(c2)C2(c4ccccc4-c4ccccc42)c2ccccc2-3)cc1 ; c1ccc(N(c2ccc(-c3ccc4ccccc4c3)cc2)c2cc(N(c3ccccc3)c3ccccc3)c3c(c2)C2(c4ccccc4-c4ccccc42)c2ccccc2-3)cc1 ; c1ccc(-c2ccc(N(c3ccccc3)c3cc(N(c4ccccc4)c4ccccc4)c4c(c3)C3(c5ccccc5-c5ccccc53)c3ccccc3-4)cc2)cc1]

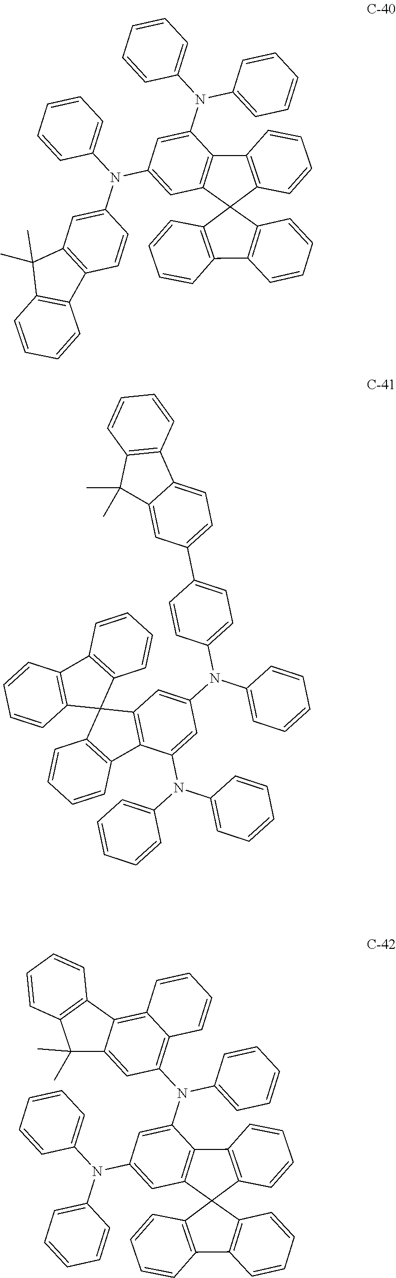 [SMILES: CC1(C)c2ccccc2-c2ccc(N(c3ccccc3)c3cc(N(c4ccccc4)c4ccccc4)c4c(c3)C3(c5ccccc5-c5ccccc53)c3ccccc3-4)cc21 ; CC1(C)c2ccccc2-c2ccc(-c3ccc(N(c4ccccc4)c4cc(N(c5ccccc5)c5ccccc5)c5c(c4)C4(c6ccccc6-c6ccccc64)c4ccccc4-5)cc3)cc21 ; CC1(C)c2ccccc2-c2c1cc(N(c1ccccc1)c1cc(N(c3ccccc3)c3ccccc3)cc3c1-c1ccccc1C31c3ccccc3-c3ccccc31)c1ccccc21]

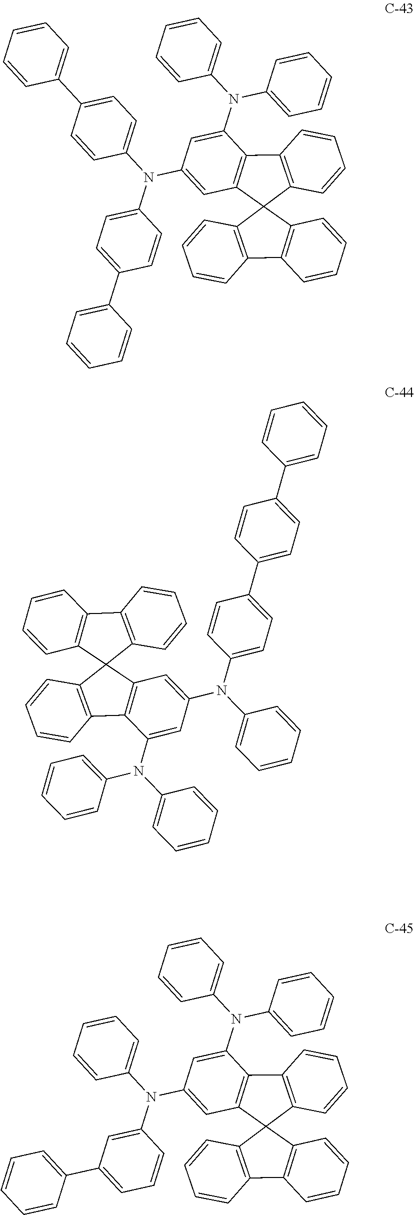 [SMILES: c1ccc(-c2ccc(N(c3ccc(-c4ccccc4)cc3)c3cc(N(c4ccccc4)c4ccccc4)c4c(c3)C3(c5ccccc5-c5ccccc53)c3ccccc3-4)cc2)cc1 ; c1ccc(-c2ccc(-c3ccc(N(c4ccccc4)c4cc(N(c5ccccc5)c5ccccc5)c5c(c4)C4(c6ccccc6-c6ccccc64)c4ccccc4-5)cc3)cc2)cc1 ; c1ccc(-c2cccc(N(c3ccccc3)c3cc(N(c4ccccc4)c4ccccc4)c4c(c3)C3(c5ccccc5-c5ccccc53)c3ccccc3-4)c2)cc1]

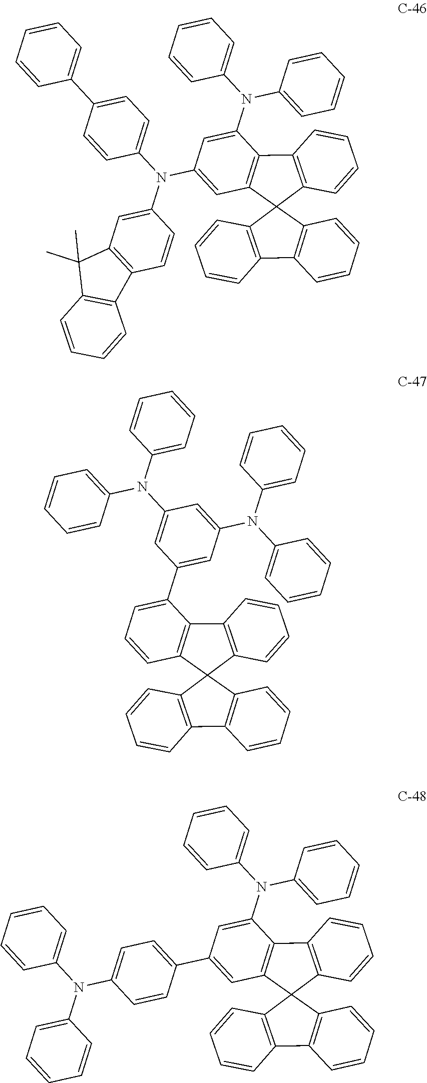 [SMILES: CC1(C)c2ccccc2-c2ccc(N(c3ccc(-c4ccccc4)cc3)c3cc(N(c4ccccc4)c4ccccc4)c4c(c3)C3(c5ccccc5-c5ccccc53)c3ccccc3-4)cc21 ; c1ccc(N(c2ccccc2)c2cc(-c3cccc4c3-c3ccccc3C43c4ccccc4-c4ccccc43)cc(N(c3ccccc3)c3ccccc3)c2)cc1 ; c1ccc(N(c2ccccc2)c2ccc(-c3cc(N(c4ccccc4)c4ccccc4)c4c(c3)C3(c5ccccc5-c5ccccc53)c3ccccc3-4)cc2)cc1]

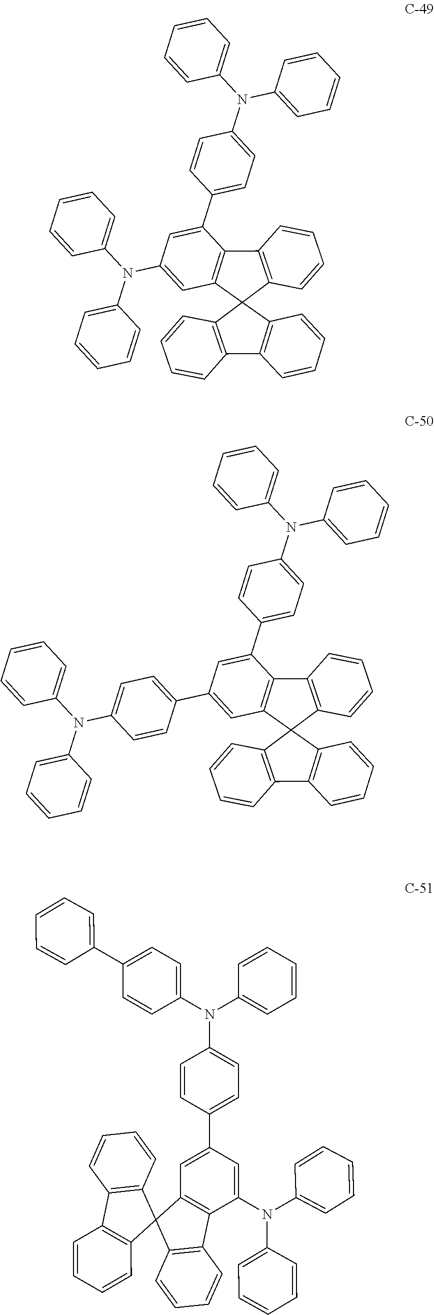 [SMILES: c1ccc(N(c2ccccc2)c2ccc(-c3cc(N(c4ccccc4)c4ccccc4)cc4c3-c3ccccc3C43c4ccccc4-c4ccccc43)cc2)cc1 ; c1ccc(N(c2ccccc2)c2ccc(-c3cc(-c4ccc(N(c5ccccc5)c5ccccc5)cc4)c4c(c3)C3(c5ccccc5-c5ccccc53)c3ccccc3-4)cc2)cc1 ; c1ccc(-c2ccc(N(c3ccccc3)c3ccc(-c4cc(N(c5ccccc5)c5ccccc5)c5c(c4)C4(c6ccccc6-c6ccccc64)c4ccccc4-5)cc3)cc2)cc1]

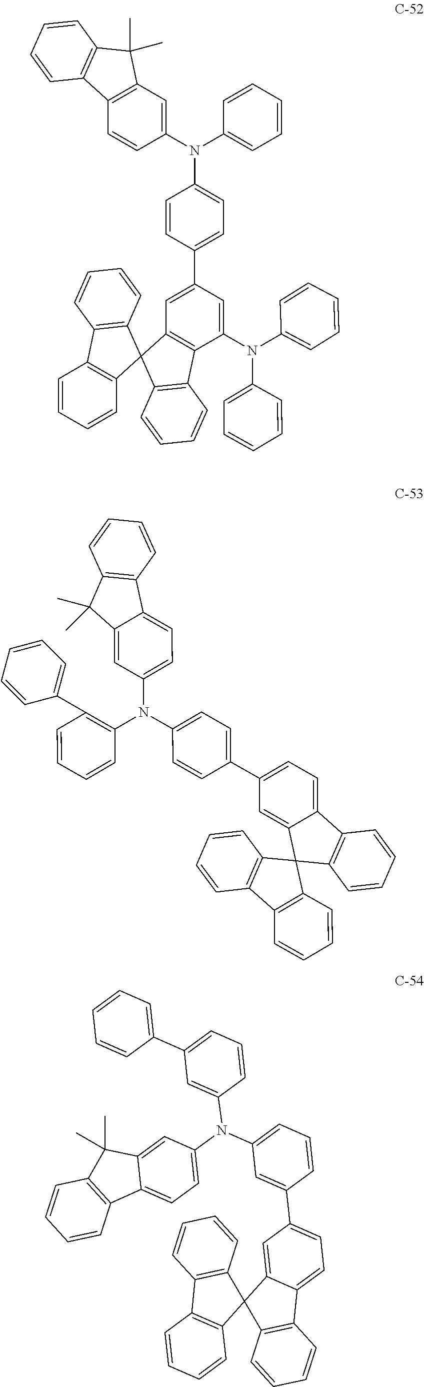 [SMILES: CC1(C)c2ccccc2-c2ccc(N(c3ccccc3)c3ccc(-c4cc(N(c5ccccc5)c5ccccc5)c5c(c4)C4(c6ccccc6-c6ccccc64)c4ccccc4-5)cc3)cc21 ; CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21 ; CC1(C)c2ccccc2-c2ccc(N(c3cccc(-c4ccccc4)c3)c3cccc(-c4ccc5c(c4)C4(c6ccccc6-c6ccccc64)c4ccccc4-5)c3)cc21]

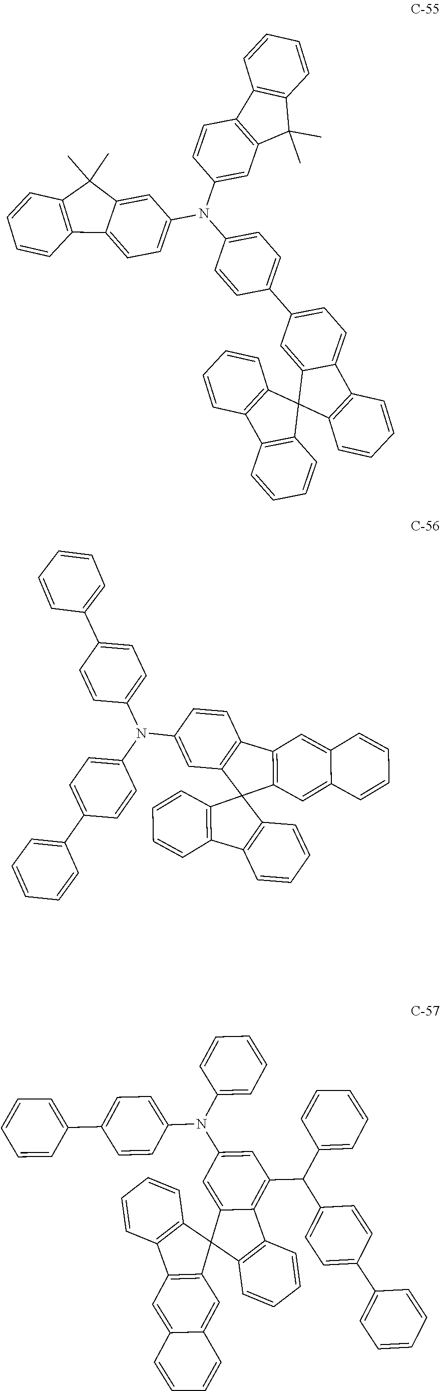 [SMILES: CC1(C)c2ccccc2-c2ccc(N(c3ccc(-c4ccc5c(c4)C4(c6ccccc6-c6ccccc64)c4ccccc4-5)cc3)c3ccc4c(c3)C(C)(C)c3ccccc3-4)cc21 ; c1ccc(-c2ccc(N(c3ccc(-c4ccccc4)cc3)c3ccc4c(c3)C3(c5ccccc5-c5ccccc53)c3cc5ccccc5cc3-4)cc2)cc1 ; c1ccc(-c2ccc(C(c3ccccc3)c3cc(N(c4ccccc4)c4ccc(-c5ccccc5)cc4)cc4c3-c3ccccc3C43c4ccccc4-c4cc5ccccc5cc43)cc2)cc1]

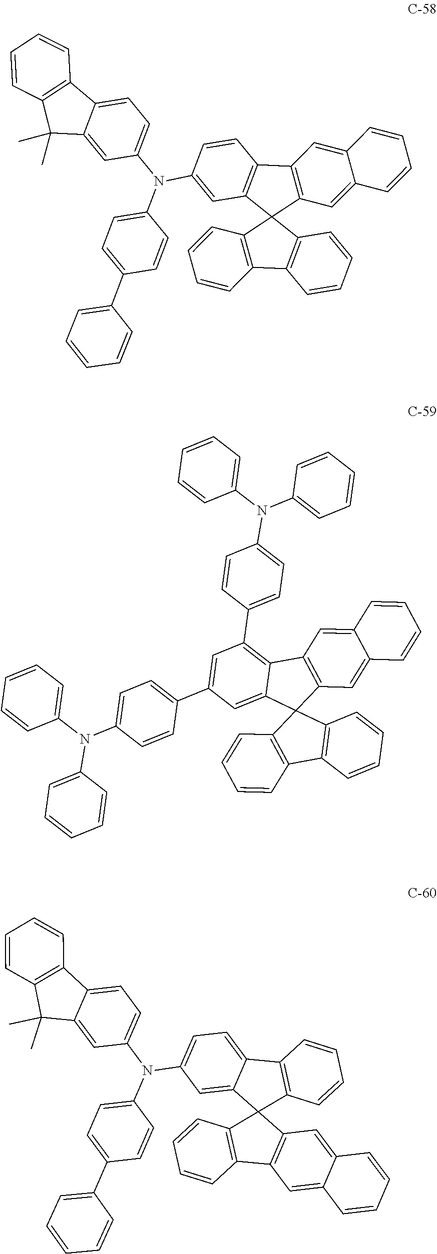 [SMILES: CC1(C)c2ccccc2-c2ccc(N(c3ccc(-c4ccccc4)cc3)c3ccc4c(c3)C3(c5ccccc5-c5ccccc53)c3cc5ccccc5cc3-4)cc21 ; c1ccc(N(c2ccccc2)c2ccc(-c3cc(-c4ccc(N(c5ccccc5)c5ccccc5)cc4)c4c(c3)C3(c5ccccc5-c5ccccc53)c3cc5ccccc5cc3-4)cc2)cc1 ; CC1(C)c2ccccc2-c2ccc(N(c3ccc(-c4ccccc4)cc3)c3ccc4c(c3)C3(c5ccccc5-4)c4ccccc4-c4cc5ccccc5cc43)cc21]

The compound represented by Formula 2 may be illustrated by the following compounds, but is not limited thereto.

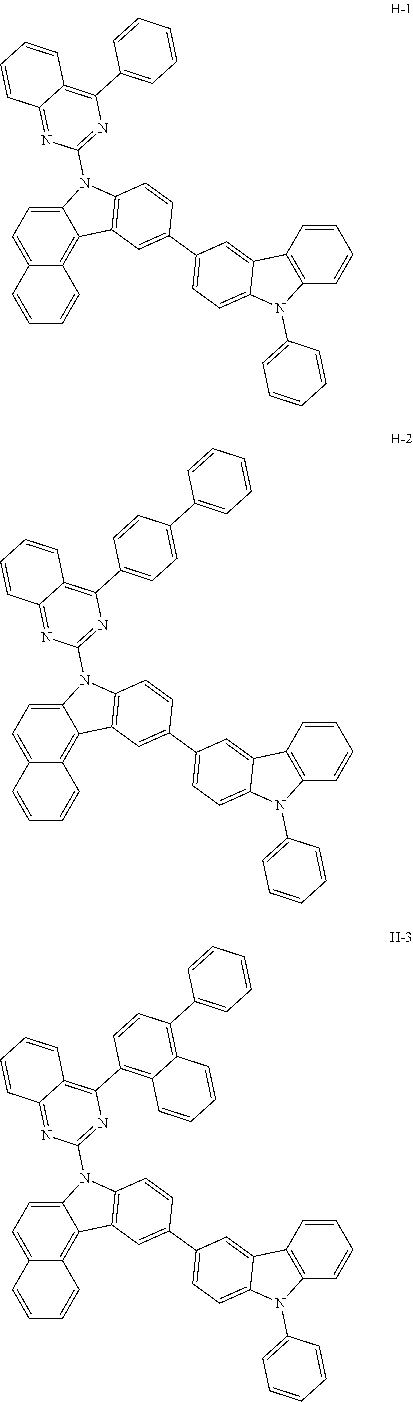 [SMILES: c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-c3nc(-n4c5ccc(-c6ccc7c(c6)c6ccccc6n7-c6ccccc6)cc5c5c6ccccc6ccc54)nc4ccccc34)cc2)cc1 ; c1ccc(-c2ccc(-c3nc(-n4c5ccc(-c6ccc7c(c6)c6ccccc6n7-c6ccccc6)cc5c5c6ccccc6ccc54)nc4ccccc34)c3ccccc23)cc1]

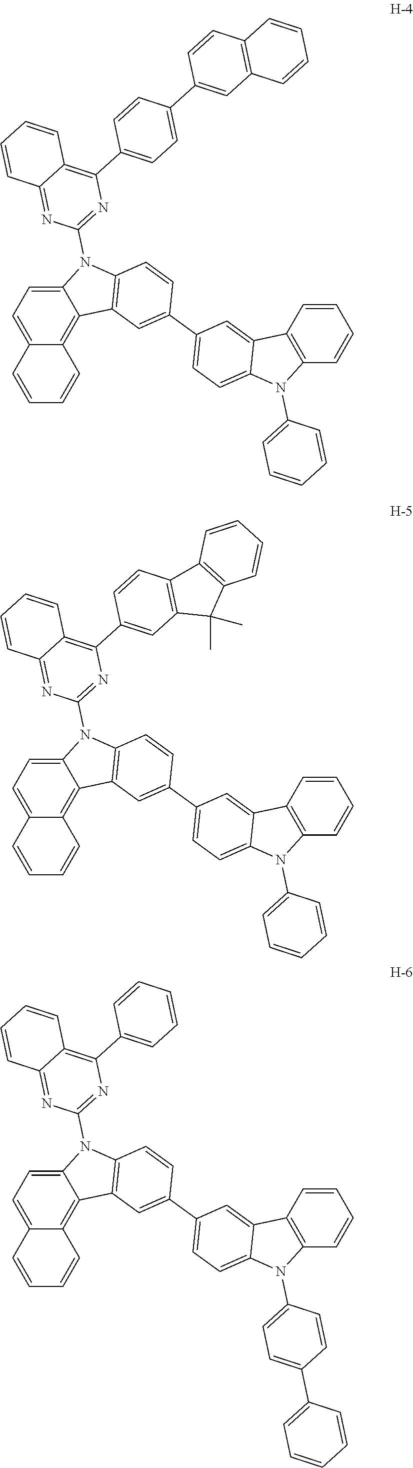 [SMILES: c1ccc(-n2c3ccccc3c3cc(-c4ccc5c(c4)c4c6ccccc6ccc4n5-c4nc(-c5ccc(-c6ccc7ccccc7c6)cc5)c5ccccc5n4)ccc32)cc1 ; CC1(C)c2ccccc2-c2ccc(-c3nc(-n4c5ccc(-c6ccc7c(c6)c6ccccc6n7-c6ccccc6)cc5c5c6ccccc6ccc54)nc4ccccc34)cc21 ; c1ccc(-c2ccc(-n3c4ccccc4c4cc(-c5ccc6c(c5)c5c7ccccc7ccc5n6-c5nc(-c6ccccc6)c6ccccc6n5)ccc43)cc2)cc1]

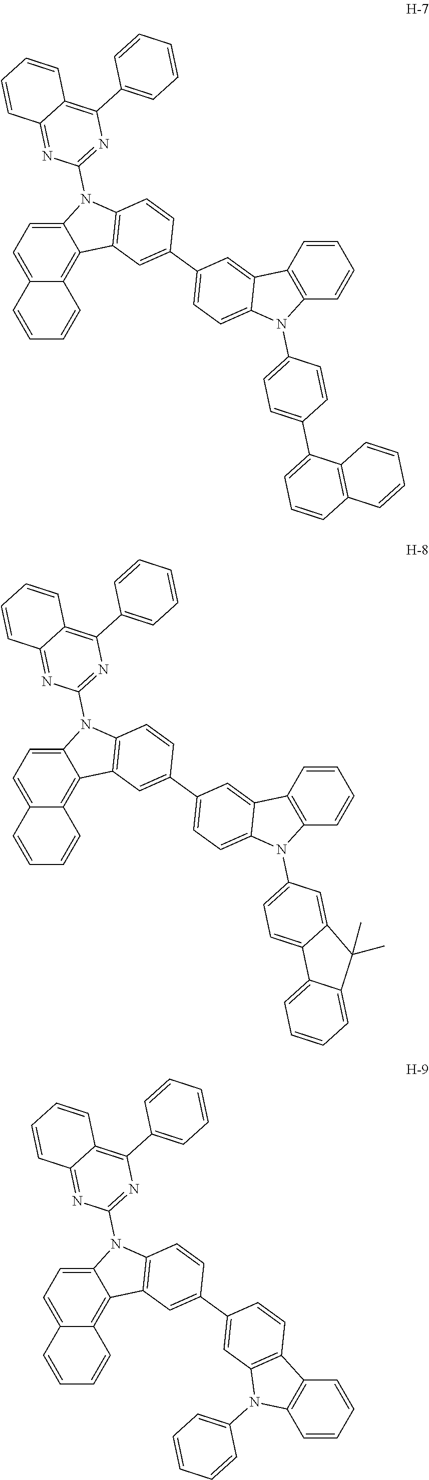 [SMILES: c1ccc(-c2nc(-n3c4ccc(-c5ccc6c(c5)c5ccccc5n6-c5ccc(-c6cccc7ccccc67)cc5)cc4c4c5ccccc5ccc43)nc3ccccc23)cc1 ; CC1(C)c2ccccc2-c2ccc(-n3c4ccccc4c4cc(-c5ccc6c(c5)c5c7ccccc7ccc5n6-c5nc(-c6ccccc6)c6ccccc6n5)ccc43)cc21 ; c1ccc(-c2nc(-n3c4ccc(-c5ccc6c7ccccc7n(-c7ccccc7)c6c5)cc4c4c5ccccc5ccc43)nc3ccccc23)cc1]

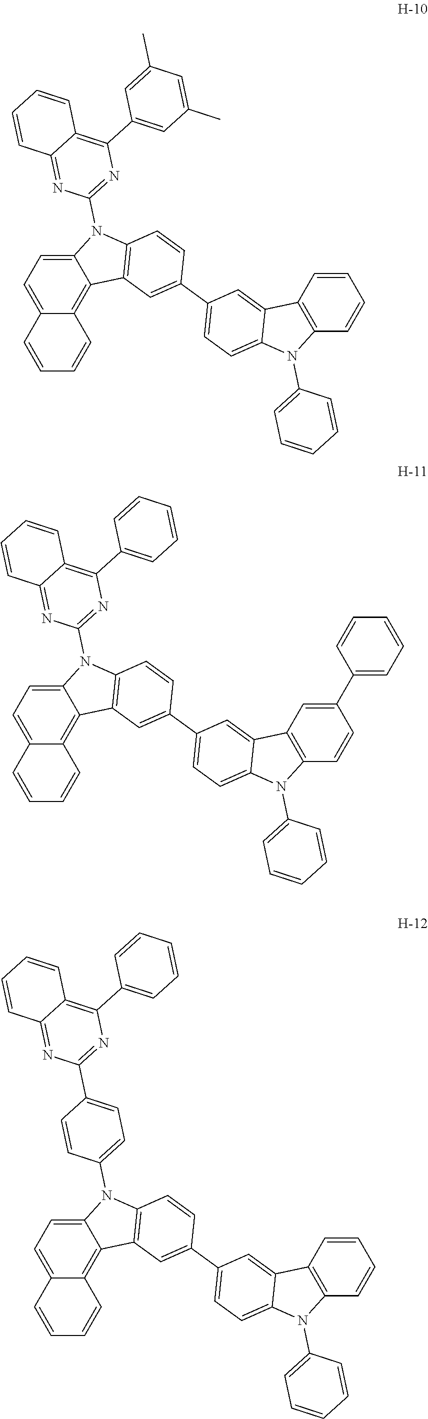 [SMILES: Cc1cc(C)cc(-c2nc(-n3c4ccc(-c5ccc6c(c5)c5ccccc5n6-c5ccccc5)cc4c4c5ccccc5ccc43)nc3ccccc23)c1 ; c1ccc(-c2ccc3c(c2)c2cc(-c4ccc5c(c4)c4c6ccccc6ccc4n5-c4nc(-c5ccccc5)c5ccccc5n4)ccc2n3-c2ccccc2)cc1 ; c1ccc(-c2nc(-c3ccc(-n4c5ccc(-c6ccc7c(c6)c6ccccc6n7-c6ccccc6)cc5c5c6ccccc6ccc54)cc3)nc3ccccc23)cc1]

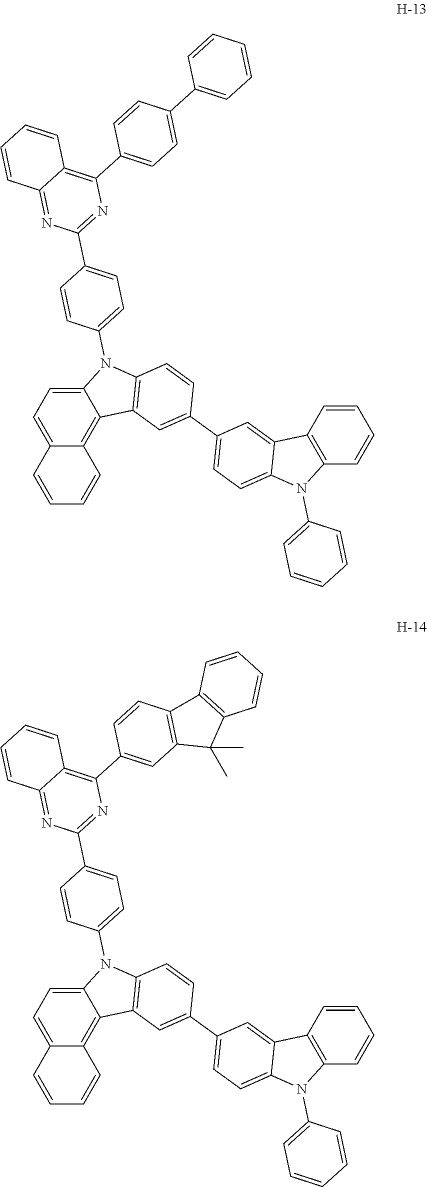 [SMILES: c1ccc(-c2ccc(-c3nc(-c4ccc(-n5c6ccc(-c7ccc8c(c7)c7ccccc7n8-c7ccccc7)cc6c6c7ccccc7ccc65)cc4)nc4ccccc34)cc2)cc1 ; CC1(C)c2ccccc2-c2ccc(-c3nc(-c4ccc(-n5c6ccc(-c7ccc8c(c7)c7ccccc7n8-c7ccccc7)cc6c6c7ccccc7ccc65)cc4)nc4ccccc34)cc21]

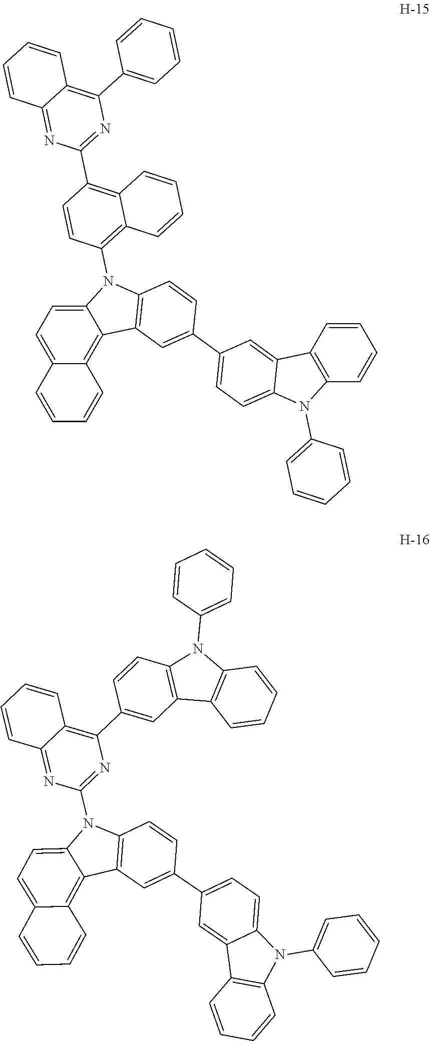 [SMILES: c1ccc(-c2nc(-c3ccc(-n4c5ccc(-c6ccc7c(c6)c6ccccc6n7-c6ccccc6)cc5c5c6ccccc6ccc54)c4ccccc34)nc3ccccc23)cc1 ; c1ccc(-n2c3ccccc3c3cc(-c4ccc5c(c4)c4c6ccccc6ccc4n5-c4nc(-c5ccc6c(c5)c5ccccc5n6-c5ccccc5)c5ccccc5n4)ccc32)cc1]

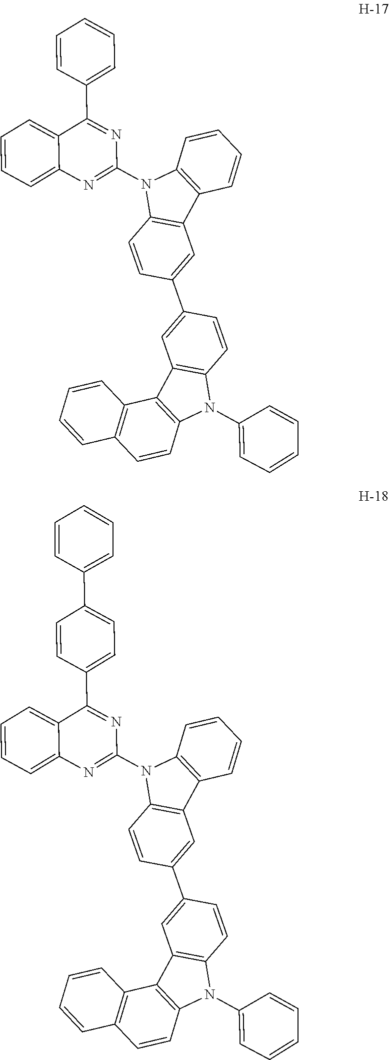 [SMILES: c1ccc(-c2nc(-n3c4ccccc4c4cc(-c5ccc6c(c5)c5c7ccccc7ccc5n6-c5ccccc5)ccc43)nc3ccccc23)cc1 ; c1ccc(-c2ccc(-c3nc(-n4c5ccccc5c5cc(-c6ccc7c(c6)c6c8ccccc8ccc6n7-c6ccccc6)ccc54)nc4ccccc34)cc2)cc1]

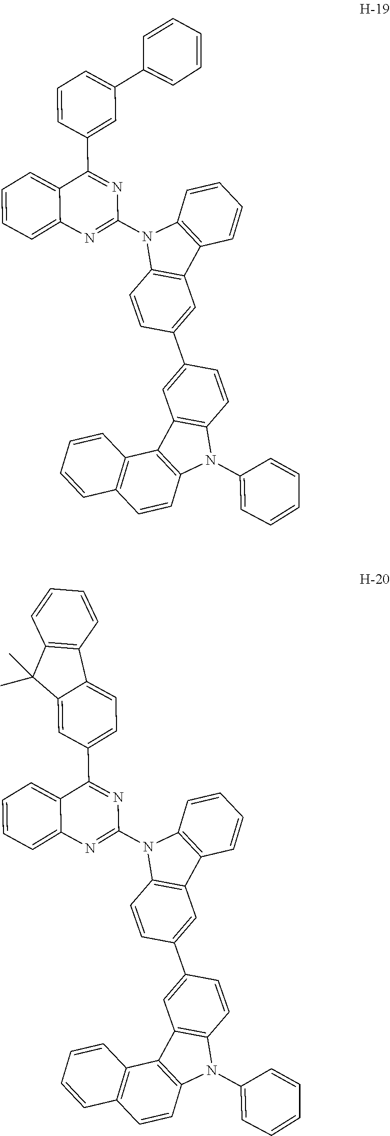 [SMILES: c1ccc(-c2cccc(-c3nc(-n4c5ccccc5c5cc(-c6ccc7c(c6)c6c8ccccc8ccc6n7-c6ccccc6)ccc54)nc4ccccc34)c2)cc1 ; CC1(C)c2ccccc2-c2ccc(-c3nc(-n4c5ccccc5c5cc(-c6ccc7c(c6)c6c8ccccc8ccc6n7-c6ccccc6)ccc54)nc4ccccc34)cc21]

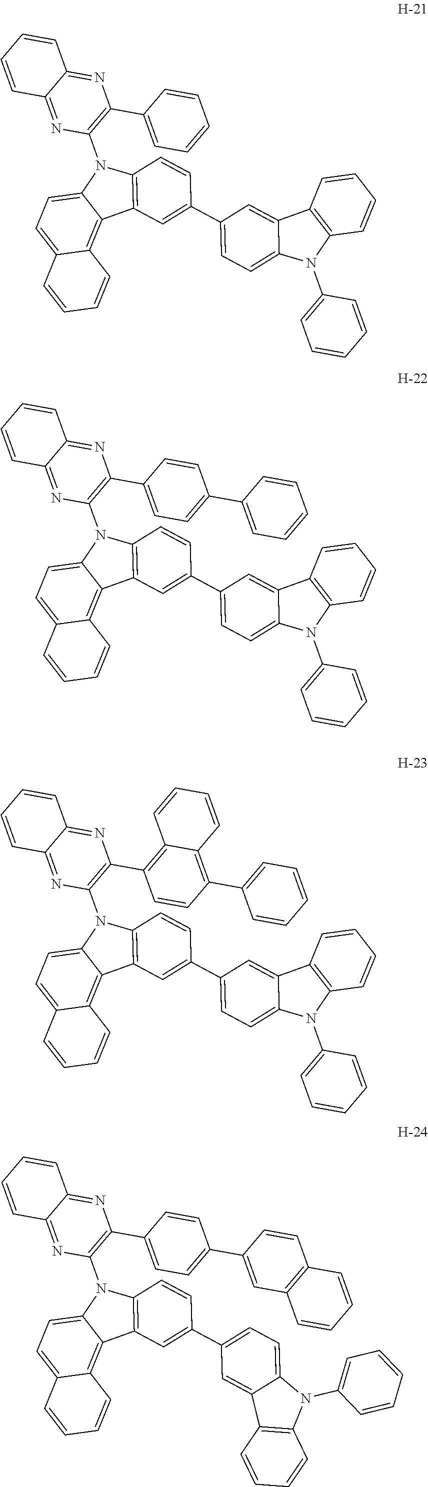 [SMILES: c1ccc(-c2nc3ccccc3nc2-n2c3ccc(-c4ccc5c(c4)c4ccccc4n5-c4ccccc4)cc3c3c4ccccc4ccc32)cc1 ; c1ccc(-c2ccc(-c3nc4ccccc4nc3-n3c4ccc(-c5ccc6c(c5)c5ccccc5n6-c5ccccc5)cc4c4c5ccccc5ccc43)cc2)cc1 ; c1ccc(-c2ccc(-c3nc4ccccc4nc3-n3c4ccc(-c5ccc6c(c5)c5ccccc5n6-c5ccccc5)cc4c4c5ccccc5ccc43)c3ccccc23)cc1 ; c1ccc(-n2c3ccccc3c3cc(-c4ccc5c(c4)c4c6ccccc6ccc4n5-c4nc5ccccc5nc4-c4ccc(-c5ccc6ccccc6c5)cc4)ccc32)cc1]

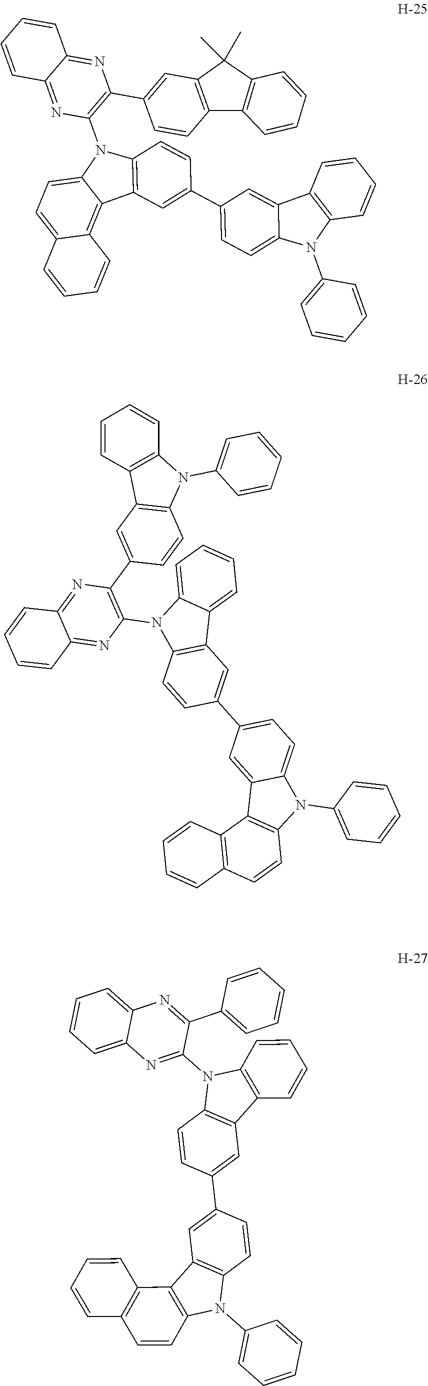 [SMILES: CC1(C)c2ccccc2-c2ccc(-c3nc4ccccc4nc3-n3c4ccc(-c5ccc6c(c5)c5ccccc5n6-c5ccccc5)cc4c4c5ccccc5ccc43)cc21 ; c1ccc(-n2c3ccccc3c3cc(-c4nc5ccccc5nc4-n4c5ccccc5c5cc(-c6ccc7c(c6)c6c8ccccc8ccc6n7-c6ccccc6)ccc54)ccc32)cc1 ; c1ccc(-c2nc3ccccc3nc2-n2c3ccccc3c3cc(-c4ccc5c(c4)c4c6ccccc6ccc4n5-c4ccccc4)ccc32)cc1]

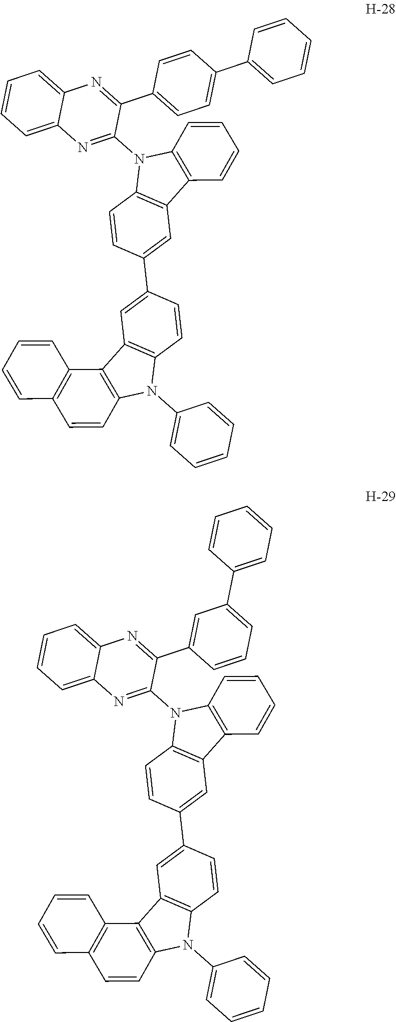 [SMILES: c1ccc(-c2ccc(-c3nc4ccccc4nc3-n3c4ccccc4c4cc(-c5ccc6c(c5)c5c7ccccc7ccc5n6-c5ccccc5)ccc43)cc2)cc1 ; c1ccc(-c2cccc(-c3nc4ccccc4nc3-n3c4ccccc4c4cc(-c5ccc6c(c5)c5c7ccccc7ccc5n6-c5ccccc5)ccc43)c2)cc1]

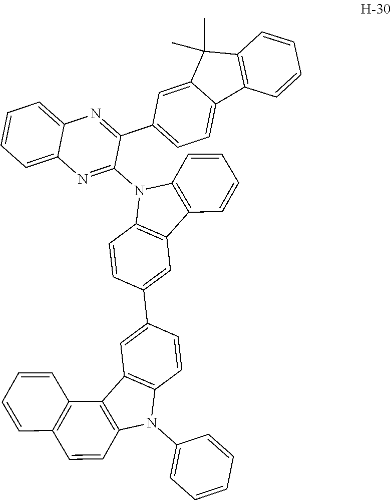 [SMILES: CC1(C)c2ccccc2-c2ccc(-c3nc4ccccc4nc3-n3c4ccccc4c4cc(-c5ccc6c(c5)c5c7ccccc7ccc5n6-c5ccccc5)ccc43)cc21]

An organic electroluminescence device according to the present disclosure comprises a first electrode; a second electrode facing the first electrode; an intermediate layer between the first electrode and the second electrode; wherein the intermediate layer comprises at least one layer of hole transporting band and at least one layer of light-emitting layer; wherein at least one layer of the hole transporting band comprises a compound represented by Formula 1; wherein at least one layer of the light-emitting layer comprises at least one dopant compound and at least one host compound; and at least one host compound comprises a compound represented by Formula 2.

In addition to the light-emitting layer and the hole transporting band, the intermediate layer may further include one or more layers selected from a light-emitting auxiliary layer, an electron transport layer, an electron buffer layer, an electron injection layer, an interlayer, and a hole blocking layer.

The hole transporting band of the present disclosure may be composed of one or more layers from the group consisting of a hole transport layer, a hole injection layer, an electron blocking layer and a hole auxiliary layer, and each of the layers may be formed of one or more layers.

Preferably, the hole transporting band includes a hole transport layer. In addition, the hole transporting band may include a hole transport layer, and may further include at least one layer of a hole injection layer, an electron blocking layer, and a hole auxiliary layer.

Herein, the hole auxiliary layer or the light-emitting auxiliary layer is disposed between the hole transport layer and the light-emitting layer and controls transport speed of the hole. The hole auxiliary layer or the light-emitting auxiliary layer provides effects of improving the efficiency and lifespan of the organic electroluminescence device.

According to one embodiment of the present disclosure, the hole transport layer may be a single layer, and may include a hole transport material comprising a compound represented by Formula 1 of the present disclosure.

According to another embodiment of the present disclosure, the hole transporting band includes a hole transport layer, and the hole transport layer may be composed of two or more layers, wherein at least one of a plurality of layers may include a hole transport material comprising a compound represented by Formula 1 of the present disclosure. The hole transport layer comprising a compound of Formula 1 or other layers may comprise all compounds used in conventional hole transport material, e.g., may comprise a compound represented by the following Formula 10:

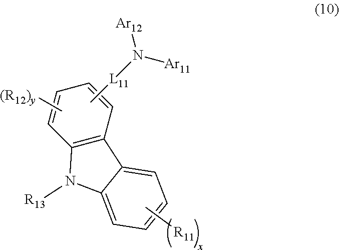

wherein Formula 10,

L₁₁ represents a single bond, a substituted or unsubstituted (C6-C30)arylene, or a substituted or unsubstituted (5- to 30-membered)heteroarylene;

Ar₁₁ and Ar₁₂ each independently represent substituted or unsubstituted (C6-C30)aryl or a substituted or unsubstituted (5- to 30-membered)heteroaryl, or Ar₁₁ and L₁₁ may form (5- to 30-membered)heteroaryl containing nitrogen together with bonded nitrogen;

R₁₁ to R₁₃ each independently represent hydrogen, deuterium, halogen, cyano, carboxyl, nitro, hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (5- to 30-membered)heteroaryl, —NR₄₁R₄₂, —SiR₄₃R₄₄R₄₅, —SR₄₆, —OR₄₇, —COR₄₈ or —B(OR₄₉)(OR₅₀); or may be linked to an adjacent substituent to form a ring of a substituted or unsubstituted, (3- to 30-membered) mono- or polycyclic, alicyclic or aromatic, or the combination thereof; whose carbon atom may be replaced with at least one heteroatom selected from nitrogen, oxygen, and sulfur;

R₄₁ to R₅₀ each independently represent hydrogen, deuterium, halogen, cyano, carboxyl, nitro, hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (5- to 30-membered)heteroaryl, or may be linked to an adjacent substituent to form a ring of a substituted or unsubstituted, (3- to 30-membered) mono- or polycyclic, alicyclic or aromatic, or the combination thereof, whose carbon atom may be replaced with at least one heteroatom selected from nitrogen, oxygen, and sulfur;

x represents an integer of 1 to 4, where if x represents an integer of 2 or more, each of R₁₁ may be the same or different;

y represents an integer of 1 to 3, where if y represents an integer of 2 or more, each of R₁₂ may be the same or different;

the heteroaryl(ene) contains at least one heteroatom selected from B, N, O, S, Si and P;

the heterocycloalkyl contains at least one heteroatom selected from O, S, and N.

The compound of the present disclosure represented by Formula 2 may be comprised in the light-emitting layer. When used in the light-emitting layer, the organic electroluminescence compound of Formula 2 may be comprised as a host material. Preferably, the light-emitting layer may further comprise at least one dopant. If necessary, the compound of the present disclosure represented by Formula 2 may be used as a co-host material. That is, the light-emitting layer may comprise the organic electroluminescence compound of Formula 2 of the present disclosure (a first host material) and may further comprise a compound other than the first host material as a second host material. Herein, the weight ratio of the first host material to the second host material is in the range of 1:99 to 99:1.

Any of the known phosphorescent hosts are available for use as the second host material. In terms of luminous efficiency, the second host material may be preferably selected from the group consisting of the compounds represented by the following Formulae 11 to 16:

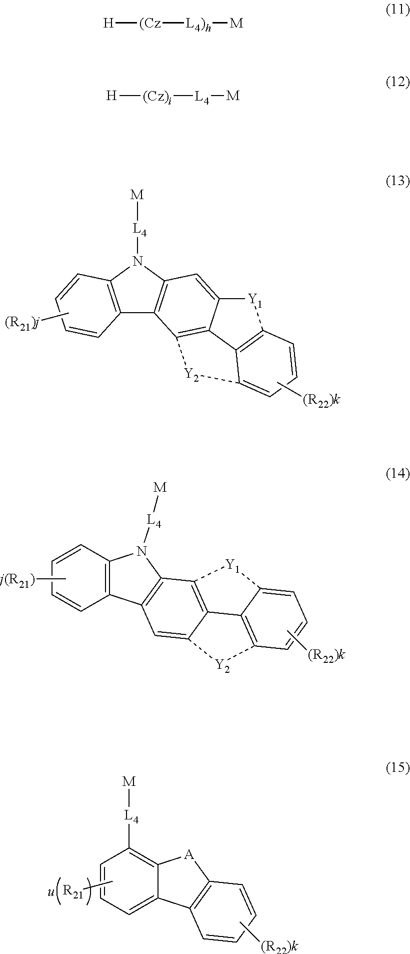

wherein Formulae 11 to 15,

Cz represents the following structure:

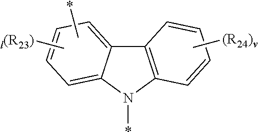

A represents —O— or —S—; and

R₂₁ to R₂₄, each independently, represent hydrogen, deuterium, halogen, a substituted or unsubstituted (C1-C30)alkyl, a substituted or unsubstituted (C6-C30)aryl, a substituted or unsubstituted (5- to 30-membered)heteroaryl, or —SiR₂₅R₂₆R₂₇, in which R₂₅ to R₂₇, each independently, represent a substituted or unsubstituted (C1-C30)alkyl or a substituted or unsubstituted (C6-C30)aryl; L₄ represents a single bond, a substituted or unsubstituted (C6-C30)arylene, or a substituted or unsubstituted (5- to 30-membered)heteroarylene; M represents a substituted or unsubstituted (C6-C30)aryl, or a substituted or unsubstituted (5- to 30-membered)heteroaryl; Y₁ and Y₂, each independently, represent —O—, —S—, —NR₃₁— or —CR₃₂R₃₃—, with the proviso that Y₁ and Y₂ are not present simultaneously; R₃₁ to R₃₃, each independently, represent a substituted or unsubstituted (C1-C30)alkyl, a substituted or unsubstituted (C6-C30)aryl, or a substituted or unsubstituted (5- to 30-membered)heteroaryl; R₃₂ and R₃₃ may be the same or different; h and i, each independently, represent an integer of 1 to 3; j, k, l, and v, each independently, represent an integer of 0 to 4; u represents an integer of 0 to 3; where if h, i, j, k, l, u or v represents an integer of 2 or more, each (Cz-L₄), each (Cz), each R₂₁, each R₂₂, each R₂₃ or each R₂₄ may be the same or different.

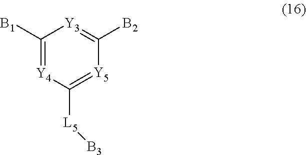

wherein Formula 16,

Y₃ to Y₅, each independently, represent CR₃₄ or N;

R₃₄ represents hydrogen, a substituted or unsubstituted (C1-C30)alkyl, a substituted or unsubstituted (C6-C30)aryl, or a substituted or unsubstituted (5- to 30-membered)heteroaryl;

B₁ and B₂, each independently, represent hydrogen, a substituted or unsubstituted (C6-C30)aryl, or a substituted or unsubstituted (5- to 30-membered)heteroaryl;

B₃ represents a substituted or unsubstituted (C6-C30)aryl, or a substituted or unsubstituted (5- to 30-membered)heteroaryl; and

L₅ represents a single bond, a substituted or unsubstituted (C6-C30)arylene, or a substituted or unsubstituted (5- to 30-membered)heteroarylene.

Specifically, the preferred examples of the second host material are as follows.

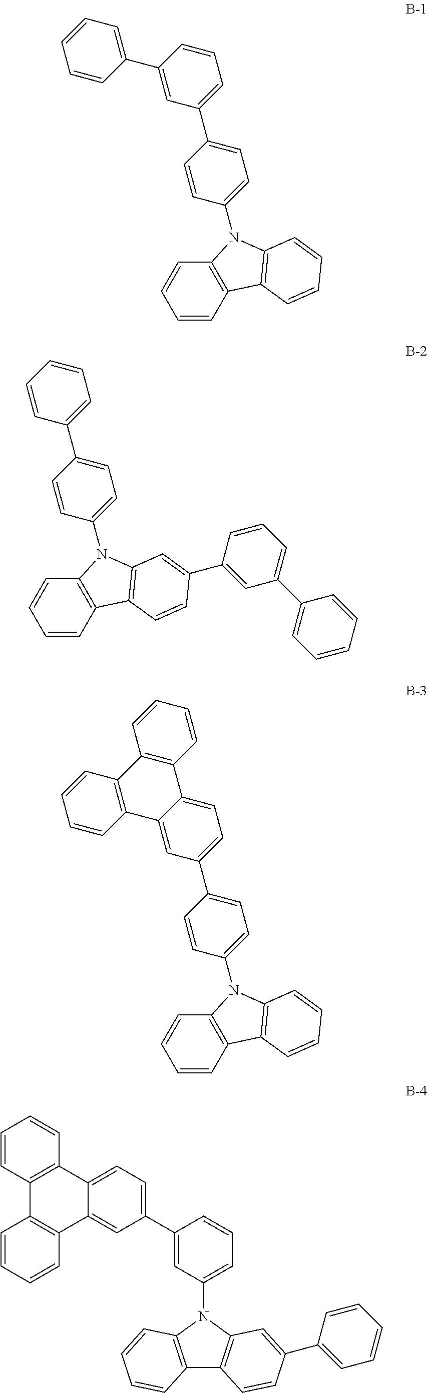 [SMILES: c1ccc(-c2cccc(-c3ccc(-n4c5ccccc5c5ccccc54)cc3)c2)cc1 ; c1ccc(-c2ccc(-n3c4ccccc4c4ccc(-c5cccc(-c6ccccc6)c5)cc43)cc2)cc1 ; c1ccc2c(c1)c1ccccc1c1cc(-c3ccc(-n4c5ccccc5c5ccccc54)cc3)ccc21 ; c1ccc(-c2ccc3c4ccccc4n(-c4cccc(-c5ccc6c7ccccc7c7ccccc7c6c5)c4)c3c2)cc1]

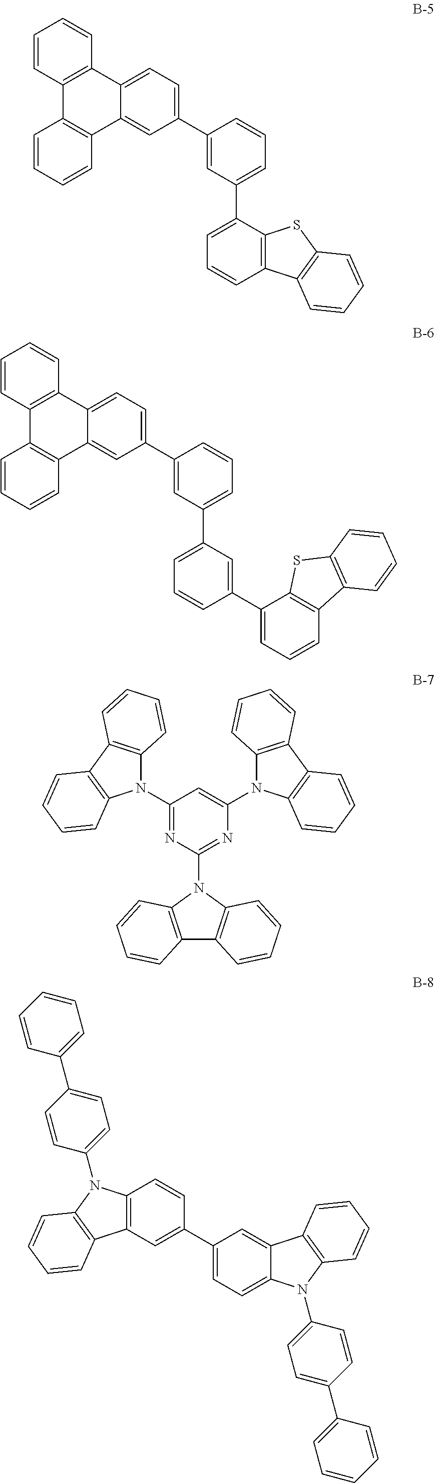

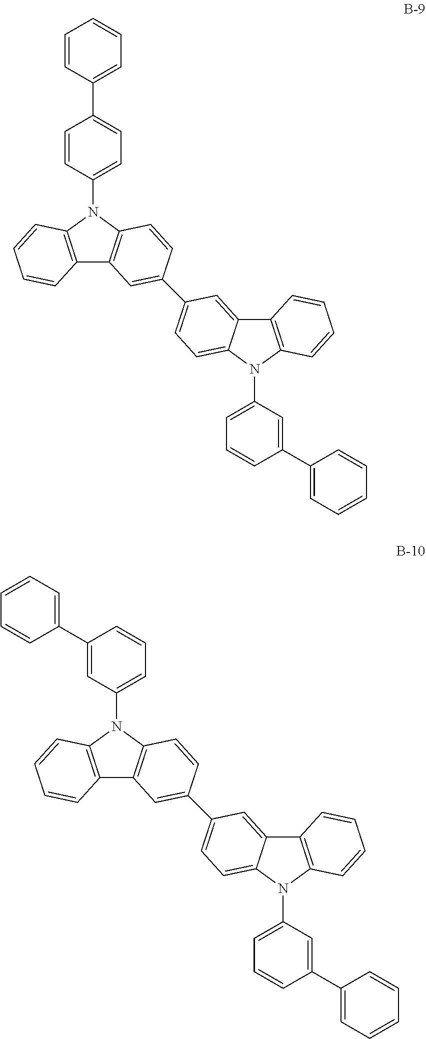 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

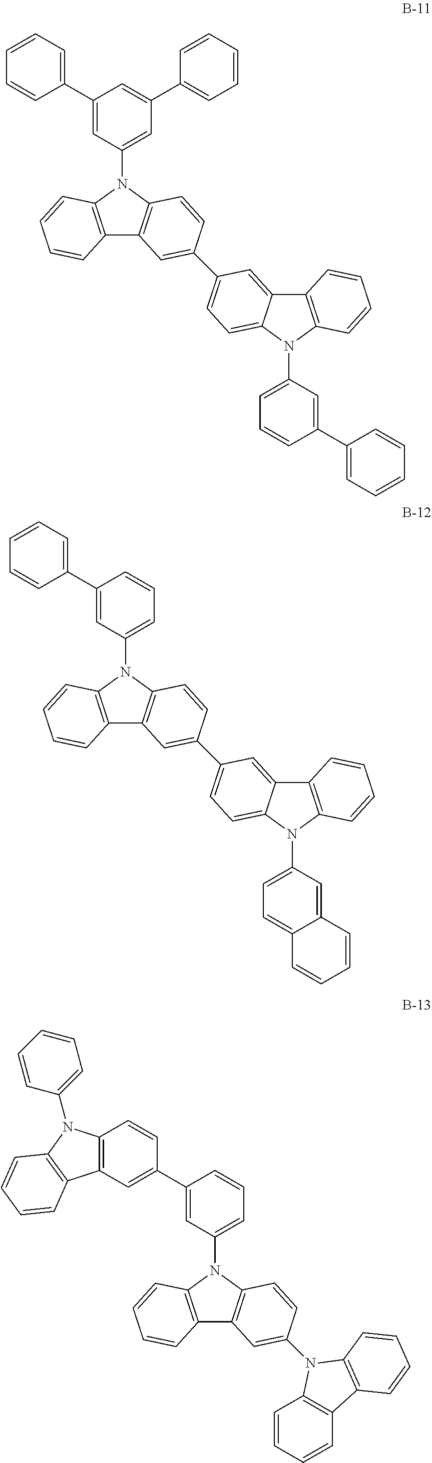 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-n2c3ccccc3c3cc(-c4cccc(-n5c6ccccc6c6cc(-n7c8ccccc8c8ccccc87)ccc65)c4)ccc32)cc1]

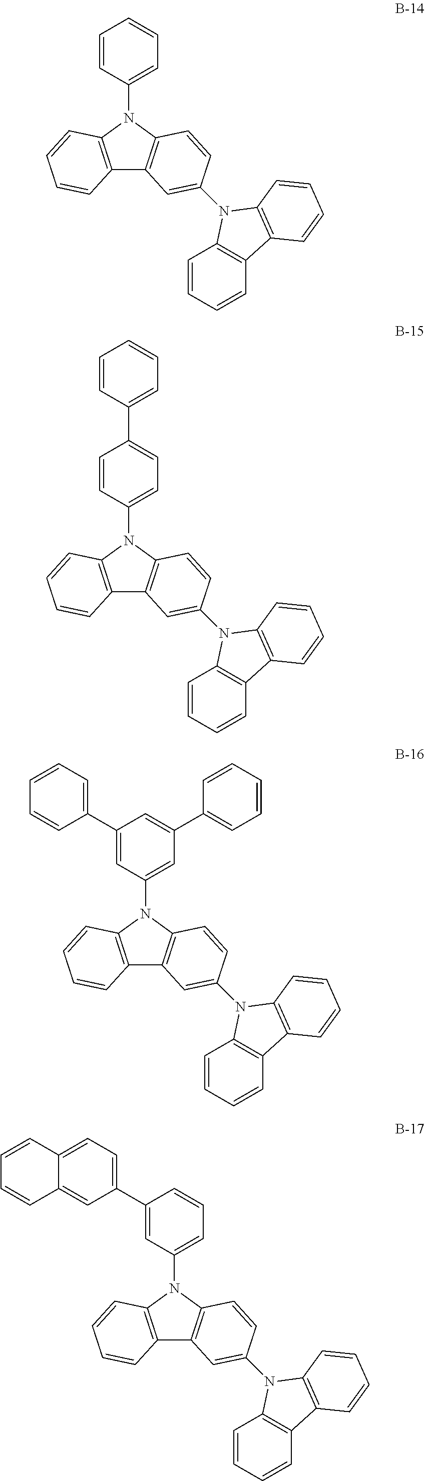

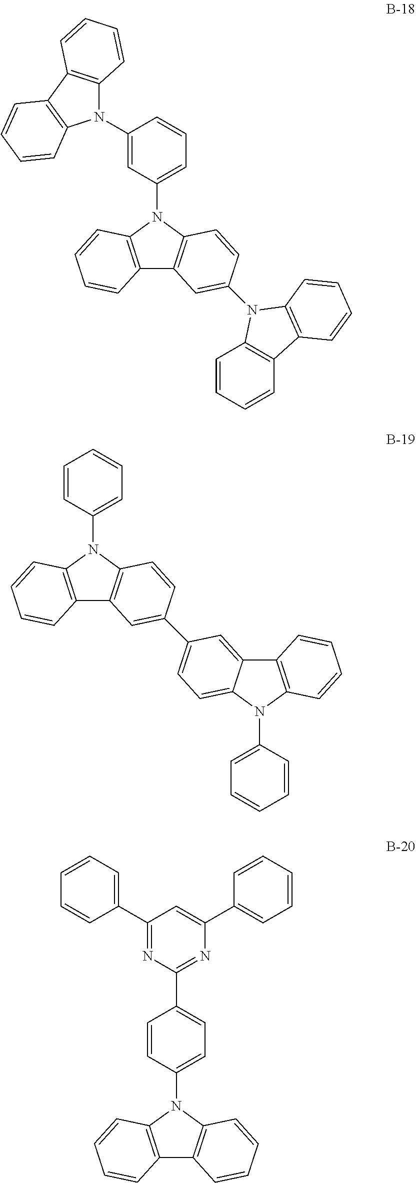

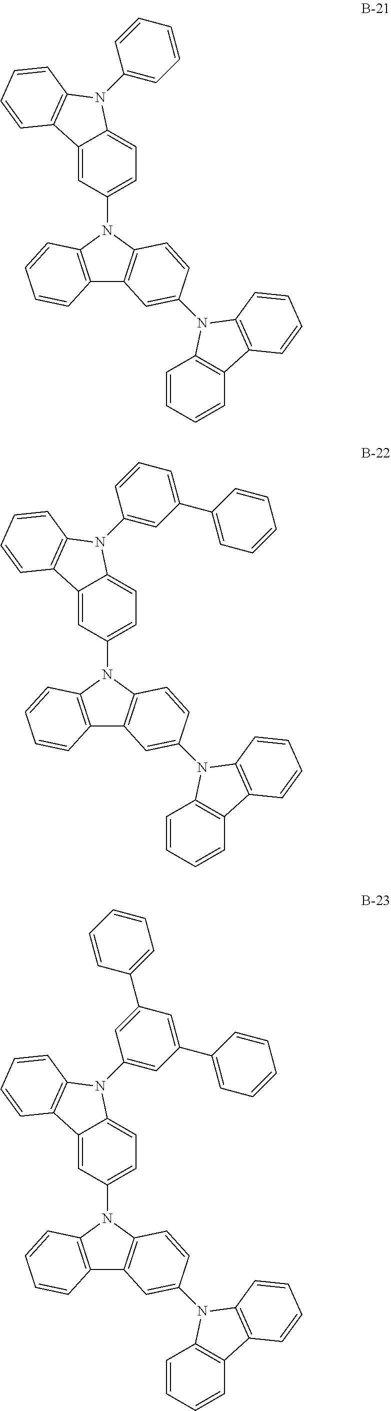 [SMILES: c1ccc(-n2c3ccccc3c3cc(-n4c5ccccc5c5cc(-n6c7ccccc7c7ccccc76)ccc54)ccc32)cc1 ; c1ccc(-c2cccc(-n3c4ccccc4c4cc(-n5c6ccccc6c6cc(-n7c8ccccc8c8ccccc87)ccc65)ccc43)c2)cc1 ; c1ccc(-c2cc(-c3ccccc3)cc(-n3c4ccccc4c4cc(-n5c6ccccc6c6cc(-n7c8ccccc8c8ccccc87)ccc65)ccc43)c2)cc1]

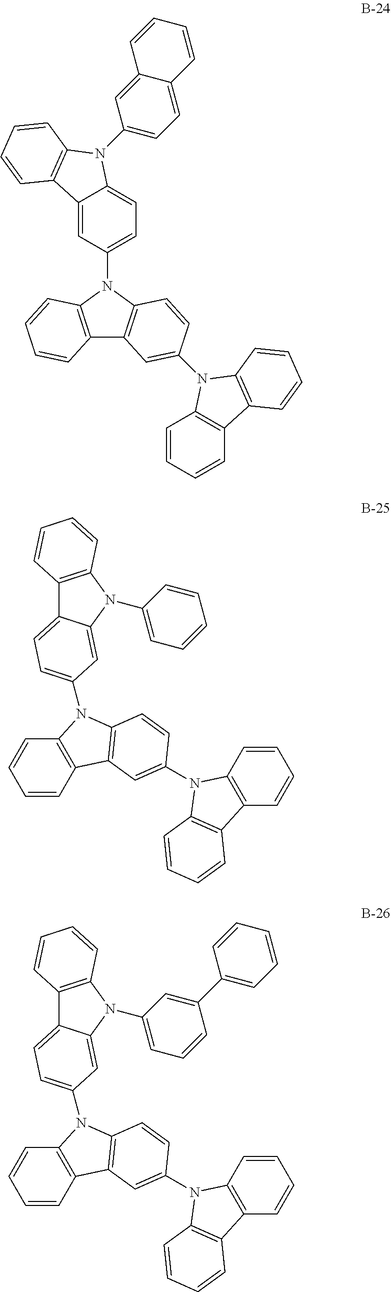 [SMILES: c1ccc2cc(-n3c4ccccc4c4cc(-n5c6ccccc6c6cc(-n7c8ccccc8c8ccccc87)ccc65)ccc43)ccc2c1 ; c1ccc(-n2c3ccccc3c3ccc(-n4c5ccccc5c5cc(-n6c7ccccc7c7ccccc76)ccc54)cc32)cc1 ; c1ccc(-c2cccc(-n3c4ccccc4c4ccc(-n5c6ccccc6c6cc(-n7c8ccccc8c8ccccc87)ccc65)cc43)c2)cc1]

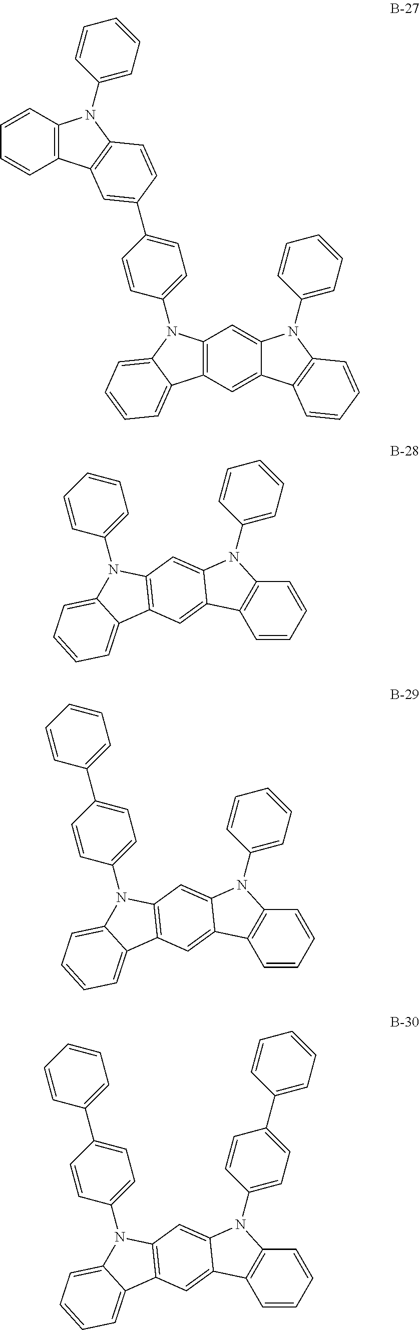

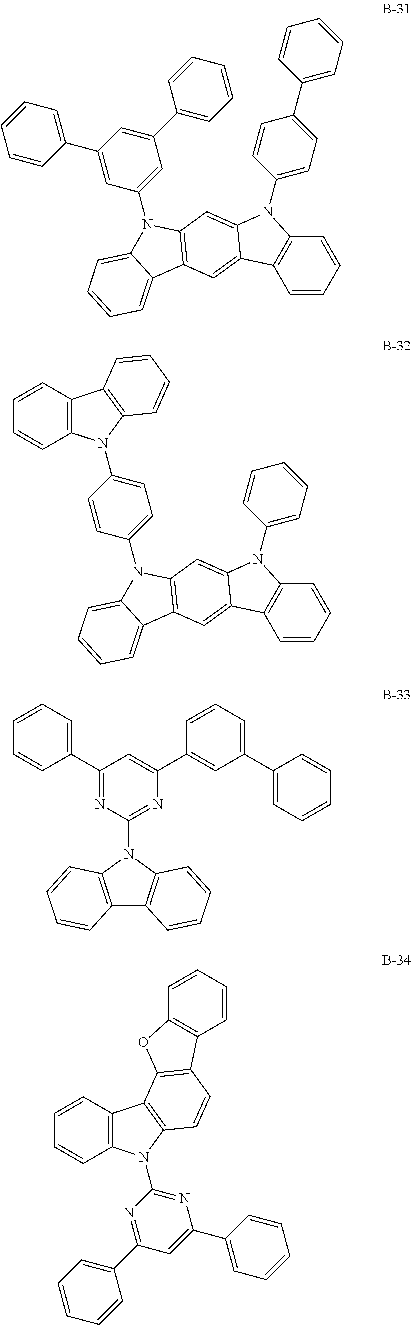

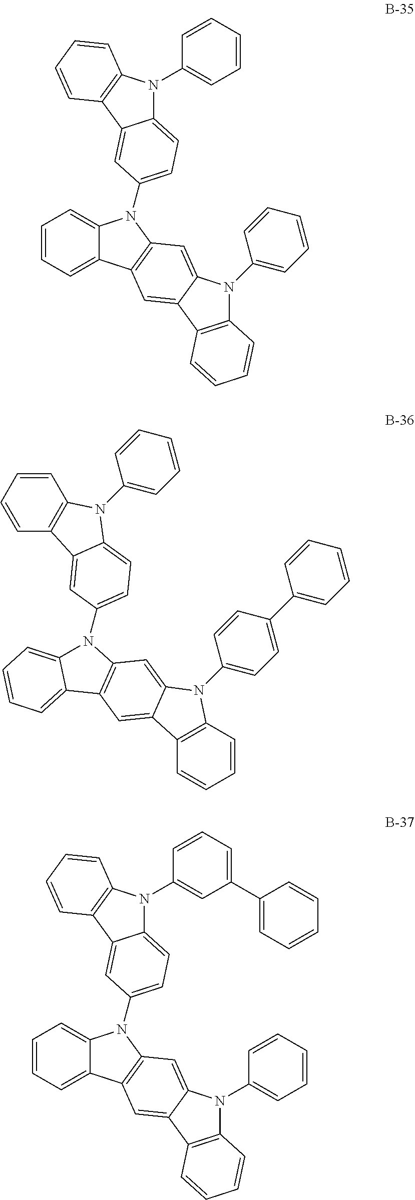

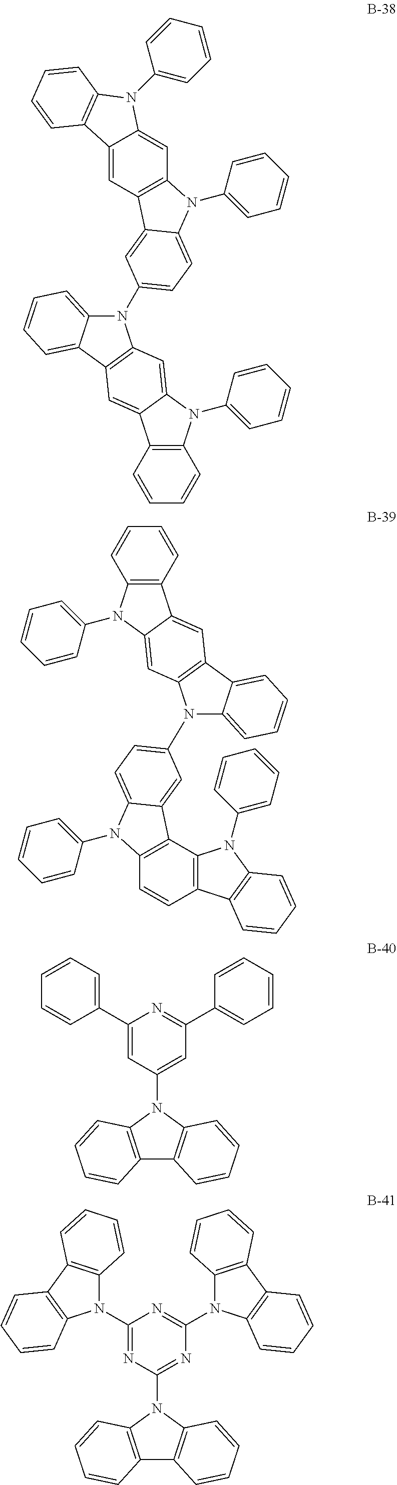

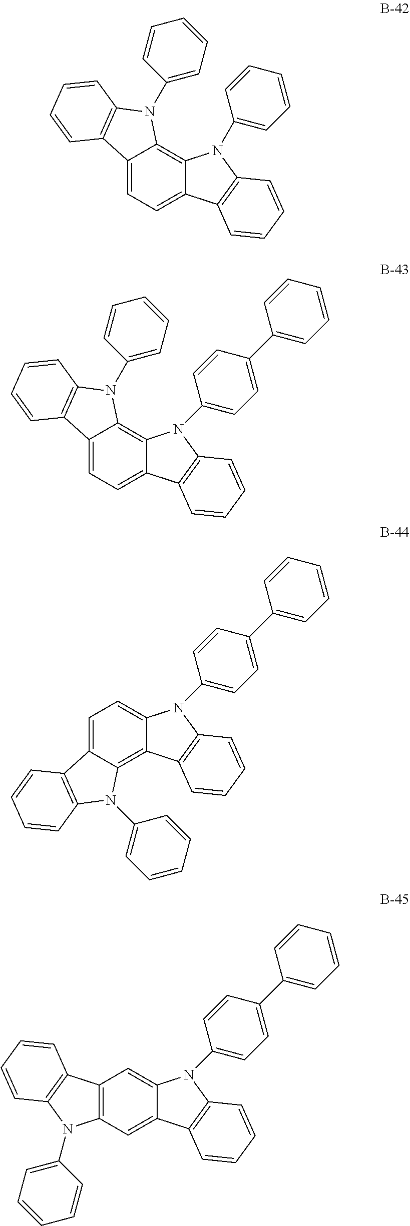 [SMILES: c1ccc(-n2c3ccccc3c3ccc4c5ccccc5n(-c5ccccc5)c4c32)cc1 ; c1ccc(-c2ccc(-n3c4ccccc4c4ccc5c6ccccc6n(-c6ccccc6)c5c43)cc2)cc1 ; c1ccc(-c2ccc(-n3c4ccccc4c4c3ccc3c5ccccc5n(-c5ccccc5)c34)cc2)cc1 ; c1ccc(-c2ccc(-n3c4ccccc4c4cc5c(cc43)c3ccccc3n5-c3ccccc3)cc2)cc1]

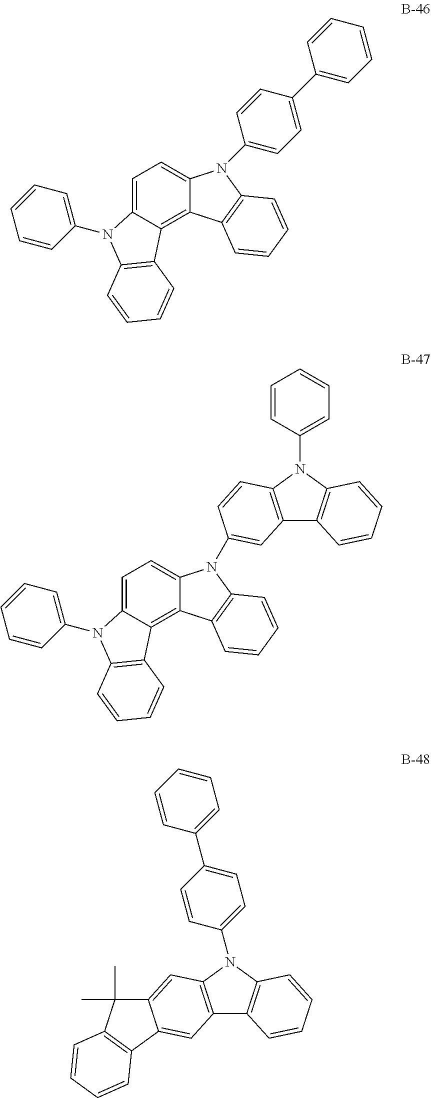 [SMILES: c1ccc(-c2ccc(-n3c4ccccc4c4c5c6ccccc6n(-c6ccccc6)c5ccc43)cc2)cc1 ; c1ccc(-n2c3ccccc3c3cc(-n4c5ccccc5c5c6c7ccccc7n(-c7ccccc7)c6ccc54)ccc32)cc1 ; CC1(C)c2ccccc2-c2cc3c4ccccc4n(-c4ccc(-c5ccccc5)cc4)c3cc21]

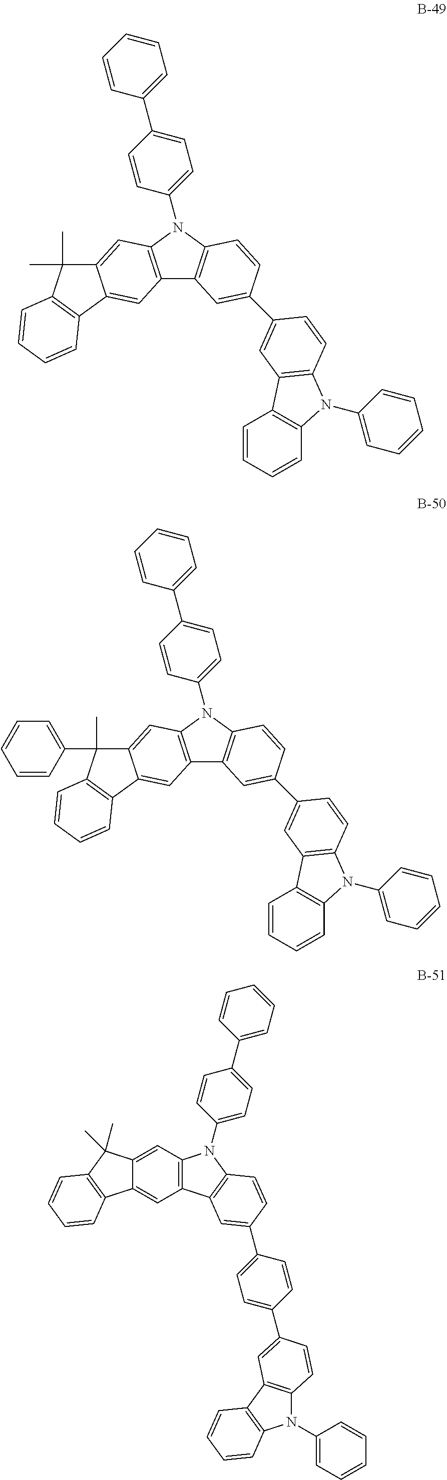 [SMILES: CC1(C)c2ccccc2-c2cc3c4cc(-c5ccc6c(c5)c5ccccc5n6-c5ccccc5)ccc4n(-c4ccc(-c5ccccc5)cc4)c3cc21 ; CC1(c2ccccc2)c2ccccc2-c2cc3c4cc(-c5ccc6c(c5)c5ccccc5n6-c5ccccc5)ccc4n(-c4ccc(-c5ccccc5)cc4)c3cc21 ; CC1(C)c2ccccc2-c2cc3c4cc(-c5ccc(-c6ccc7c(c6)c6ccccc6n7-c6ccccc6)cc5)ccc4n(-c4ccc(-c5ccccc5)cc4)c3cc21]

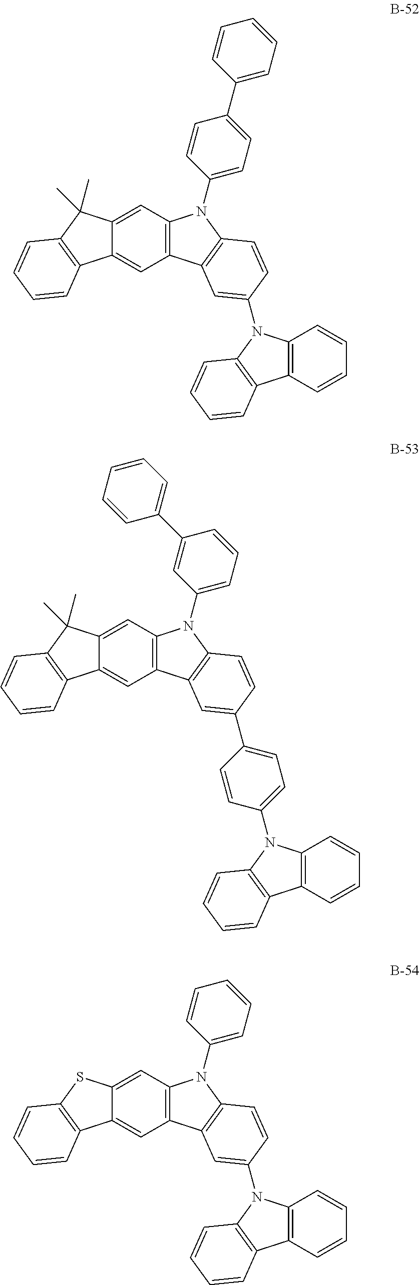 [SMILES: CC1(C)c2ccccc2-c2cc3c4cc(-n5c6ccccc6c6ccccc65)ccc4n(-c4ccc(-c5ccccc5)cc4)c3cc21 ; CC1(C)c2ccccc2-c2cc3c4cc(-c5ccc(-n6c7ccccc7c7ccccc76)cc5)ccc4n(-c4cccc(-c5ccccc5)c4)c3cc21 ; c1ccc(-n2c3ccc(-n4c5ccccc5c5ccccc54)cc3c3cc4c(cc32)sc2ccccc24)cc1]

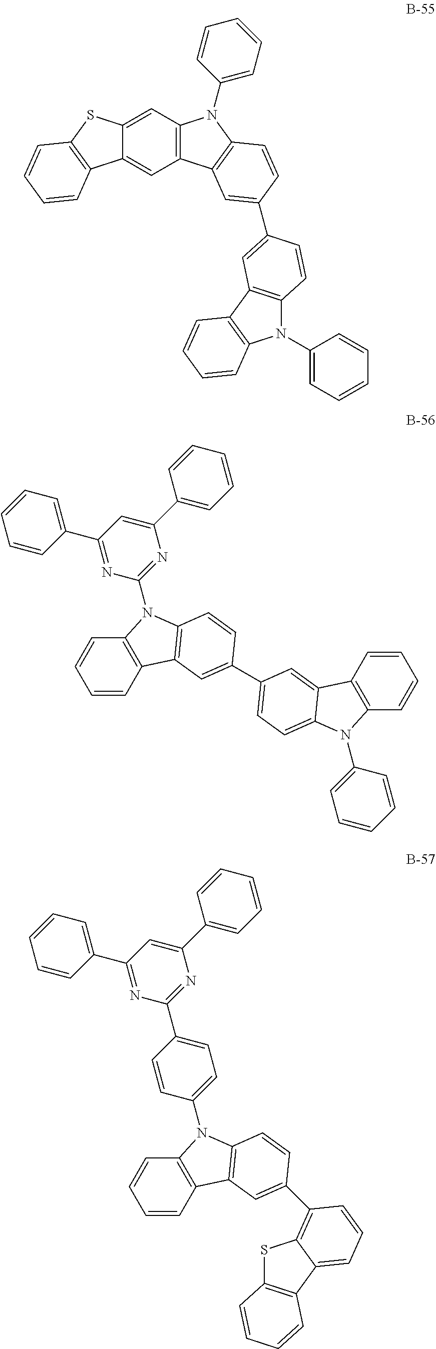 [SMILES: c1ccc(-n2c3ccccc3c3cc(-c4ccc5c(c4)c4cc6c(cc4n5-c4ccccc4)sc4ccccc46)ccc32)cc1 ; c1ccc(-c2cc(-c3ccccc3)nc(-n3c4ccccc4c4cc(-c5ccc6c(c5)c5ccccc5n6-c5ccccc5)ccc43)n2)cc1 ; c1ccc(-c2cc(-c3ccccc3)nc(-c3ccc(-n4c5ccccc5c5cc(-c6cccc7c6sc6ccccc67)ccc54)cc3)n2)cc1]

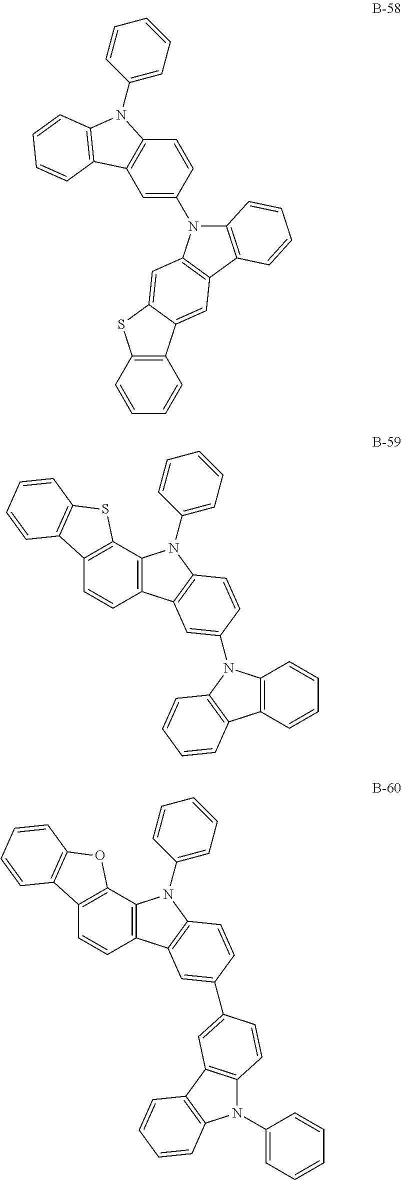

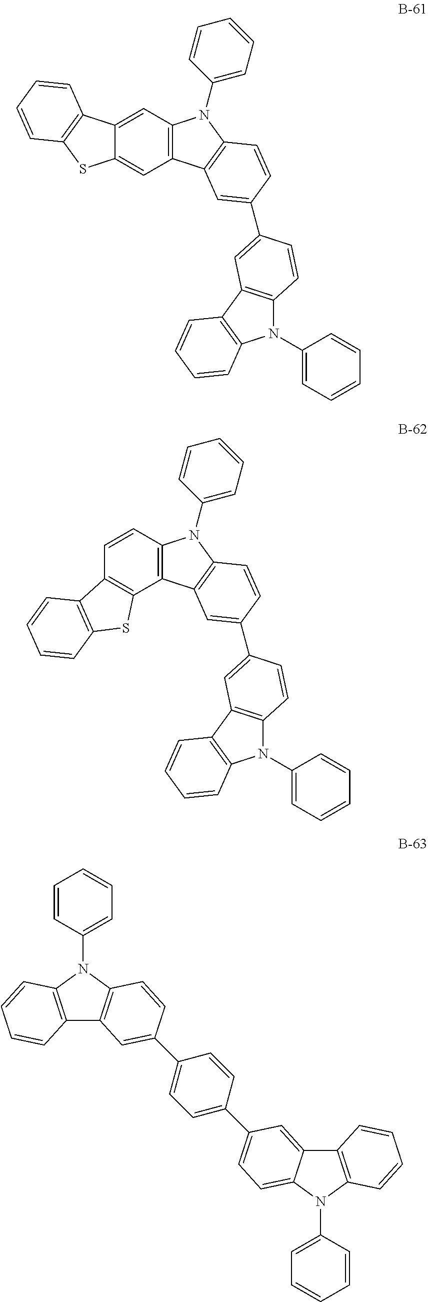 [SMILES: c1ccc(-n2c3ccccc3c3cc(-c4ccc5c(c4)c4cc6sc7ccccc7c6cc4n5-c4ccccc4)ccc32)cc1 ; c1ccc(-n2c3ccccc3c3cc(-c4ccc5c(c4)c4c6sc7ccccc7c6ccc4n5-c4ccccc4)ccc32)cc1 ; c1ccc(-n2c3ccccc3c3cc(-c4ccc(-c5ccc6c(c5)c5ccccc5n6-c5ccccc5)cc4)ccc32)cc1]

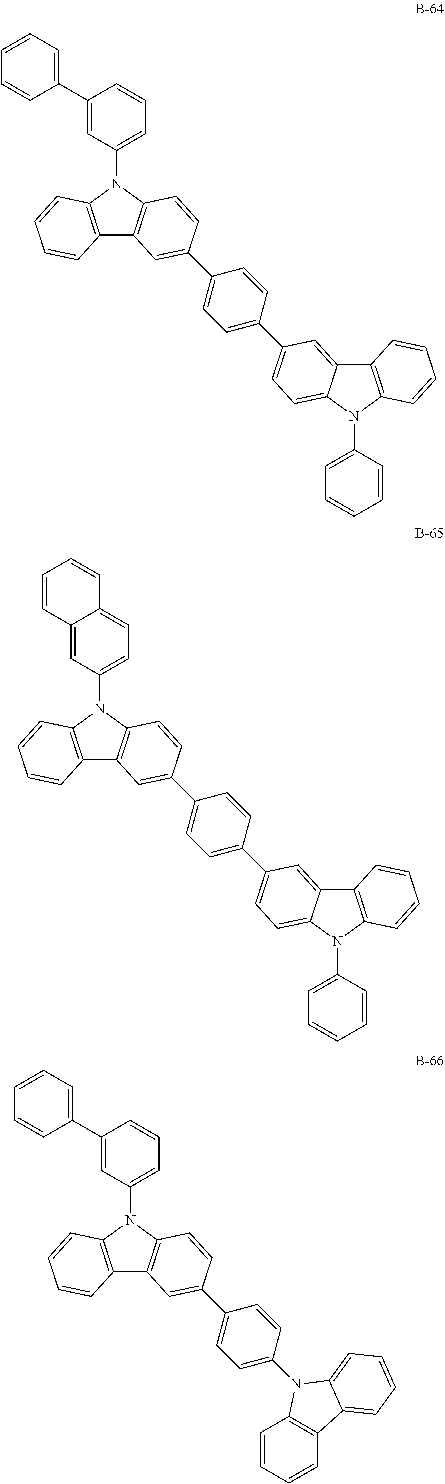 [SMILES: c1ccc(-c2cccc(-n3c4ccccc4c4cc(-c5ccc(-c6ccc7c(c6)c6ccccc6n7-c6ccccc6)cc5)ccc43)c2)cc1 ; c1ccc(-n2c3ccccc3c3cc(-c4ccc(-c5ccc6c(c5)c5ccccc5n6-c5ccc6ccccc6c5)cc4)ccc32)cc1 ; c1ccc(-c2cccc(-n3c4ccccc4c4cc(-c5ccc(-n6c7ccccc7c7ccccc76)cc5)ccc43)c2)cc1]

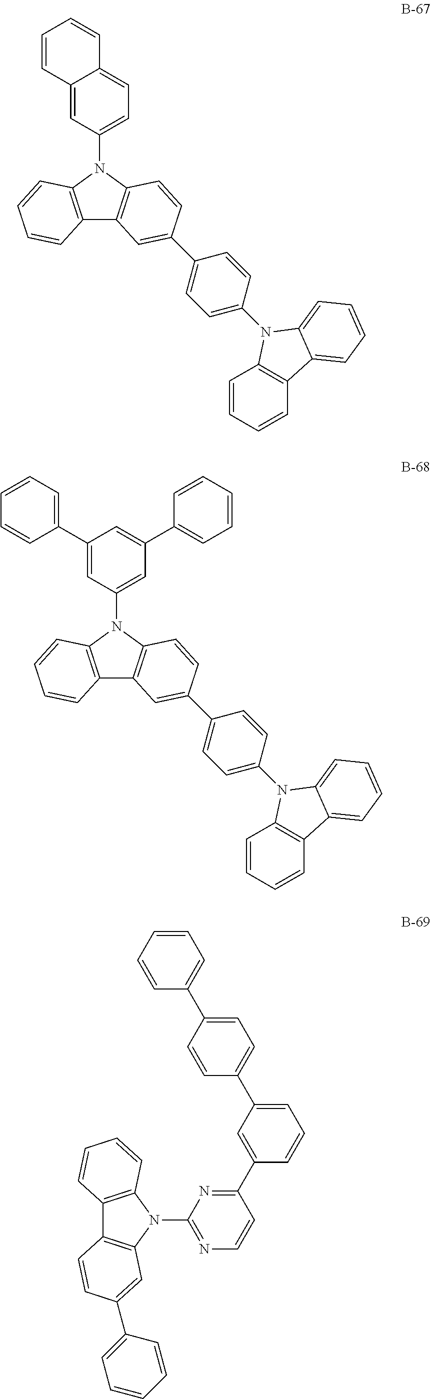 [SMILES: c1ccc2cc(-n3c4ccccc4c4cc(-c5ccc(-n6c7ccccc7c7ccccc76)cc5)ccc43)ccc2c1 ; c1ccc(-c2cc(-c3ccccc3)cc(-n3c4ccccc4c4cc(-c5ccc(-n6c7ccccc7c7ccccc76)cc5)ccc43)c2)cc1 ; c1ccc(-c2ccc(-c3cccc(-c4ccnc(-n5c6ccccc6c6ccc(-c7ccccc7)cc65)n4)c3)cc2)cc1]

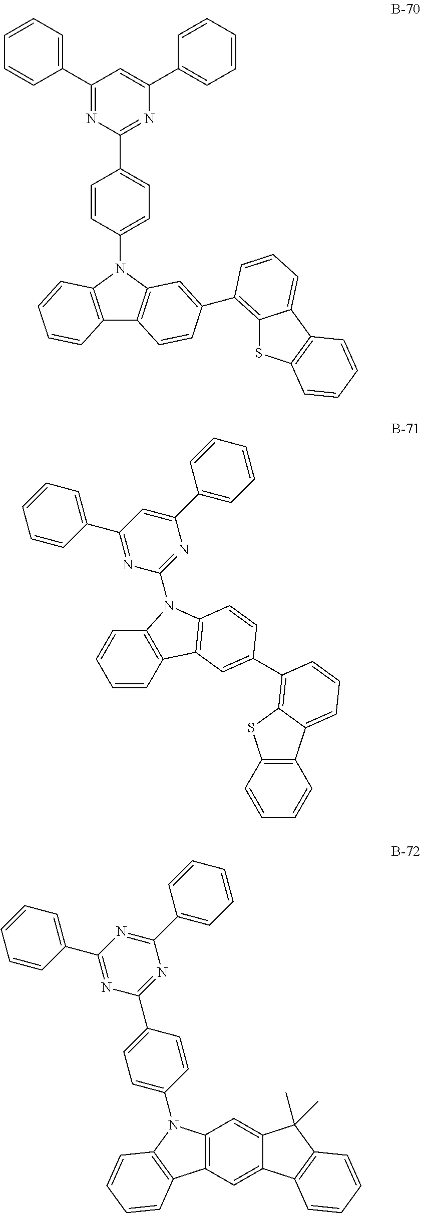 [SMILES: c1ccc(-c2cc(-c3ccccc3)nc(-c3ccc(-n4c5ccccc5c5ccc(-c6cccc7c6sc6ccccc67)cc54)cc3)n2)cc1 ; c1ccc(-c2cc(-c3ccccc3)nc(-n3c4ccccc4c4cc(-c5cccc6c5sc5ccccc56)ccc43)n2)cc1 ; CC1(C)c2ccccc2-c2cc3c4ccccc4n(-c4ccc(-c5nc(-c6ccccc6)nc(-c6ccccc6)n5)cc4)c3cc21]

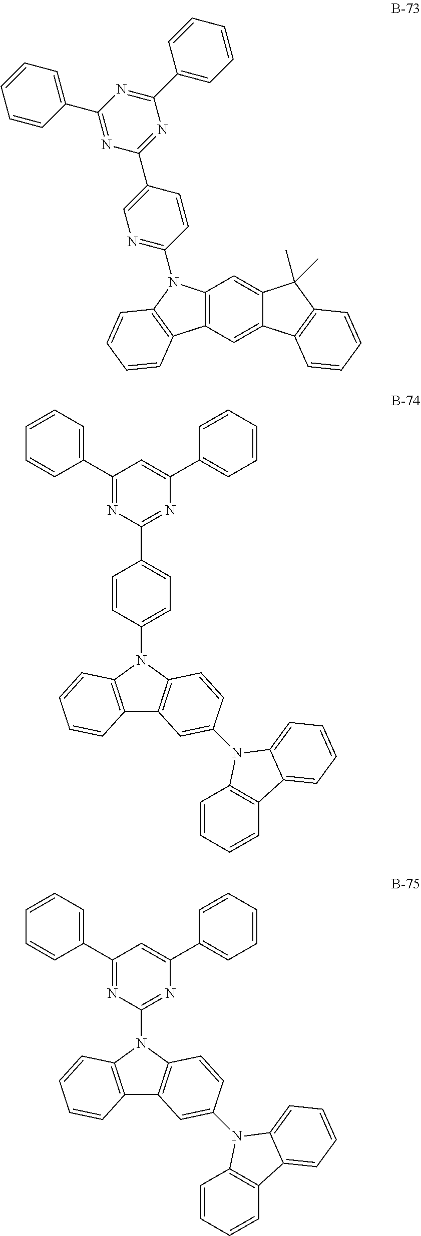

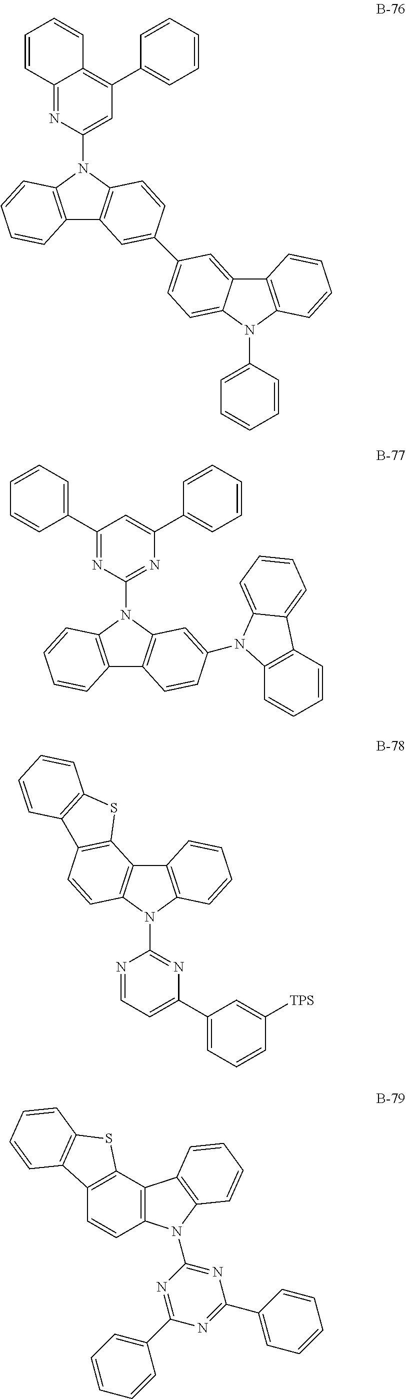

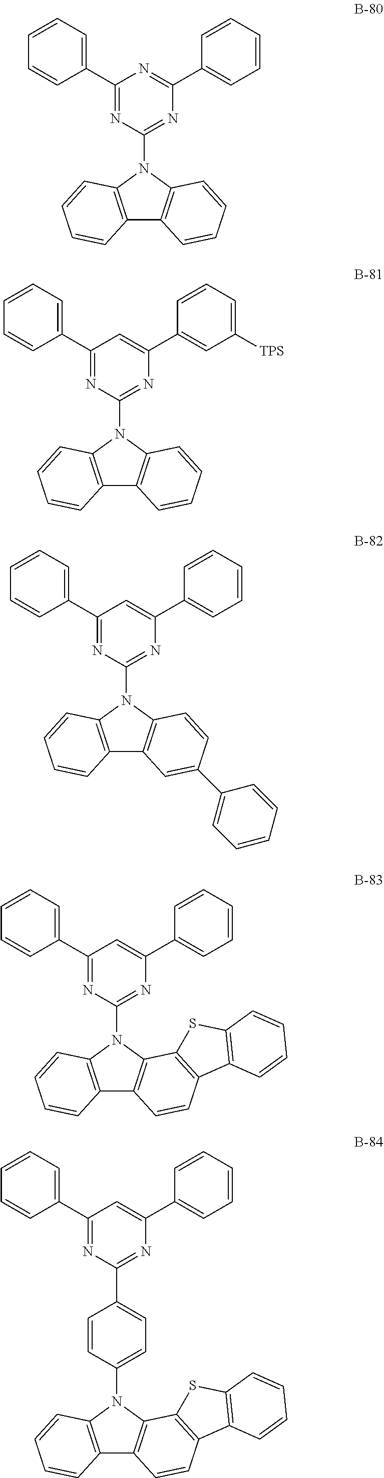

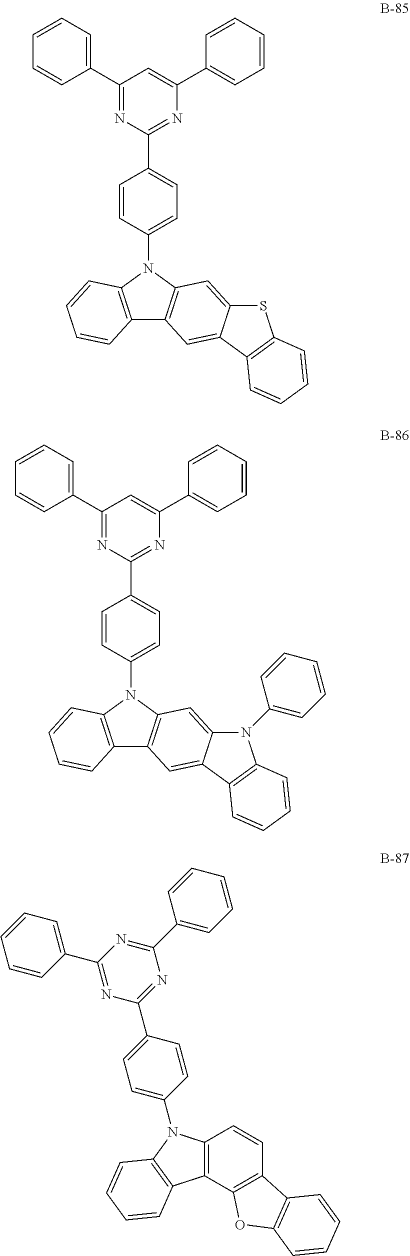 [SMILES: c1ccc(-c2cc(-c3ccccc3)nc(-c3ccc(-n4c5ccccc5c5cc6c(cc54)sc4ccccc46)cc3)n2)cc1 ; c1ccc(-c2cc(-c3ccccc3)nc(-c3ccc(-n4c5ccccc5c5cc6c7ccccc7n(-c7ccccc7)c6cc54)cc3)n2)cc1 ; c1ccc(-c2nc(-c3ccccc3)nc(-c3ccc(-n4c5ccccc5c5c6oc7ccccc7c6ccc54)cc3)n2)cc1]

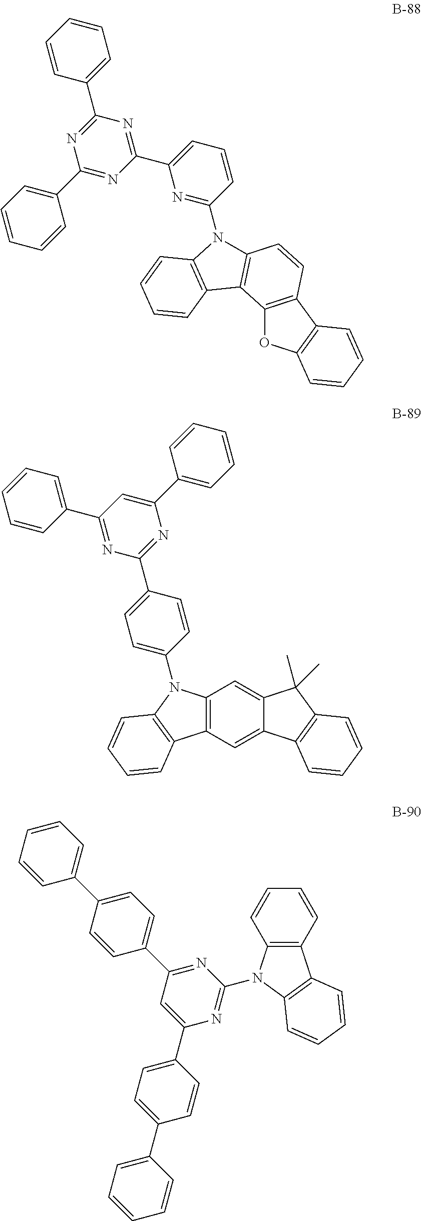 [SMILES: c1ccc(-c2nc(-c3ccccc3)nc(-c3cccc(-n4c5ccccc5c5c6oc7ccccc7c6ccc54)n3)n2)cc1 ; CC1(C)c2ccccc2-c2cc3c4ccccc4n(-c4ccc(-c5nc(-c6ccccc6)cc(-c6ccccc6)n5)cc4)c3cc21 ; c1ccc(-c2ccc(-c3cc(-c4ccc(-c5ccccc5)cc4)nc(-n4c5ccccc5c5ccccc54)n3)cc2)cc1]

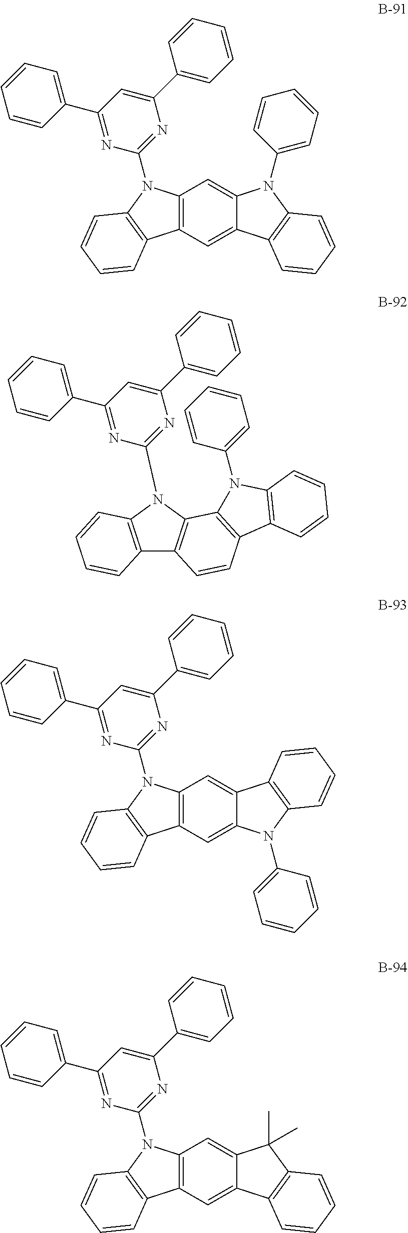

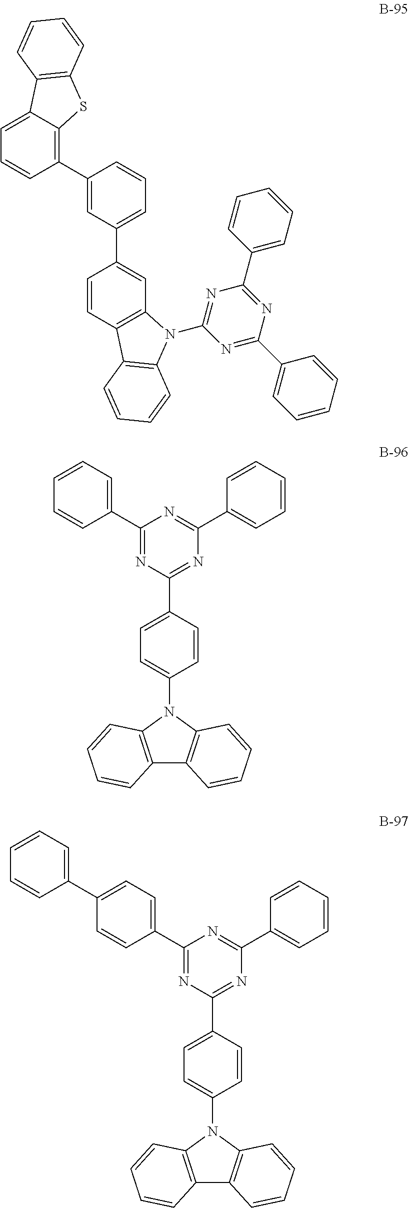 [SMILES: c1ccc(-c2nc(-c3ccccc3)nc(-n3c4ccccc4c4ccc(-c5cccc(-c6cccc7c6sc6ccccc67)c5)cc43)n2)cc1 ; c1ccc(-c2nc(-c3ccccc3)nc(-c3ccc(-n4c5ccccc5c5ccccc54)cc3)n2)cc1 ; c1ccc(-c2ccc(-c3nc(-c4ccccc4)nc(-c4ccc(-n5c6ccccc6c6ccccc65)cc4)n3)cc2)cc1]

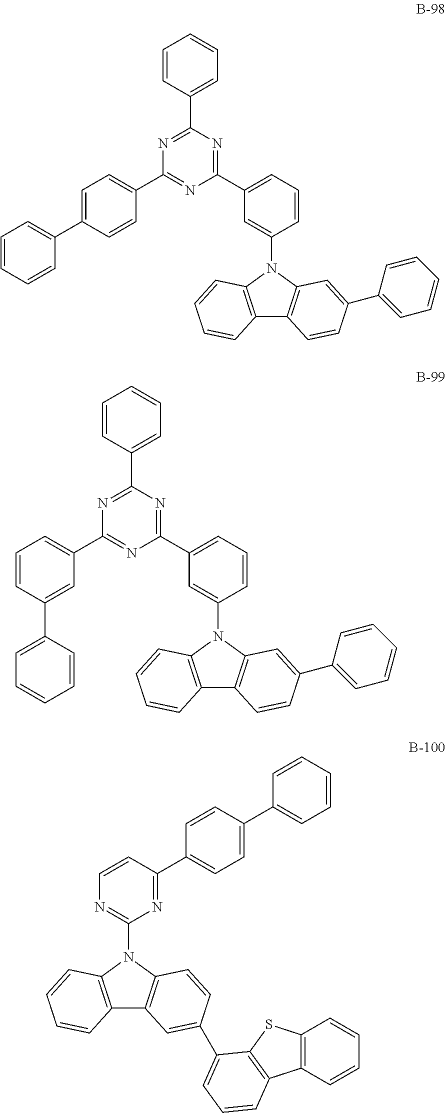 [SMILES: c1ccc(-c2ccc(-c3nc(-c4ccccc4)nc(-c4cccc(-n5c6ccccc6c6ccc(-c7ccccc7)cc65)c4)n3)cc2)cc1 ; c1ccc(-c2cccc(-c3nc(-c4ccccc4)nc(-c4cccc(-n5c6ccccc6c6ccc(-c7ccccc7)cc65)c4)n3)c2)cc1 ; c1ccc(-c2ccc(-c3ccnc(-n4c5ccccc5c5cc(-c6cccc7c6sc6ccccc67)ccc54)n3)cc2)cc1]

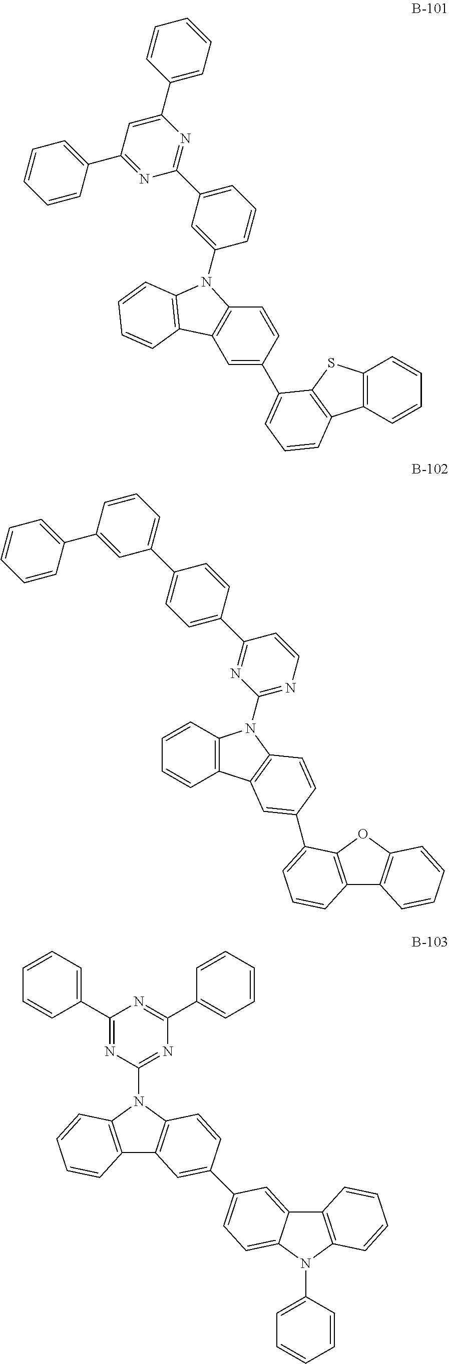 [SMILES: c1ccc(-c2cc(-c3ccccc3)nc(-c3cccc(-n4c5ccccc5c5cc(-c6cccc7c6sc6ccccc67)ccc54)c3)n2)cc1 ; c1ccc(-c2cccc(-c3ccc(-c4ccnc(-n5c6ccccc6c6cc(-c7cccc8c7oc7ccccc78)ccc65)n4)cc3)c2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

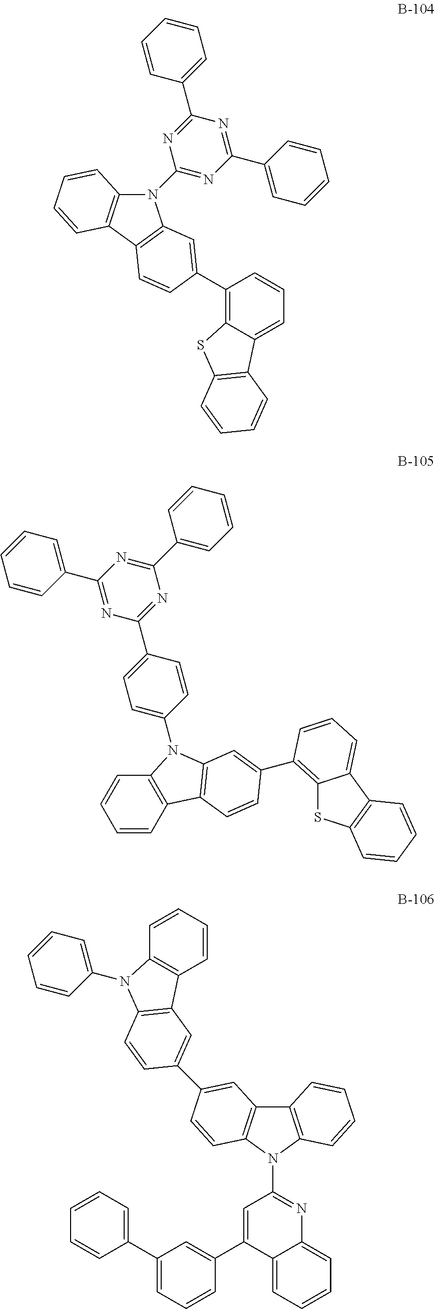 [SMILES: c1ccc(-c2nc(-c3ccccc3)nc(-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)cc3)n2)cc1 ; c1ccc(-c2cccc(-c3cc(-n4c5ccccc5c5cc(-c6ccc7c(c6)c6ccccc6n7-c6ccccc6)ccc54)nc4ccccc34)c2)cc1]

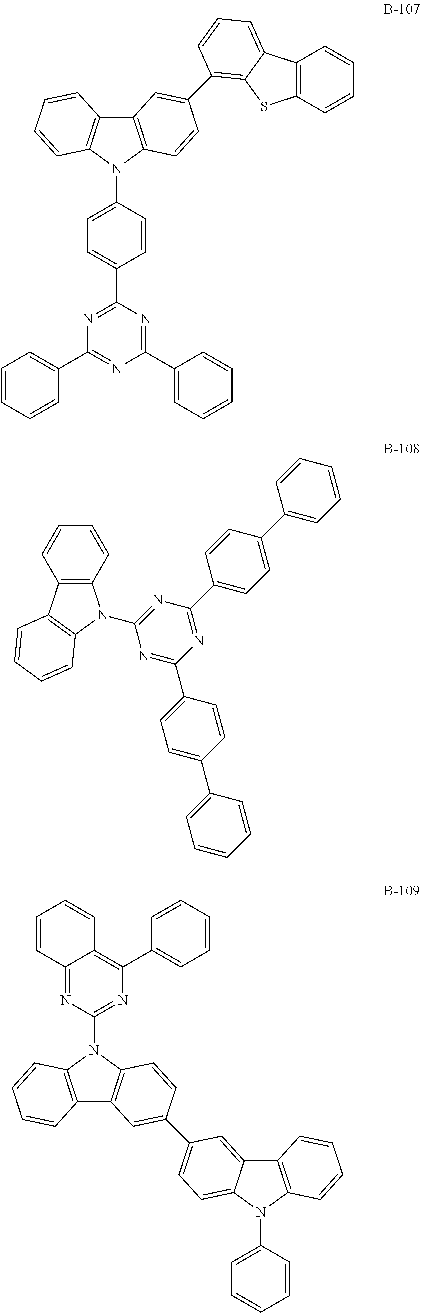 [SMILES: c1ccc(-c2nc(-c3ccccc3)nc(-c3ccc(-n4c5ccccc5c5cc(-c6cccc7c6sc6ccccc67)ccc54)cc3)n2)cc1 ; c1ccc(-c2ccc(-c3nc(-c4ccc(-c5ccccc5)cc4)nc(-n4c5ccccc5c5ccccc54)n3)cc2)cc1 ; c1ccc(-c2nc(-n3c4ccccc4c4cc(-c5ccc6c(c5)c5ccccc5n6-c5ccccc5)ccc43)nc3ccccc23)cc1]

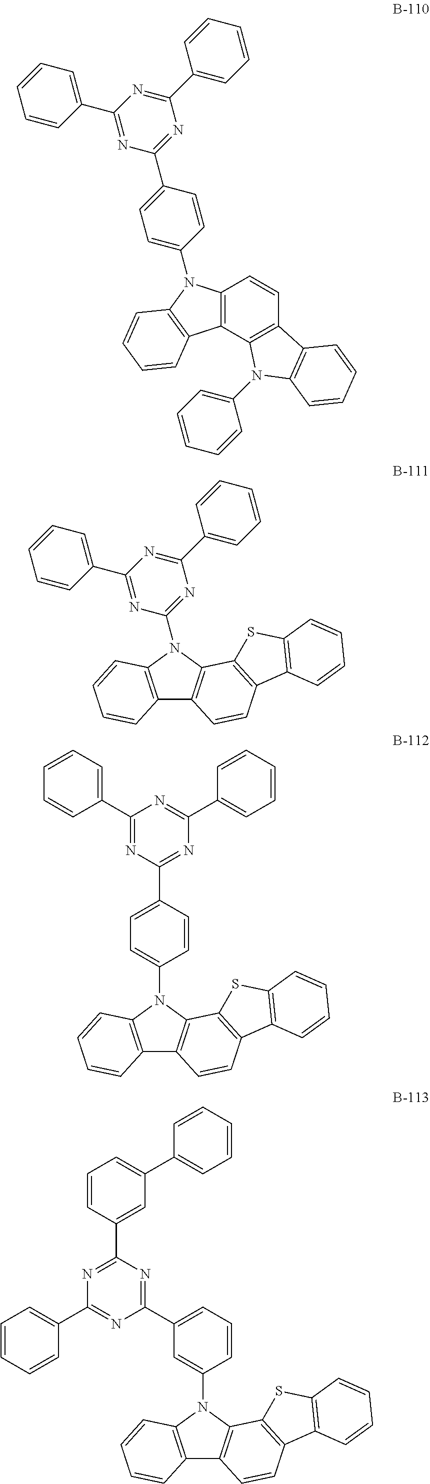

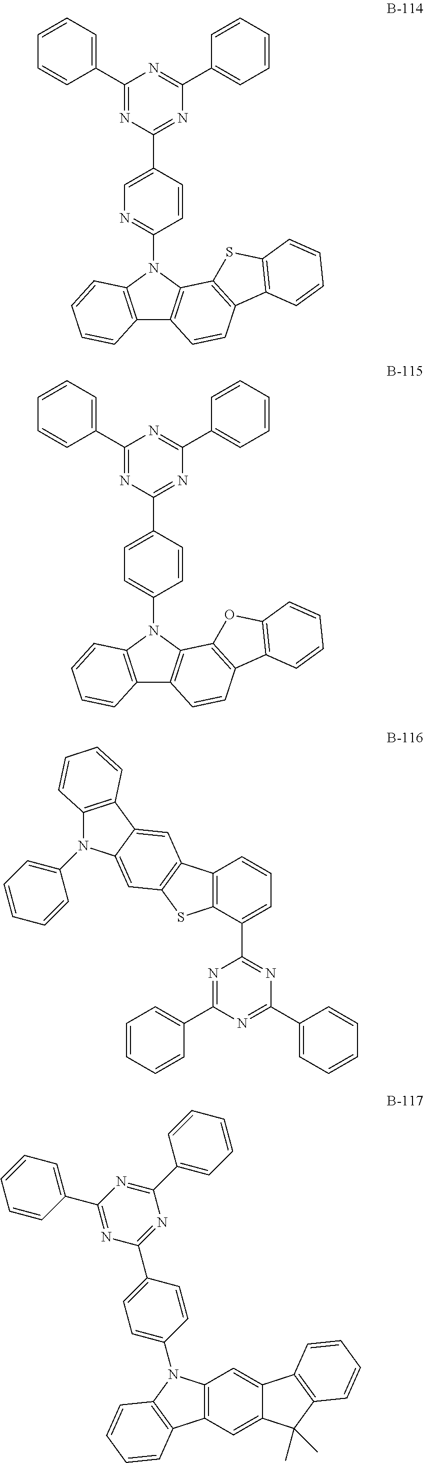

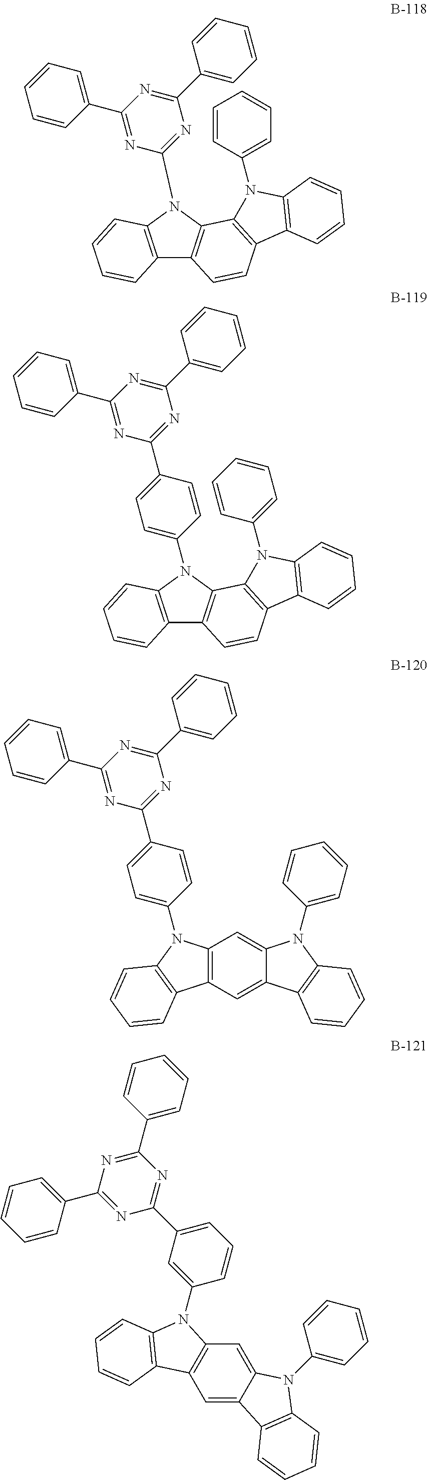

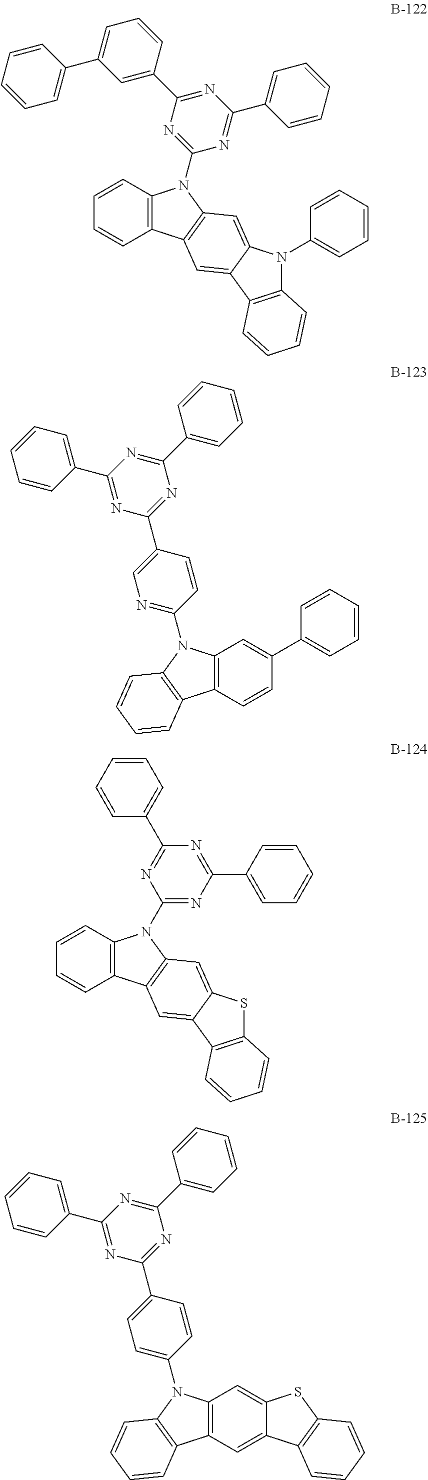

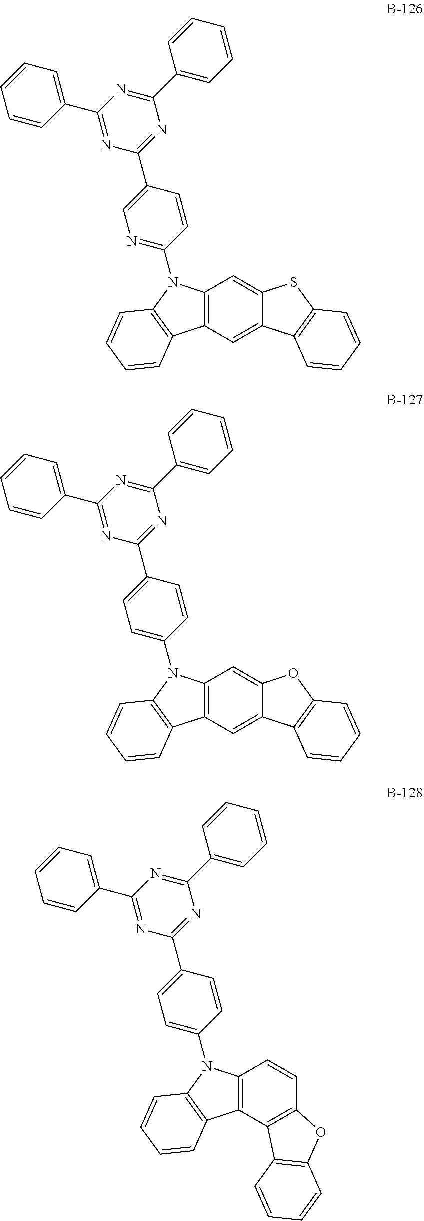 [SMILES: c1ccc(-c2nc(-c3ccccc3)nc(-c3ccc(-n4c5ccccc5c5cc6c(cc54)sc4ccccc46)nc3)n2)cc1 ; c1ccc(-c2nc(-c3ccccc3)nc(-c3ccc(-n4c5ccccc5c5cc6c(cc54)oc4ccccc46)cc3)n2)cc1 ; c1ccc(-c2nc(-c3ccccc3)nc(-c3ccc(-n4c5ccccc5c5c6c(ccc54)oc4ccccc46)cc3)n2)cc1]

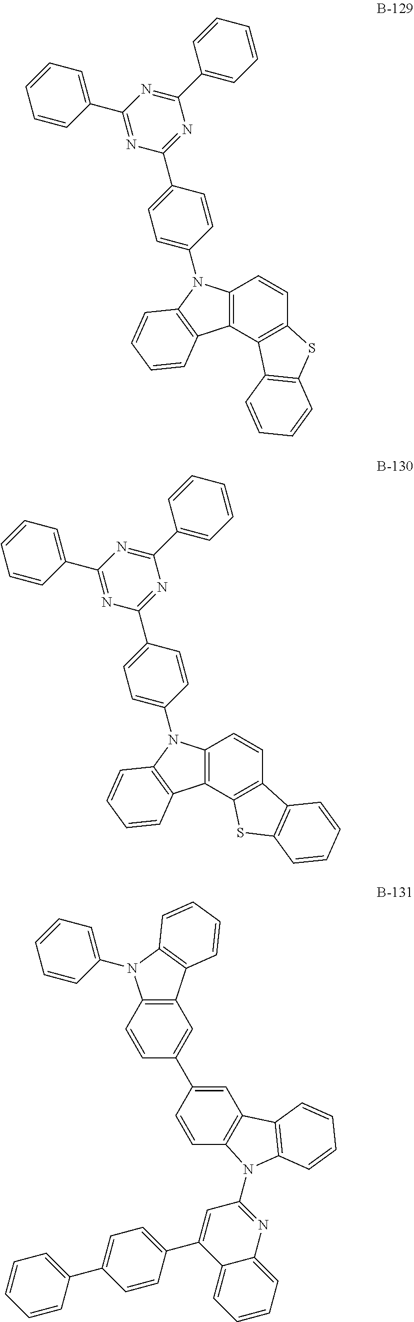 [SMILES: c1ccc(-c2nc(-c3ccccc3)nc(-c3ccc(-n4c5ccccc5c5c6c(ccc54)sc4ccccc46)cc3)n2)cc1 ; c1ccc(-c2nc(-c3ccccc3)nc(-c3ccc(-n4c5ccccc5c5c6sc7ccccc7c6ccc54)cc3)n2)cc1 ; c1ccc(-c2ccc(-c3cc(-n4c5ccccc5c5cc(-c6ccc7c(c6)c6ccccc6n7-c6ccccc6)ccc54)nc4ccccc34)cc2)cc1]

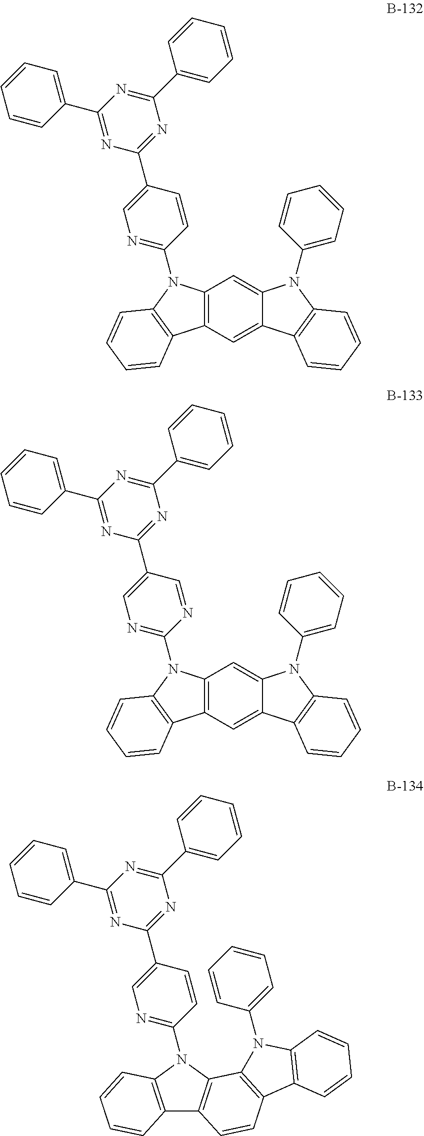 [SMILES: c1ccc(-c2nc(-c3ccccc3)nc(-c3ccc(-n4c5ccccc5c5cc6c7ccccc7n(-c7ccccc7)c6cc54)nc3)n2)cc1 ; c1ccc(-c2nc(-c3ccccc3)nc(-c3cnc(-n4c5ccccc5c5cc6c7ccccc7n(-c7ccccc7)c6cc54)nc3)n2)cc1 ; c1ccc(-c2nc(-c3ccccc3)nc(-c3ccc(-n4c5ccccc5c5ccc6c7ccccc7n(-c7ccccc7)c6c54)nc3)n2)cc1]

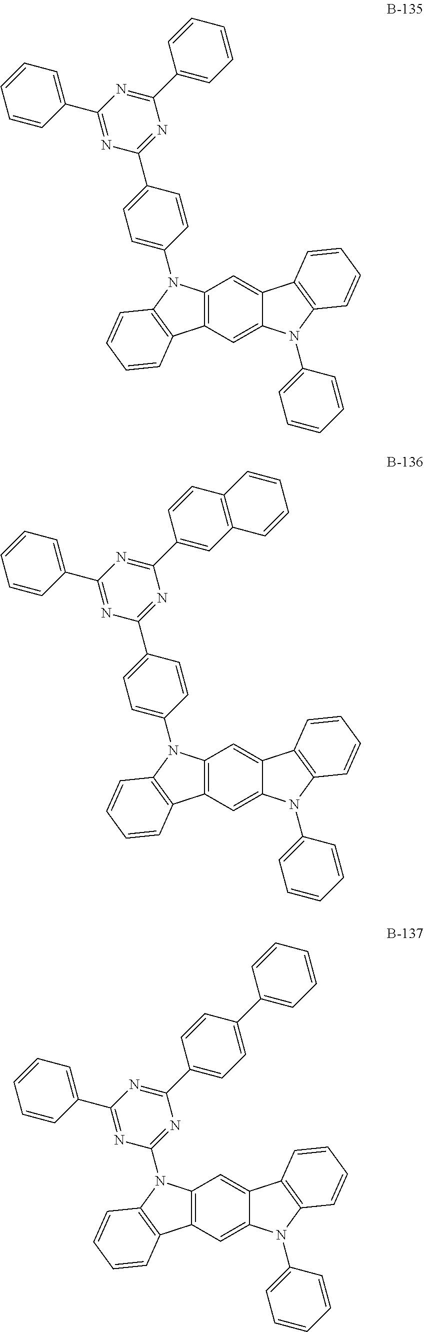 [SMILES: c1ccc(-c2nc(-c3ccccc3)nc(-c3ccc(-n4c5ccccc5c5cc6c(cc54)c4ccccc4n6-c4ccccc4)cc3)n2)cc1 ; c1ccc(-c2nc(-c3ccc(-n4c5ccccc5c5cc6c(cc54)c4ccccc4n6-c4ccccc4)cc3)nc(-c3ccc4ccccc4c3)n2)cc1 ; c1ccc(-c2ccc(-c3nc(-c4ccccc4)nc(-n4c5ccccc5c5cc6c(cc54)c4ccccc4n6-c4ccccc4)n3)cc2)cc1]

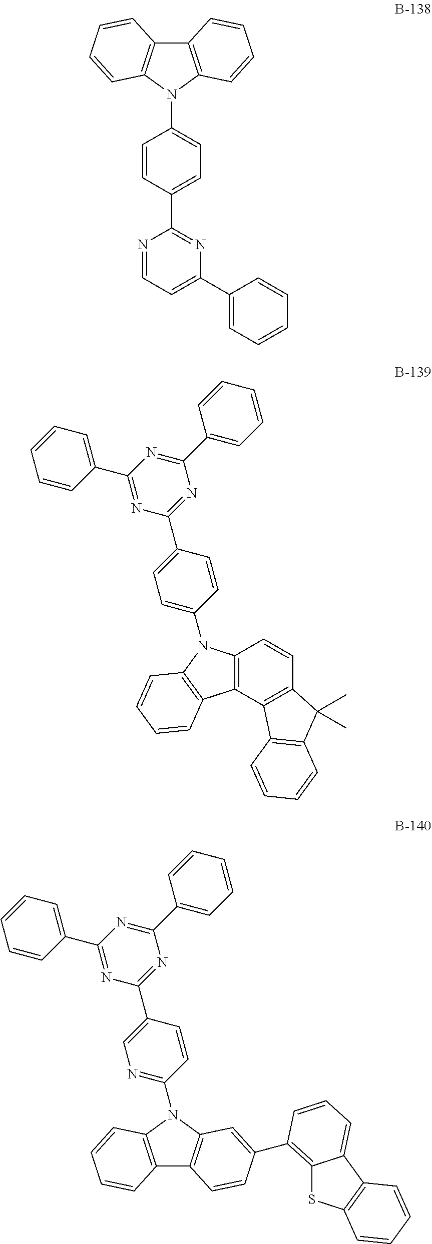

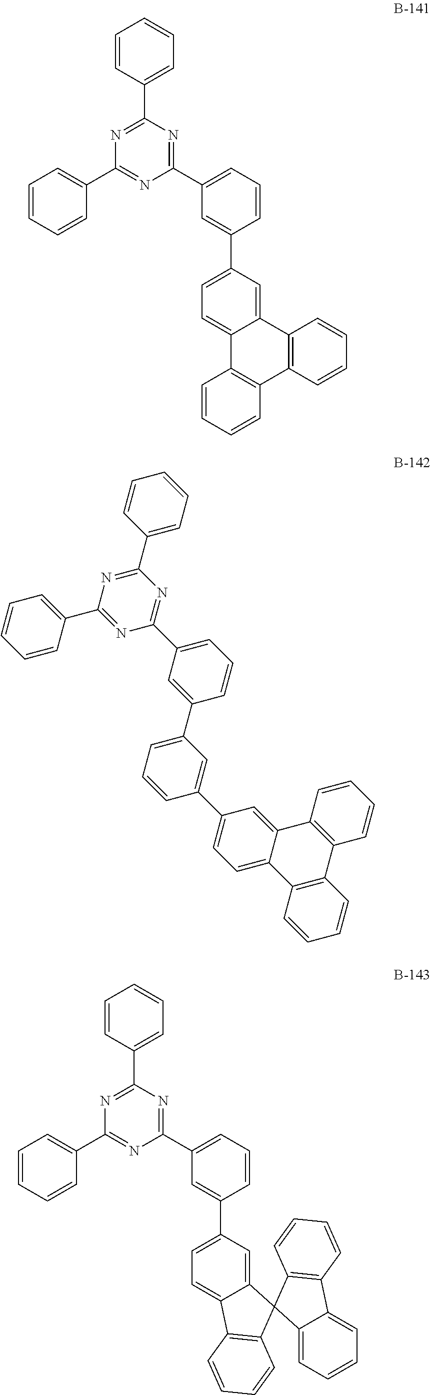 [SMILES: c1ccc(-c2nc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4ccc5c(c4)C4(c6ccccc6-c6ccccc64)c4ccccc4-5)c3)n2)cc1]

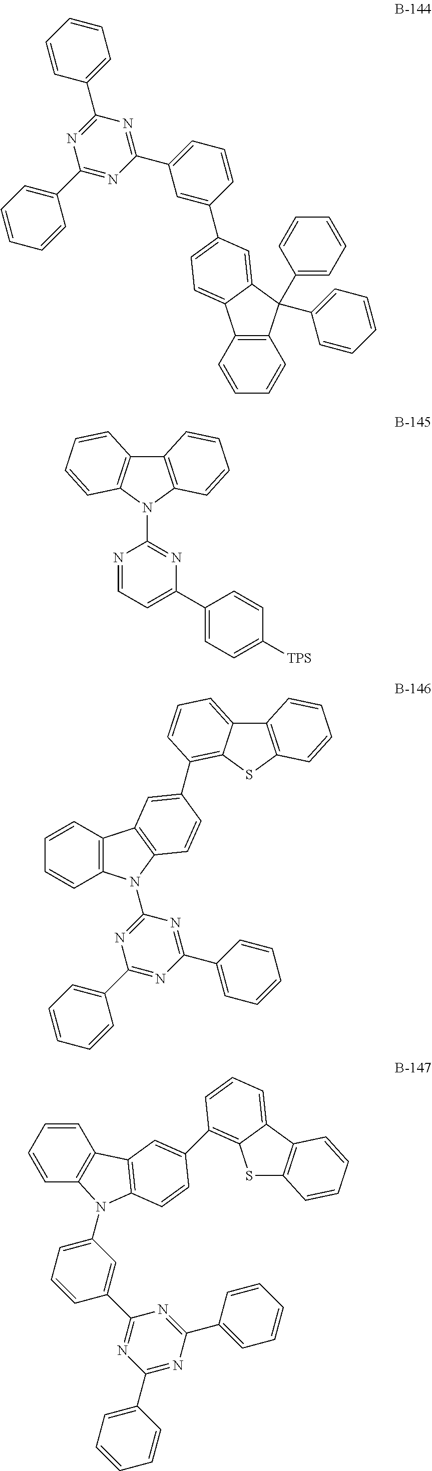

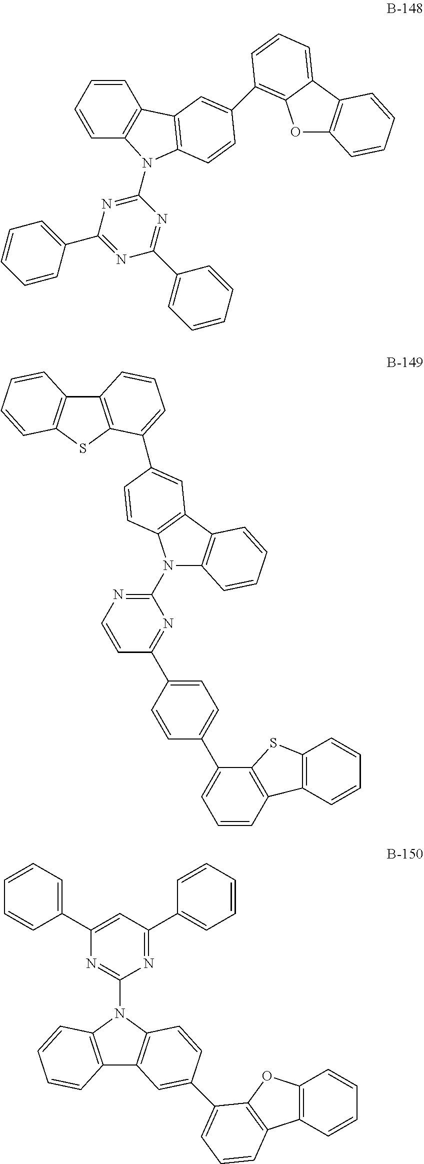

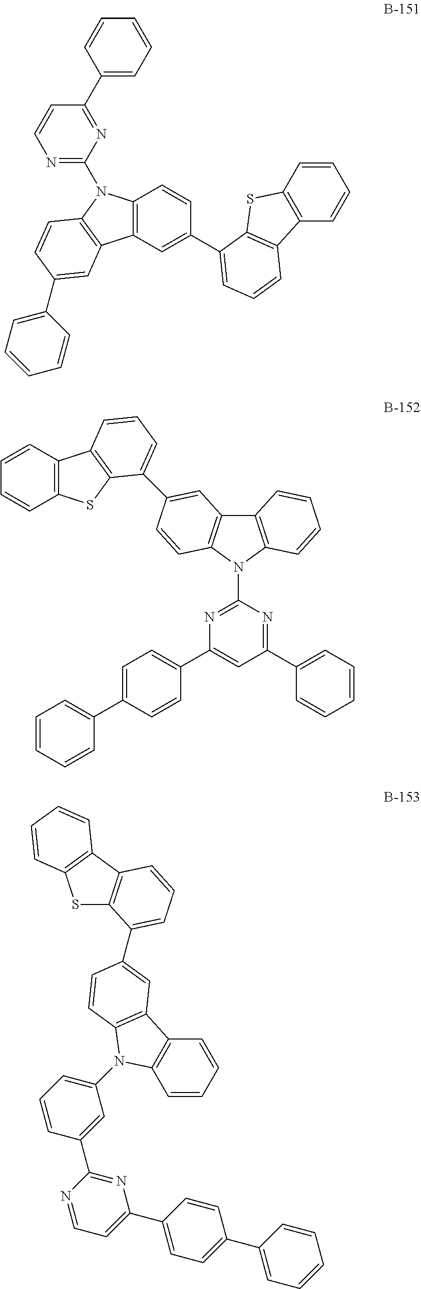

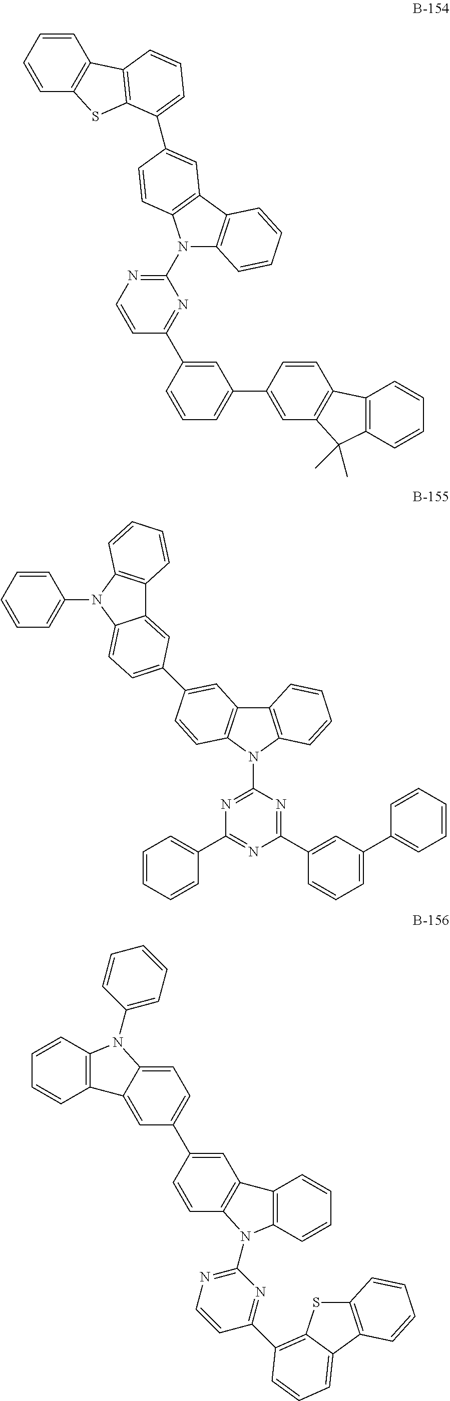 [SMILES: CC1(C)c2ccccc2-c2ccc(-c3cccc(-c4ccnc(-n5c6ccccc6c6cc(-c7cccc8c7sc7ccccc78)ccc65)n4)c3)cc21 ; c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1 ; c1ccc(-n2c3ccccc3c3cc(-c4ccc5c(c4)c4ccccc4n5-c4nccc(-c5cccc6c5sc5ccccc56)n4)ccc32)cc1]

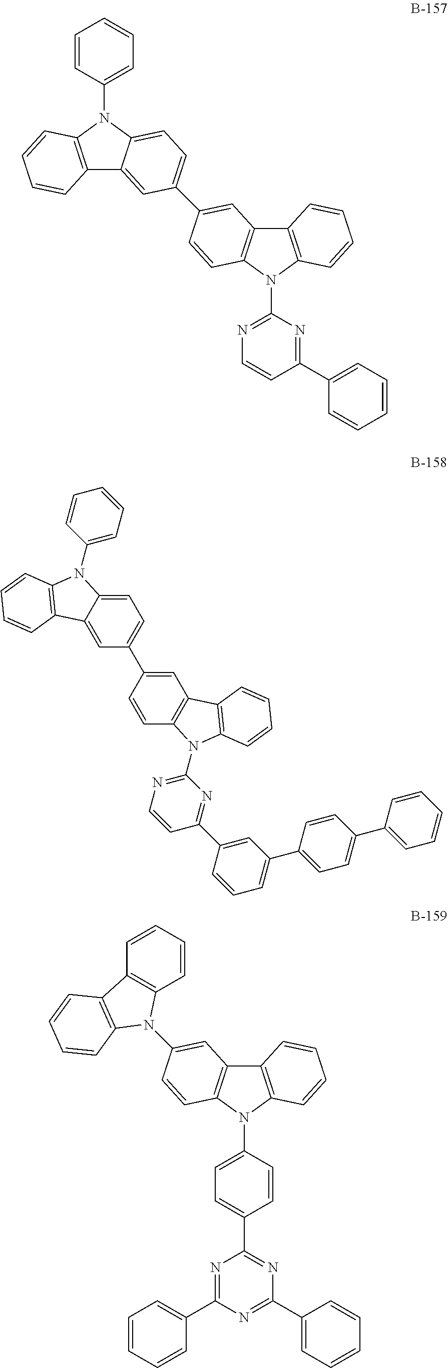 [SMILES: c1ccc(-c2ccnc(-n3c4ccccc4c4cc(-c5ccc6c(c5)c5ccccc5n6-c5ccccc5)ccc43)n2)cc1 ; c1ccc(-c2ccc(-c3cccc(-c4ccnc(-n5c6ccccc6c6cc(-c7ccc8c(c7)c7ccccc7n8-c7ccccc7)ccc65)n4)c3)cc2)cc1 ; c1ccc(-c2nc(-c3ccccc3)nc(-c3ccc(-n4c5ccccc5c5cc(-n6c7ccccc7c7ccccc76)ccc54)cc3)n2)cc1]

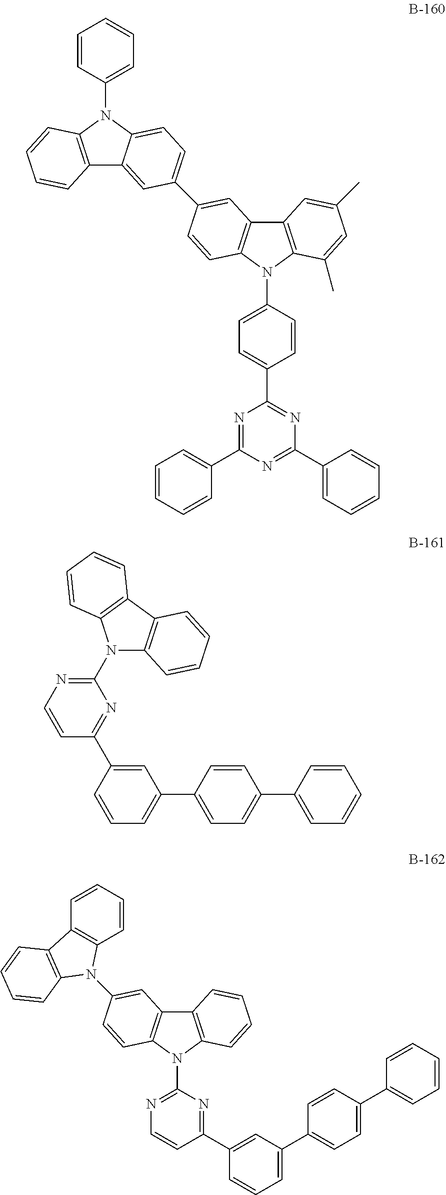 [SMILES: Cc1cc(C)c2c(c1)c1cc(-c3ccc4c(c3)c3ccccc3n4-c3ccccc3)ccc1n2-c1ccc(-c2nc(-c3ccccc3)nc(-c3ccccc3)n2)cc1 ; c1ccc(-c2ccc(-c3cccc(-c4ccnc(-n5c6ccccc6c6ccccc65)n4)c3)cc2)cc1 ; c1ccc(-c2ccc(-c3cccc(-c4ccnc(-n5c6ccccc6c6cc(-n7c8ccccc8c8ccccc87)ccc65)n4)c3)cc2)cc1]

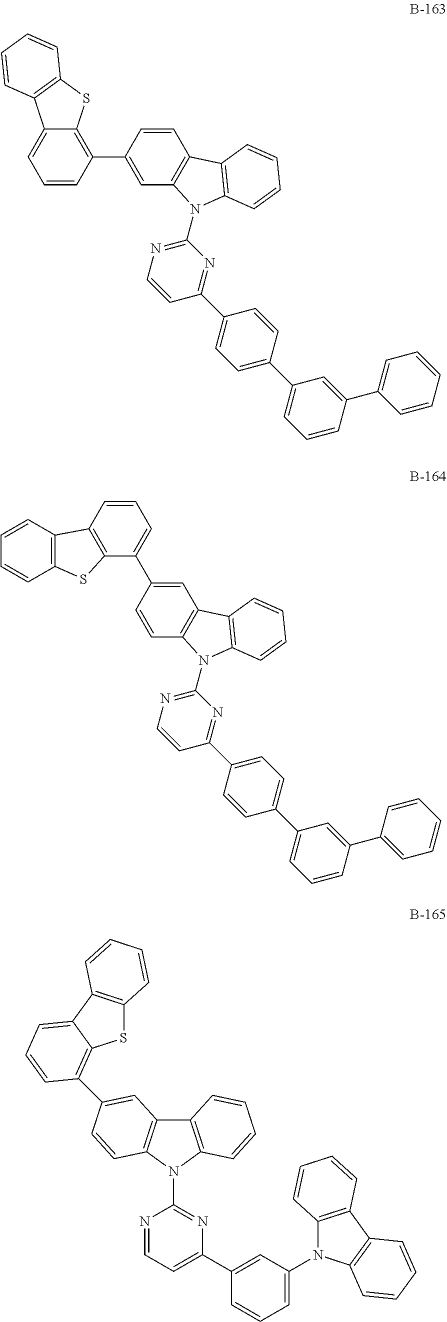 [SMILES: c1ccc(-c2cccc(-c3ccc(-c4ccnc(-n5c6ccccc6c6ccc(-c7cccc8c7sc7ccccc78)cc65)n4)cc3)c2)cc1 ; c1ccc(-c2cccc(-c3ccc(-c4ccnc(-n5c6ccccc6c6cc(-c7cccc8c7sc7ccccc78)ccc65)n4)cc3)c2)cc1 ; c1cc(-c2ccnc(-n3c4ccccc4c4cc(-c5cccc6c5sc5ccccc56)ccc43)n2)cc(-n2c3ccccc3c3ccccc32)c1]

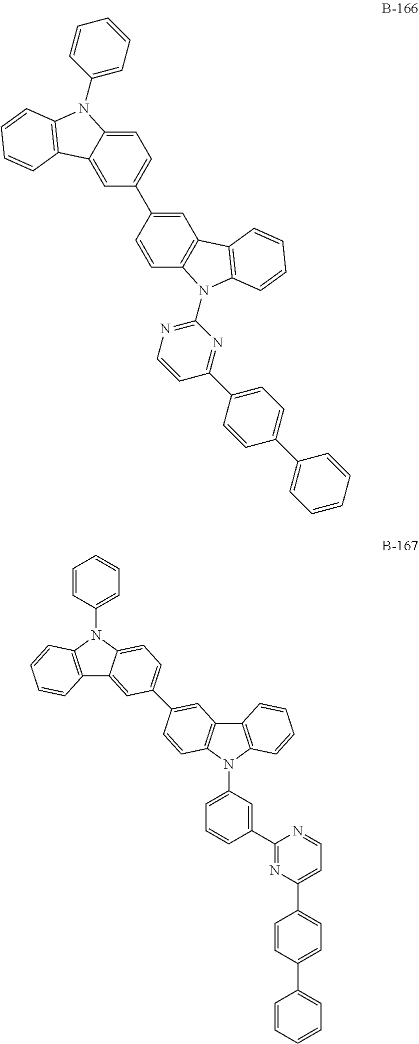 [SMILES: c1ccc(-c2ccc(-c3ccnc(-n4c5ccccc5c5cc(-c6ccc7c(c6)c6ccccc6n7-c6ccccc6)ccc54)n3)cc2)cc1 ; c1ccc(-c2ccc(-c3ccnc(-c4cccc(-n5c6ccccc6c6cc(-c7ccc8c(c7)c7ccccc7n8-c7ccccc7)ccc65)c4)n3)cc2)cc1]

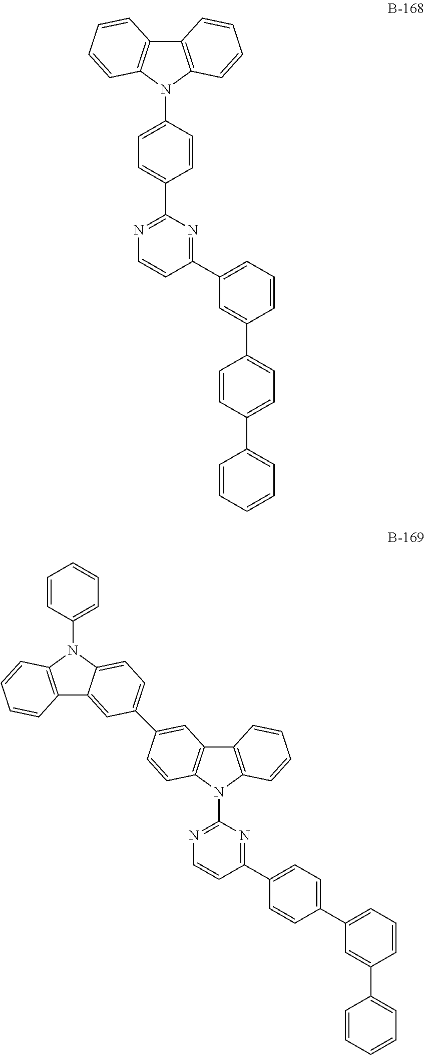 [SMILES: c1ccc(-c2ccc(-c3cccc(-c4ccnc(-c5ccc(-n6c7ccccc7c7ccccc76)cc5)n4)c3)cc2)cc1 ; c1ccc(-c2cccc(-c3ccc(-c4ccnc(-n5c6ccccc6c6cc(-c7ccc8c(c7)c7ccccc7n8-c7ccccc7)ccc65)n4)cc3)c2)cc1]

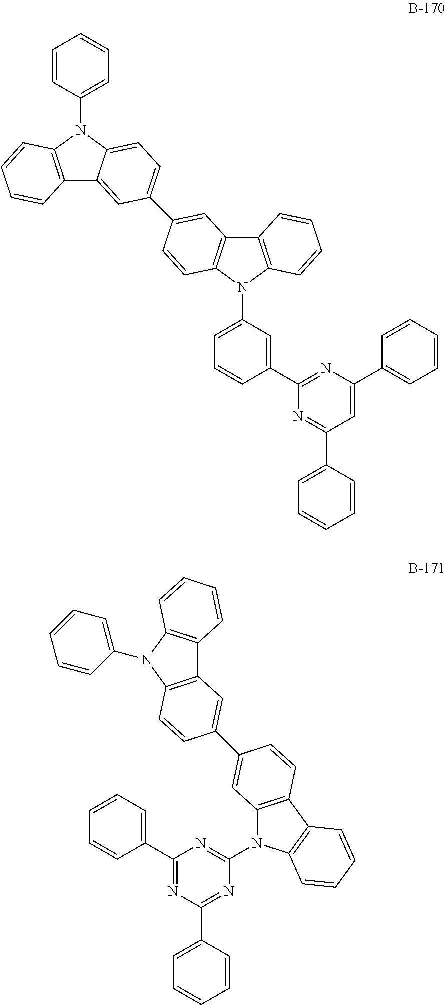 [SMILES: c1ccc(-c2cc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-n3c4ccccc4c4ccc(-c5ccc6c(c5)c5ccccc5n6-c5ccccc5)cc43)n2)cc1]

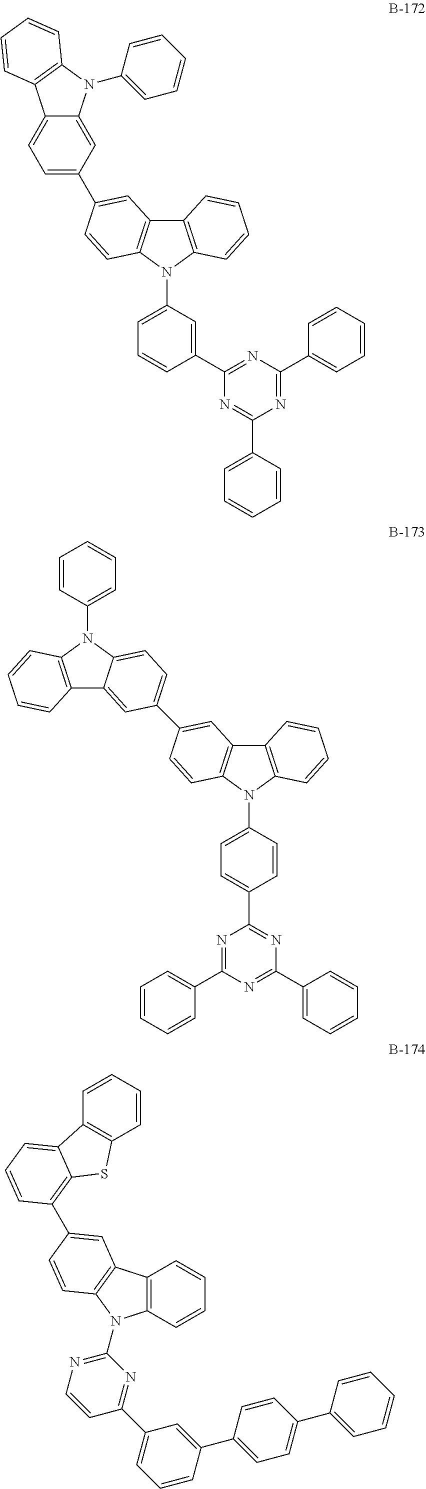 [SMILES: c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2ccc(-c3cccc(-c4ccnc(-n5c6ccccc6c6cc(-c7cccc8c7sc7ccccc78)ccc65)n4)c3)cc2)cc1]

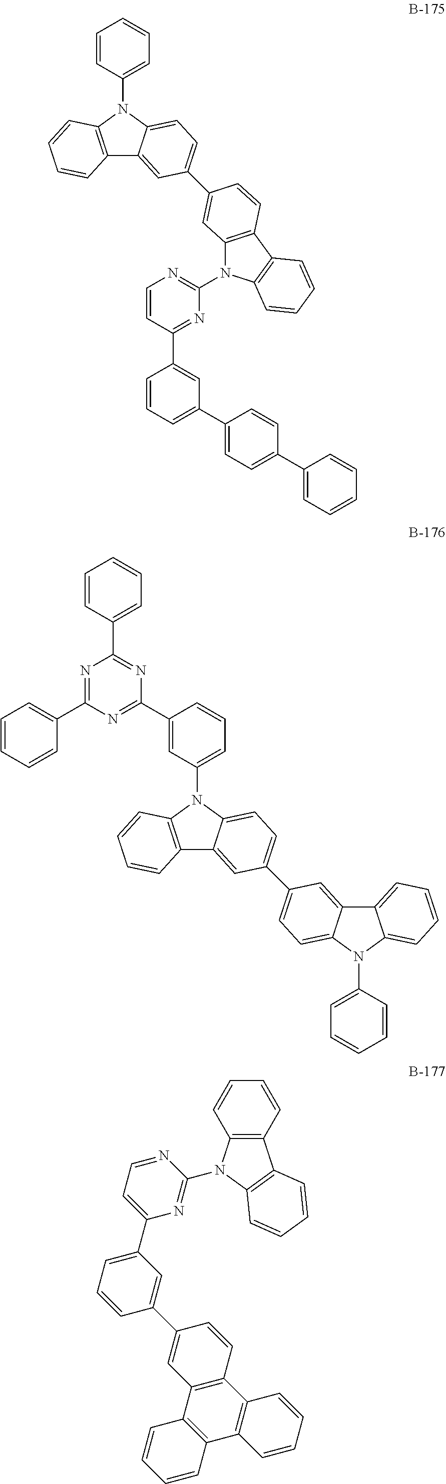 [SMILES: c1ccc(-c2ccc(-c3cccc(-c4ccnc(-n5c6ccccc6c6ccc(-c7ccc8c(c7)c7ccccc7n8-c7ccccc7)cc65)n4)c3)cc2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1cc(-c2ccc3c4ccccc4c4ccccc4c3c2)cc(-c2ccnc(-n3c4ccccc4c4ccccc43)n2)c1]

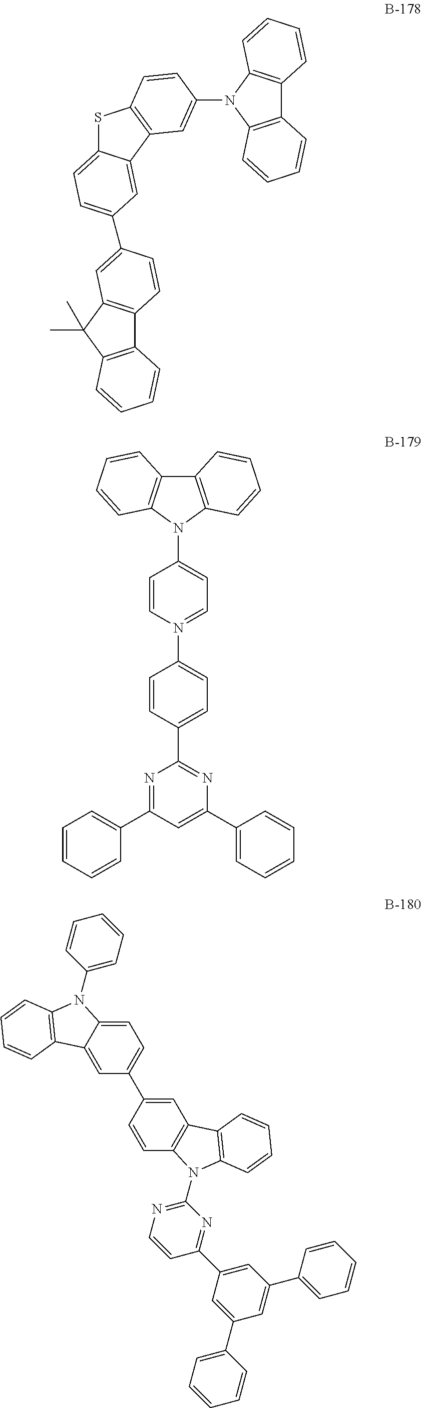

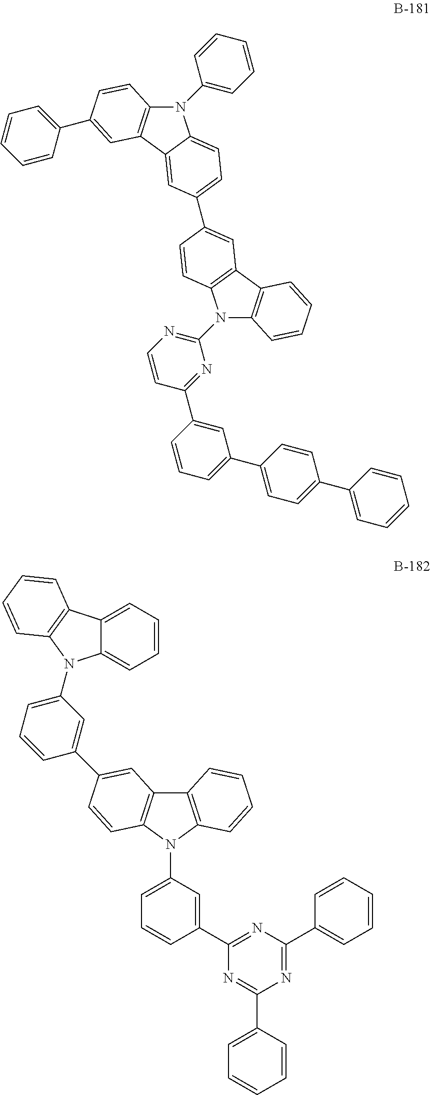 [SMILES: c1ccc(-c2ccc(-c3cccc(-c4ccnc(-n5c6ccccc6c6cc(-c7ccc8c(c7)c7cc(-c9ccccc9)ccc7n8-c7ccccc7)ccc65)n4)c3)cc2)cc1 ; c1ccc(-c2nc(-c3ccccc3)nc(-c3cccc(-n4c5ccccc5c5cc(-c6cccc(-n7c8ccccc8c8ccccc87)c6)ccc54)c3)n2)cc1]

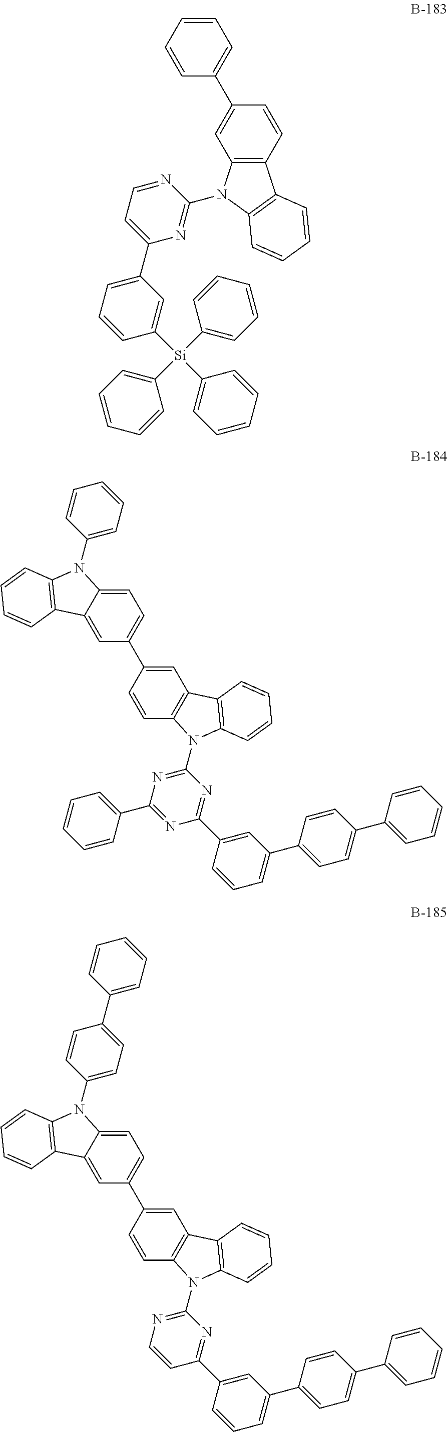 [SMILES: c1ccc(-c2ccc3c4ccccc4n(-c4nccc(-c5cccc([Si](c6ccccc6)(c6ccccc6)c6ccccc6)c5)n4)c3c2)cc1 ; c1ccc(-c2ccc(-c3cccc(-c4nc(-c5ccccc5)nc(-n5c6ccccc6c6cc(-c7ccc8c(c7)c7ccccc7n8-c7ccccc7)ccc65)n4)c3)cc2)cc1 ; c1ccc(-c2ccc(-c3cccc(-c4ccnc(-n5c6ccccc6c6cc(-c7ccc8c(c7)c7ccccc7n8-c7ccc(-c8ccccc8)cc7)ccc65)n4)c3)cc2)cc1]

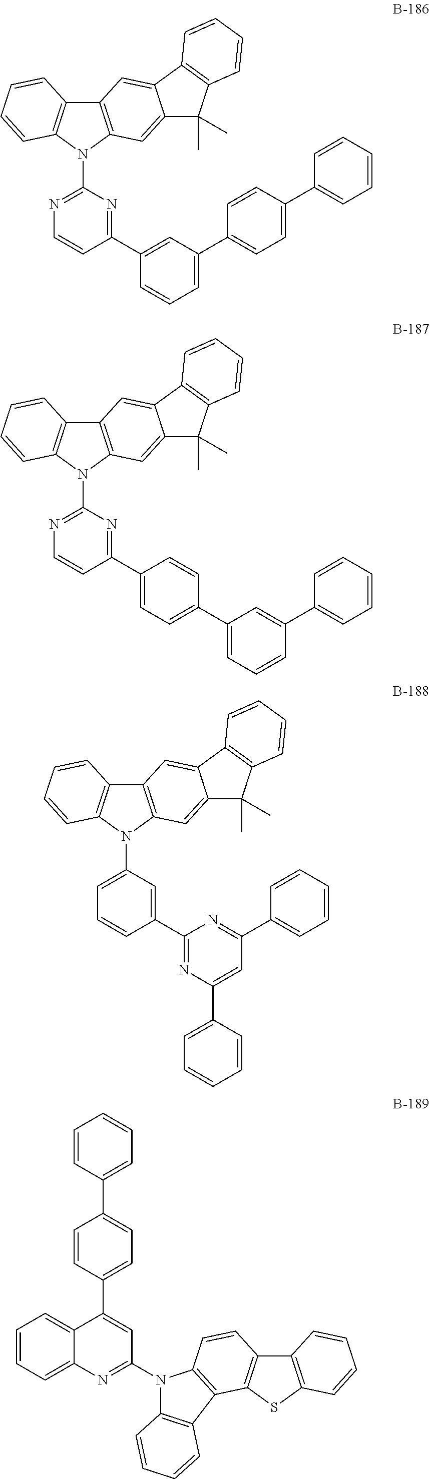

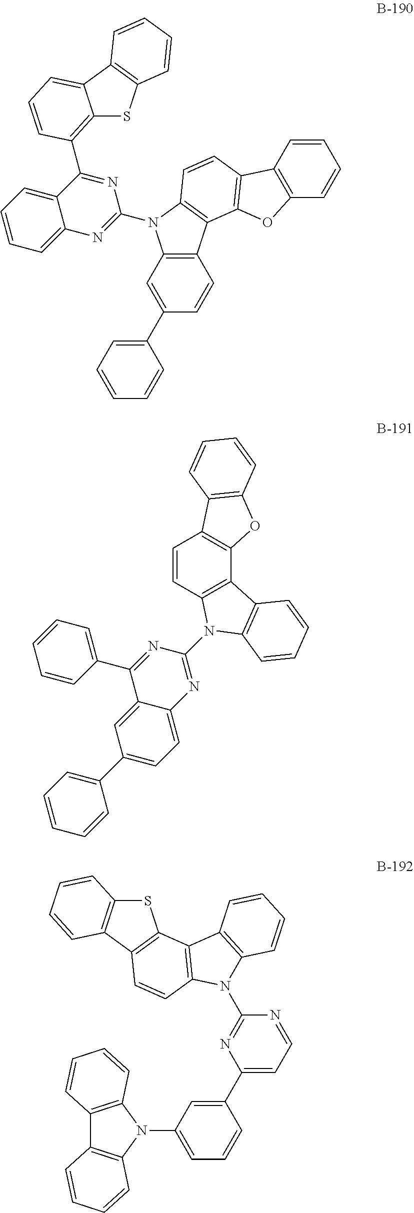

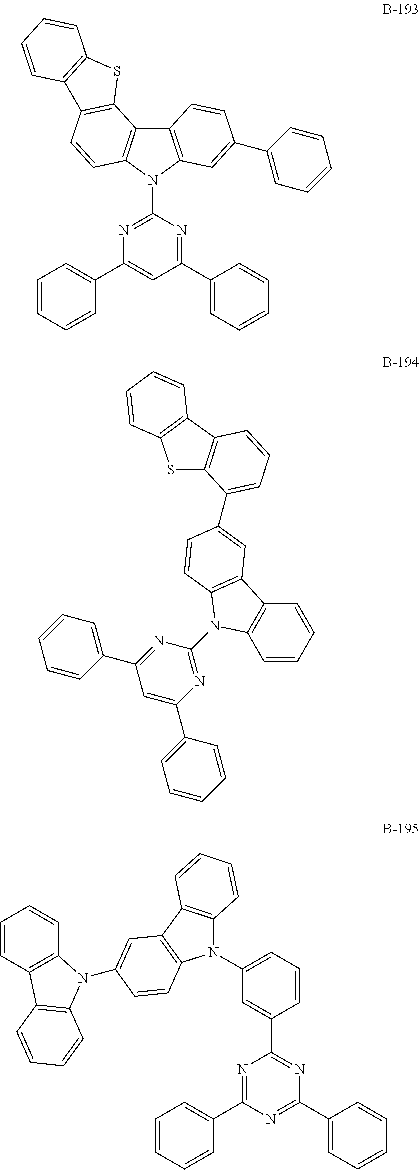 [SMILES: c1ccc(-c2ccc3c4c5sc6ccccc6c5ccc4n(-c4nc(-c5ccccc5)cc(-c5ccccc5)n4)c3c2)cc1 ; c1ccc(-c2cc(-c3ccccc3)nc(-n3c4ccccc4c4cc(-c5cccc6c5sc5ccccc56)ccc43)n2)cc1 ; c1ccc(-c2nc(-c3ccccc3)nc(-c3cccc(-n4c5ccccc5c5cc(-n6c7ccccc7c7ccccc76)ccc54)c3)n2)cc1]

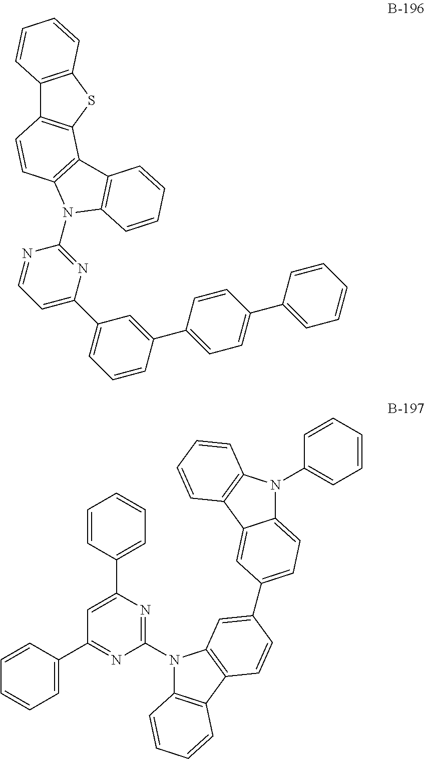 [SMILES: c1ccc(-c2ccc(-c3cccc(-c4ccnc(-n5c6ccccc6c6c7sc8ccccc8c7ccc65)n4)c3)cc2)cc1 ; c1ccc(-c2cc(-c3ccccc3)nc(-n3c4ccccc4c4ccc(-c5ccc6c(c5)c5ccccc5n6-c5ccccc5)cc43)n2)cc1]

[wherein, TPS represents a triphenylsilyl group.]

The dopant comprised in the organic electroluminescence device of the present disclosure is preferably at least one phosphorescent dopant. The phosphorescent dopant material applied to the organic electroluminescence device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescence device of the present disclosure may comprise the compound represented by the following Formula 101, but is not limited thereto:

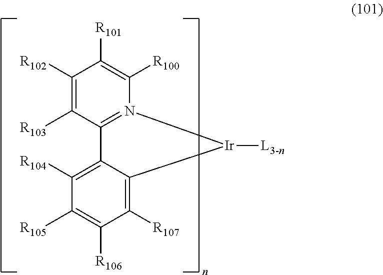

wherein, Formula 101,

L is selected from the following structures 1 and 2:

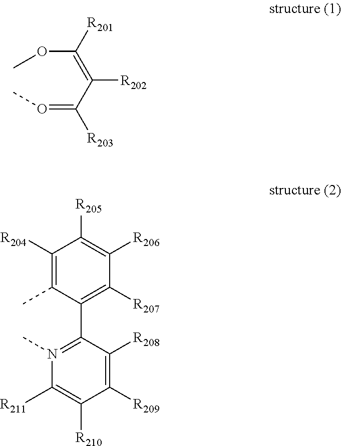

R₁₀₀ to R₁₀₃ each independently represent hydrogen, deuterium, halogen, a halogen-substituted or unsubstituted (C1-C30)alkyl, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or R₁₀₀ to R₁₀₃ may be linked to adjacent substituents to form a substituted or unsubstituted fused ring along with pyridine, e.g., a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;

R₁₀₄ to R₁₀₇ each independently represent hydrogen, deuterium, halogen, a halogen-substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or R₁₀₄ to R₁₀₇ may be linked to adjacent substituents to form a substituted or unsubstituted fused ring along with benzene, e.g., a substituted or unsubstituted naphthyl, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;

R₂₀₁ to R₂₁₁ each independently represent hydrogen, deuterium, halogen, a halogen-substituted or unsubstituted (C1-C30)alkyl, a substituted or unsubstituted (C3-C30)cycloalkyl, a substituted or unsubstituted (C6-C30)aryl; or R₂₀₁ to R₂₁₁ may be linked to adjacent substituents to form a substituted or unsubstituted fused ring;

n represents an integer of 1 to 3.

The specific examples of the dopant compound include the following, but are not limited thereto.

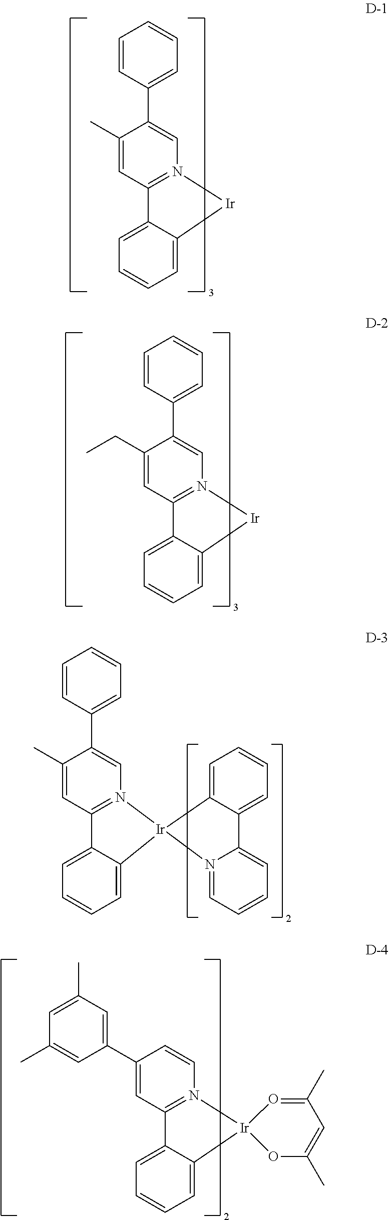

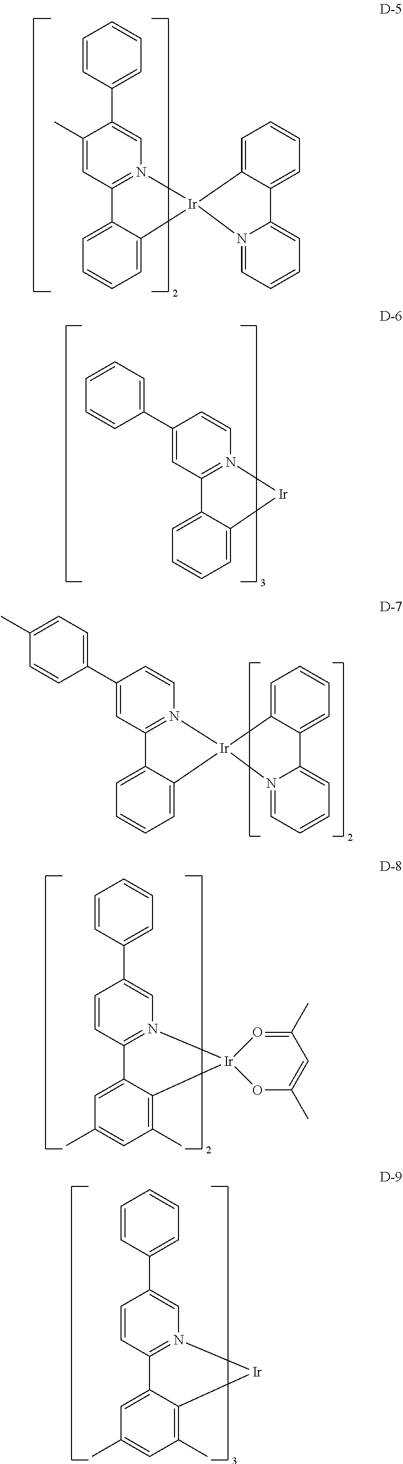

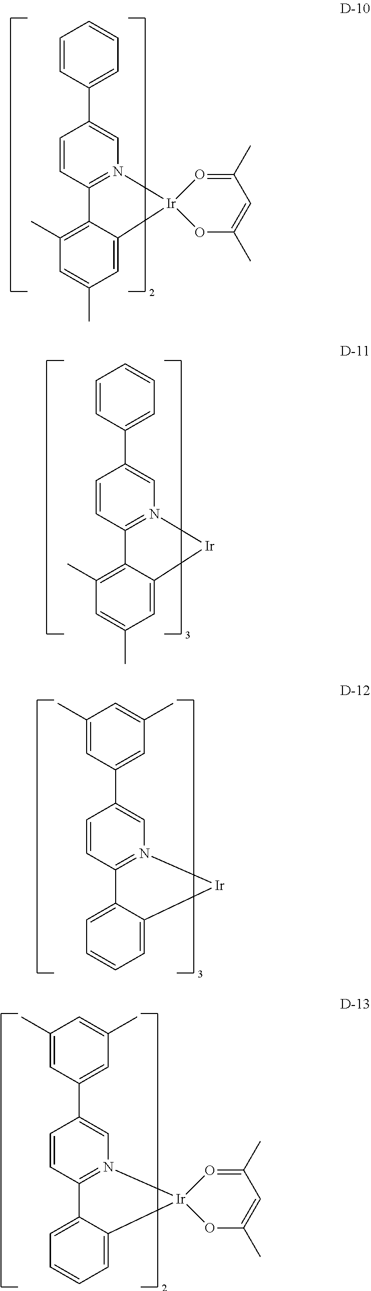

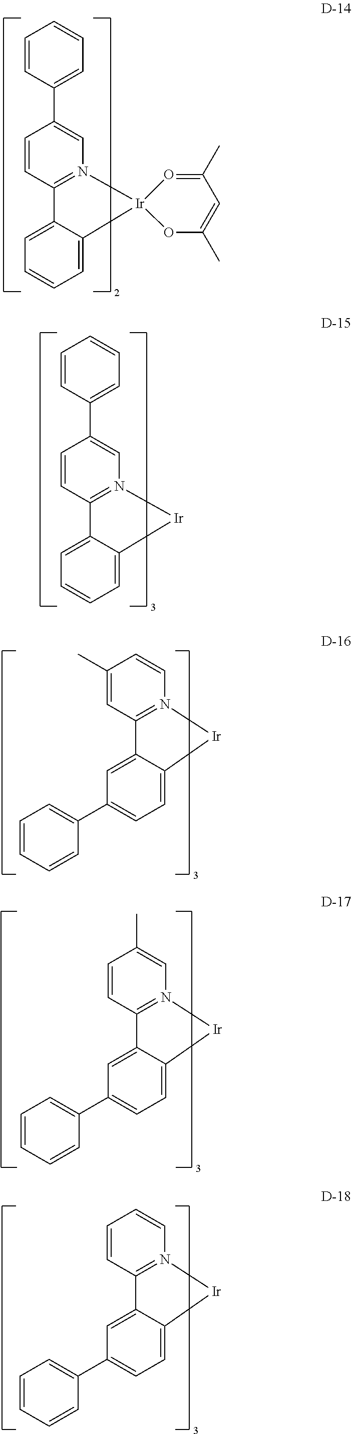

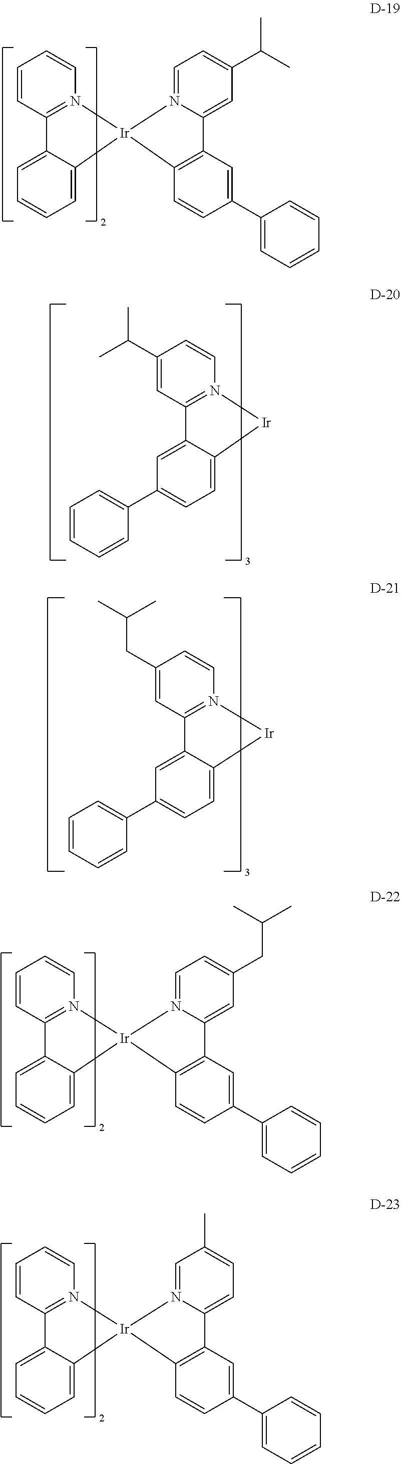

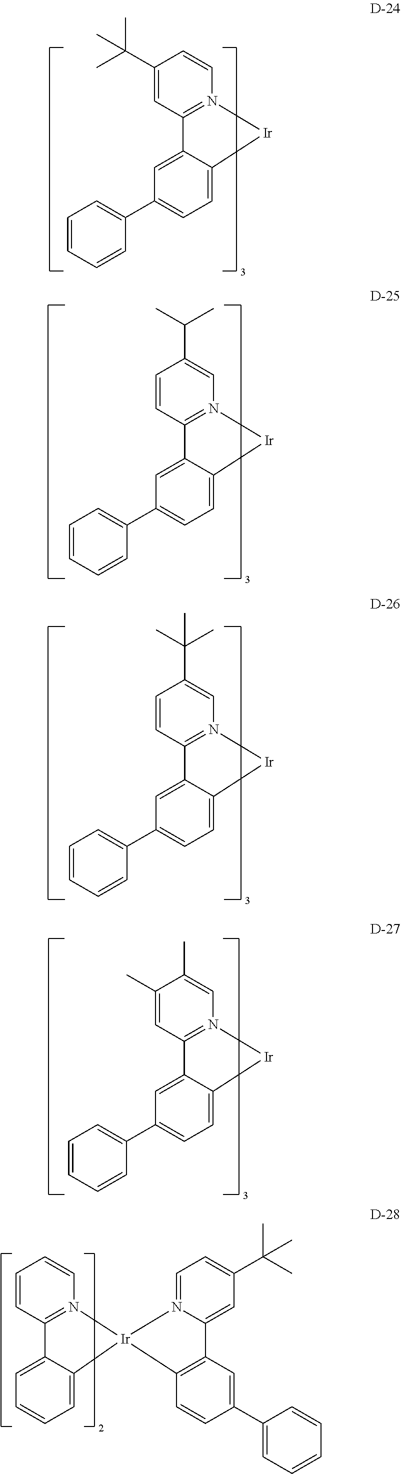

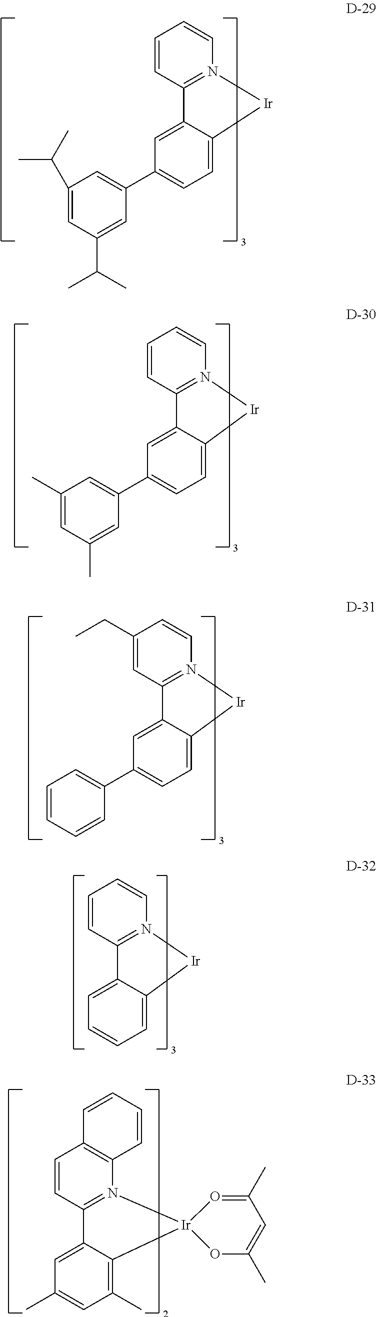

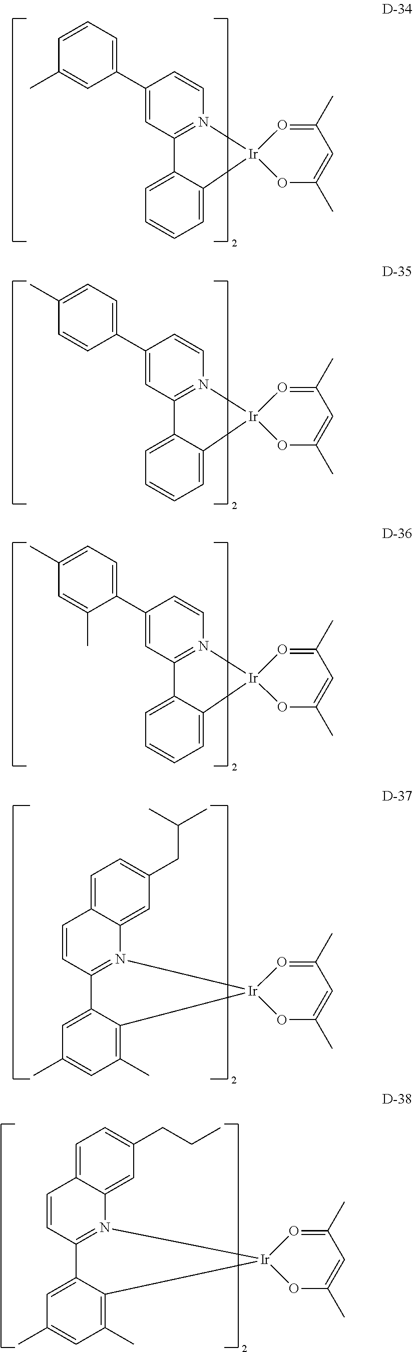

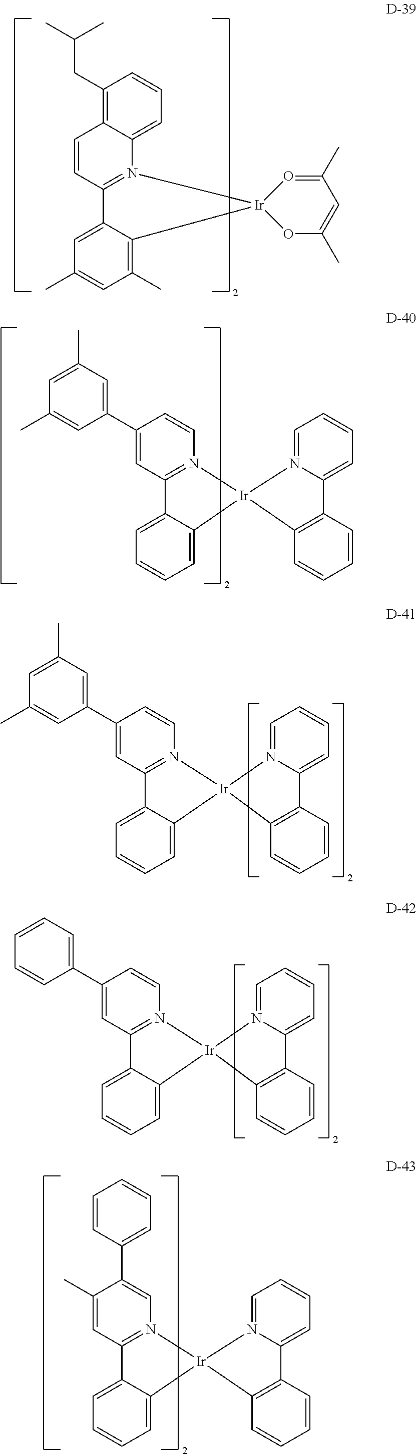

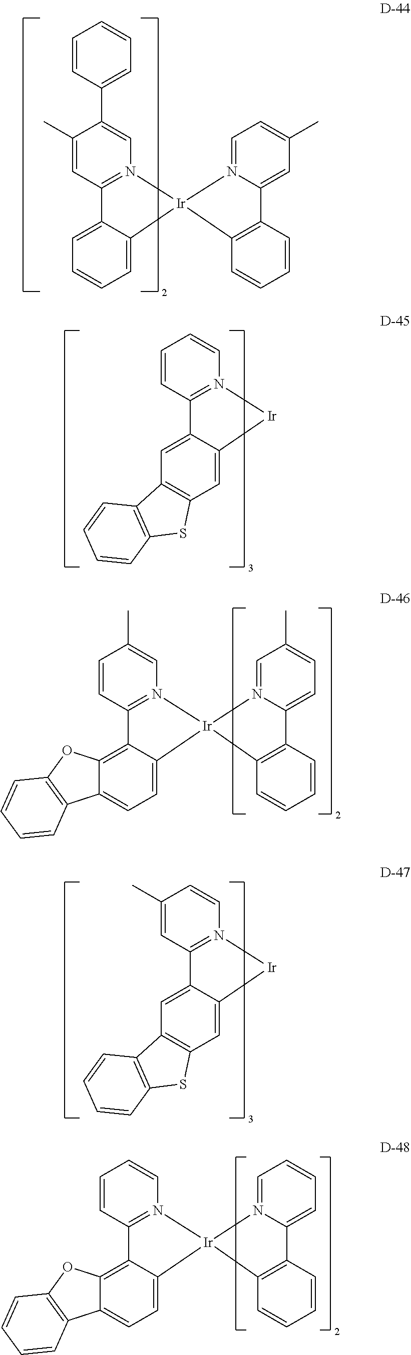

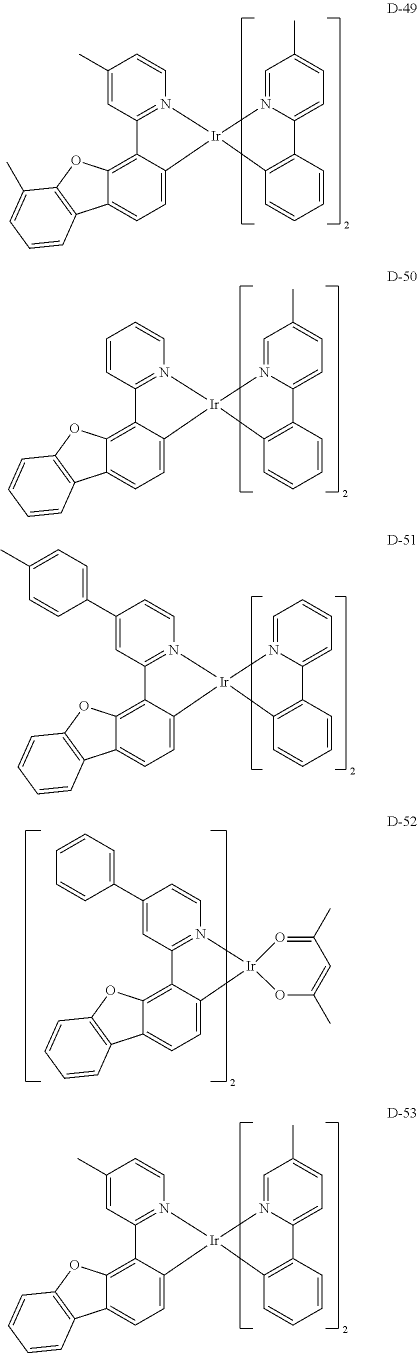

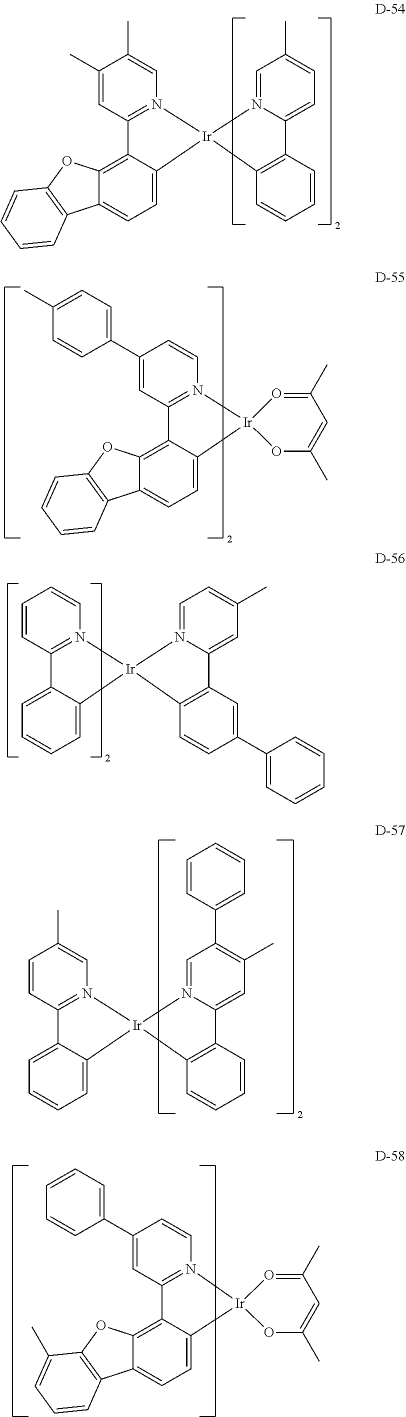

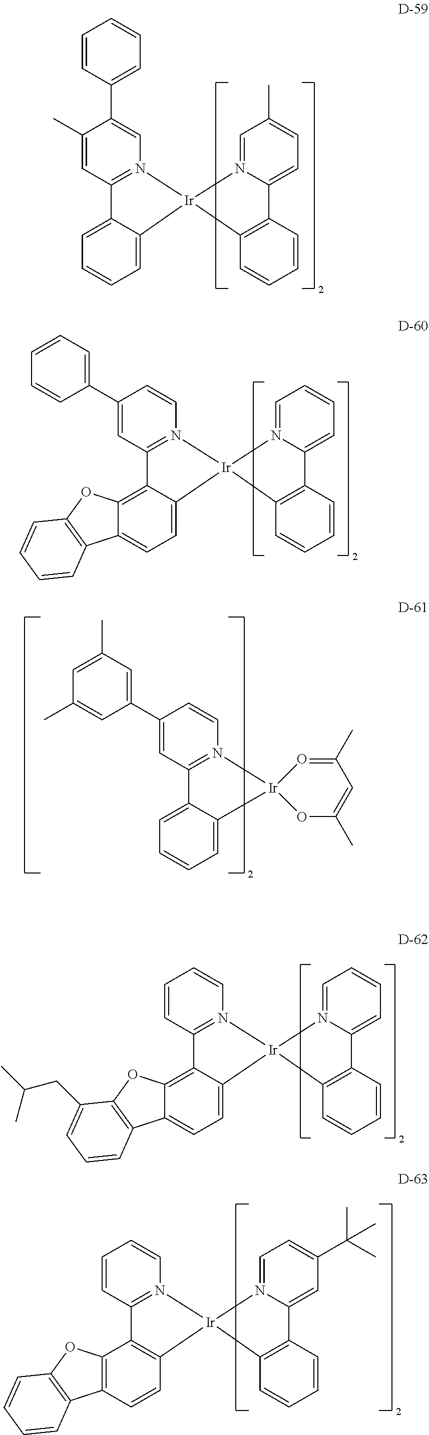

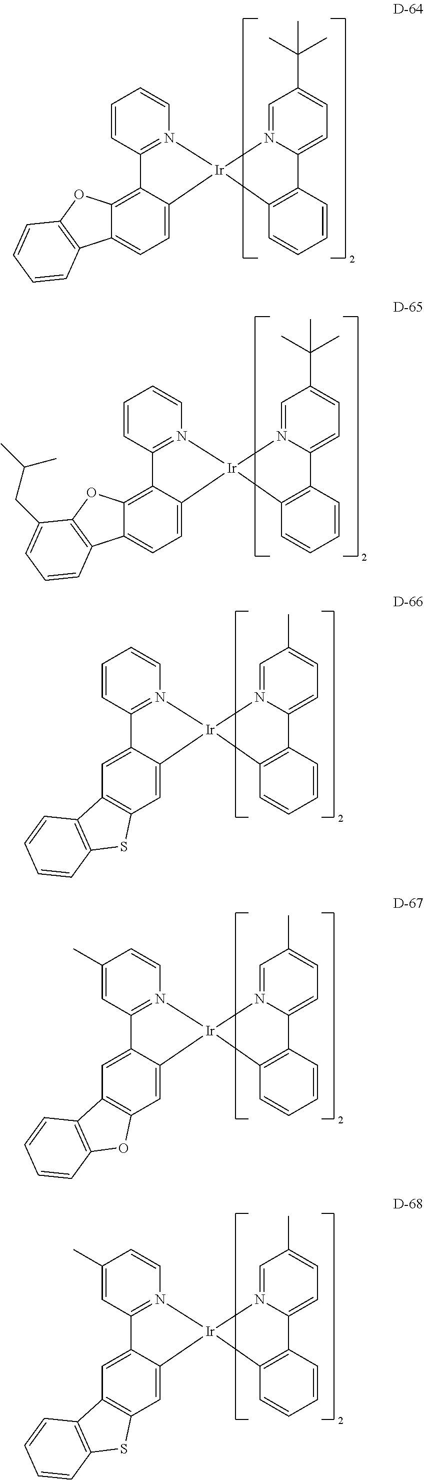

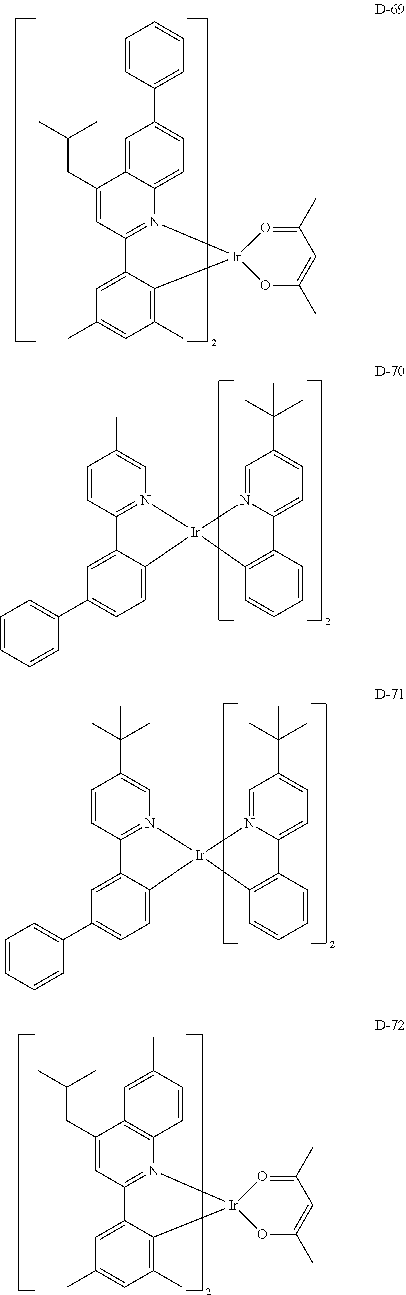

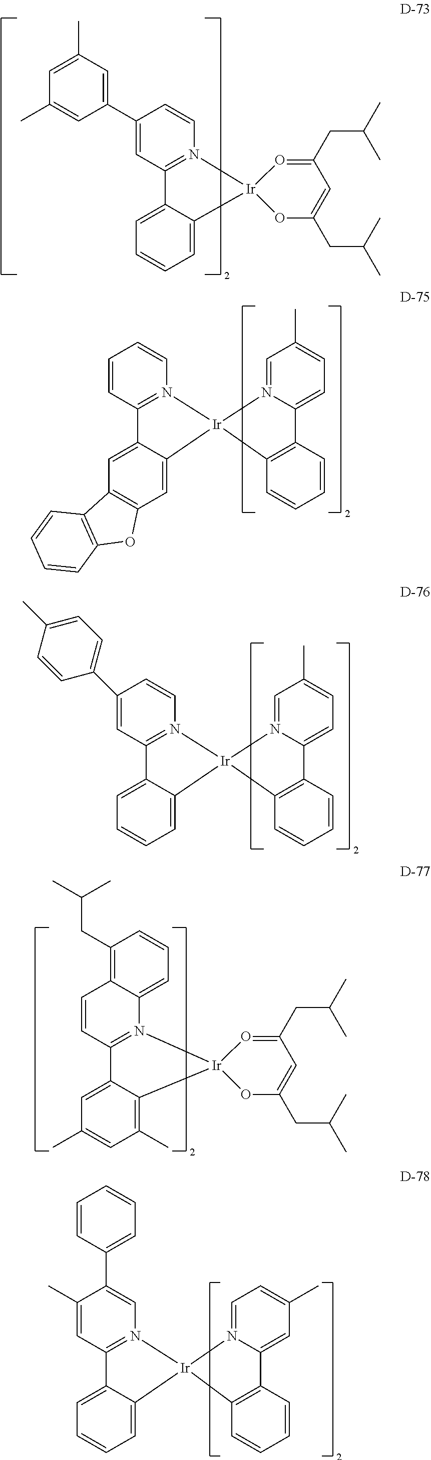

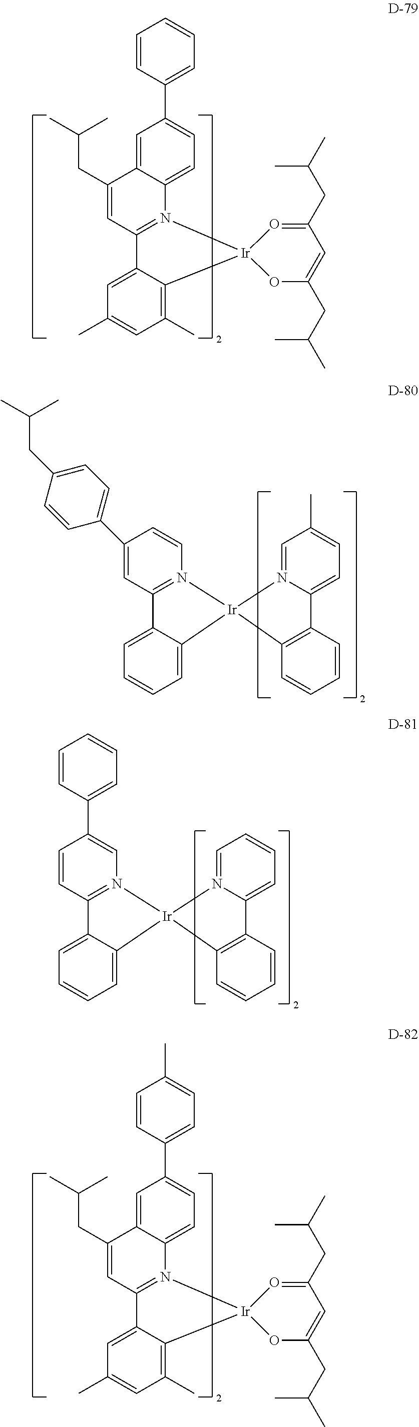

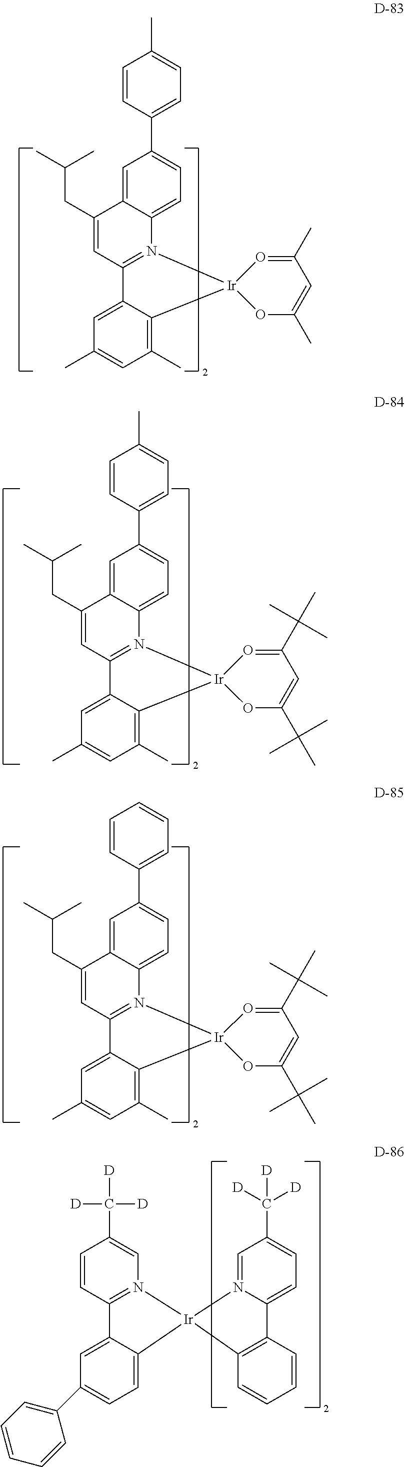

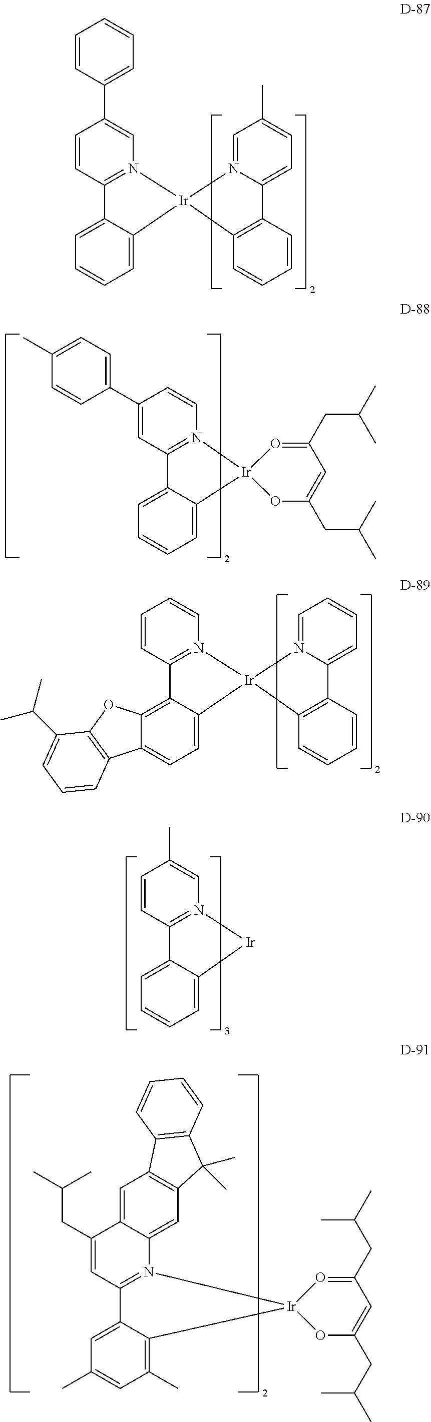

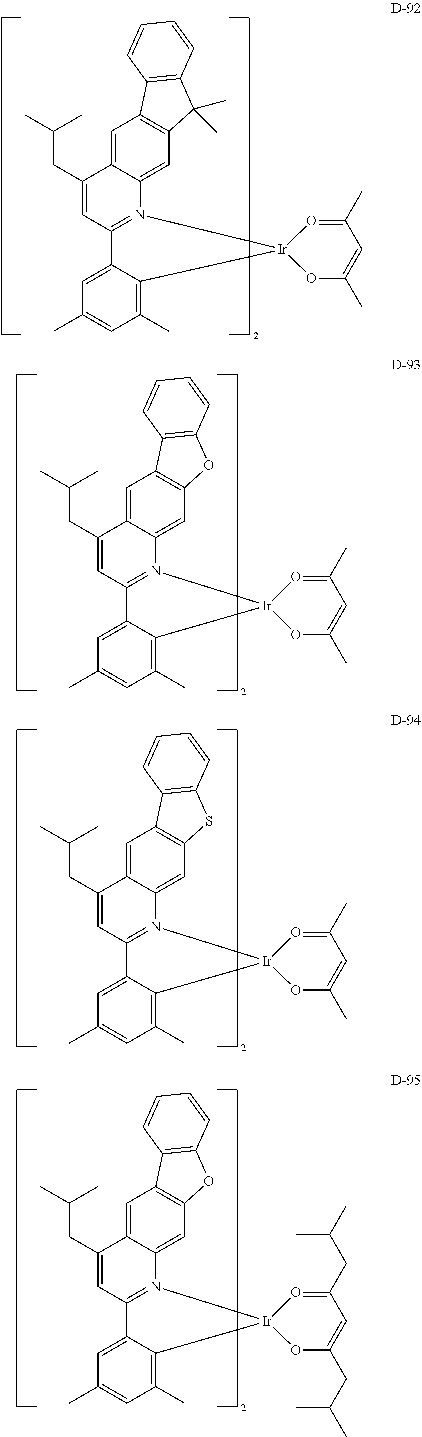

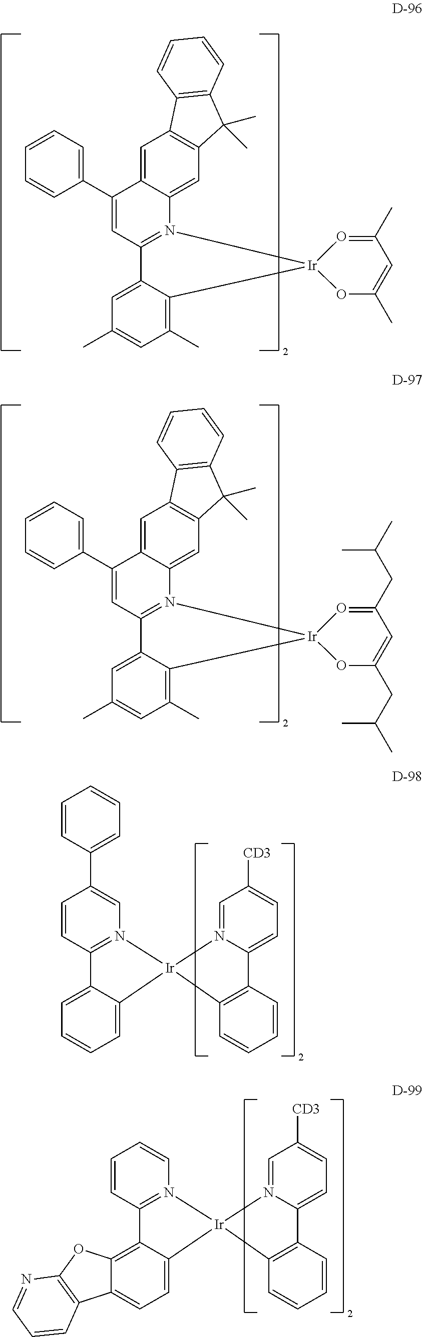

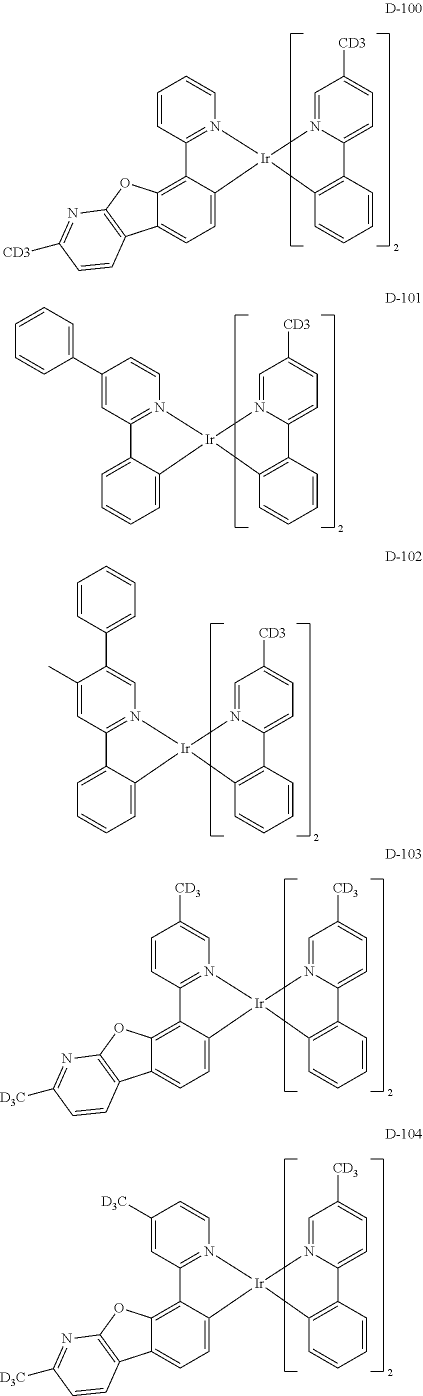

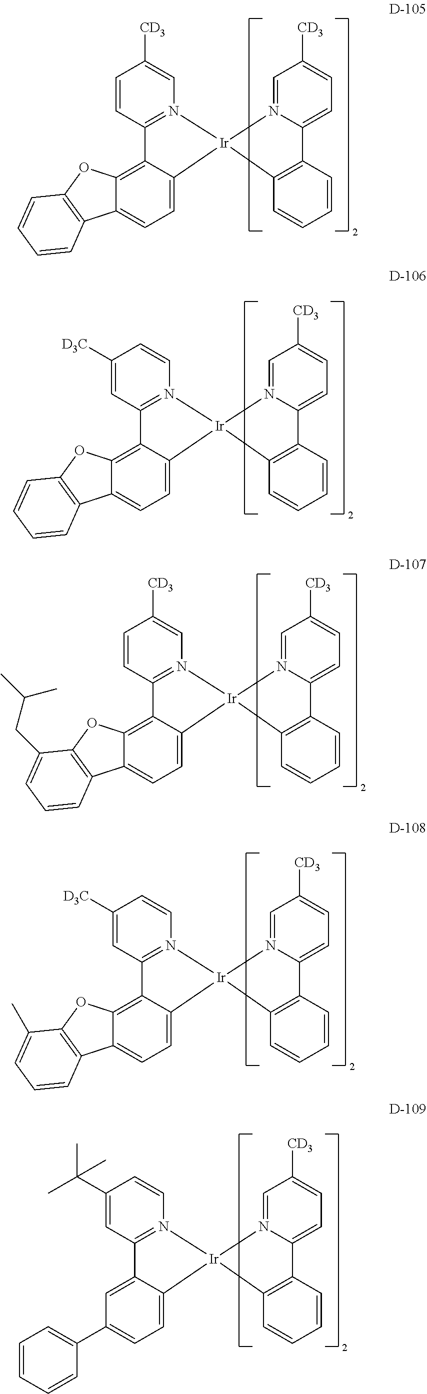

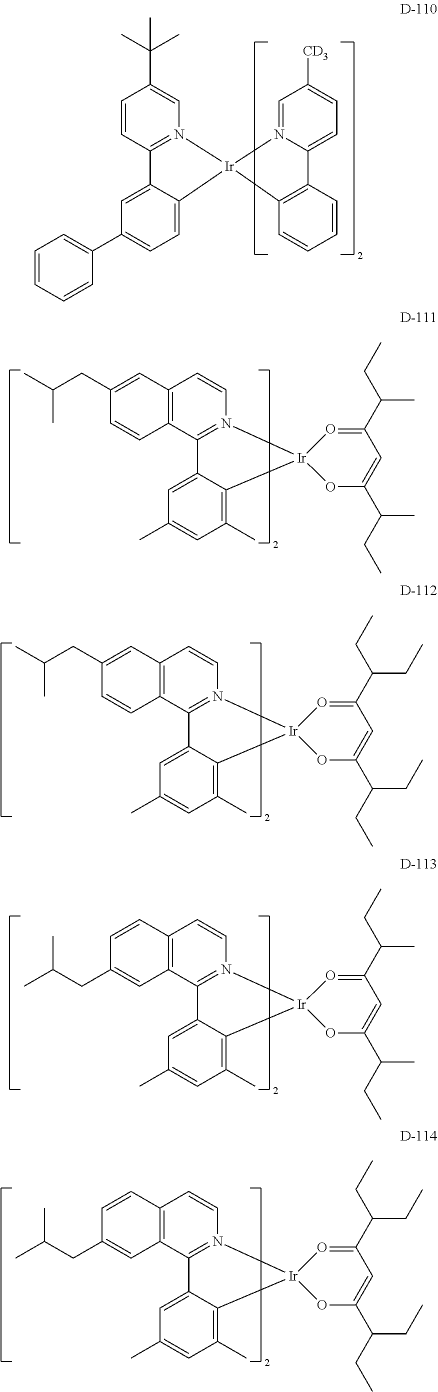

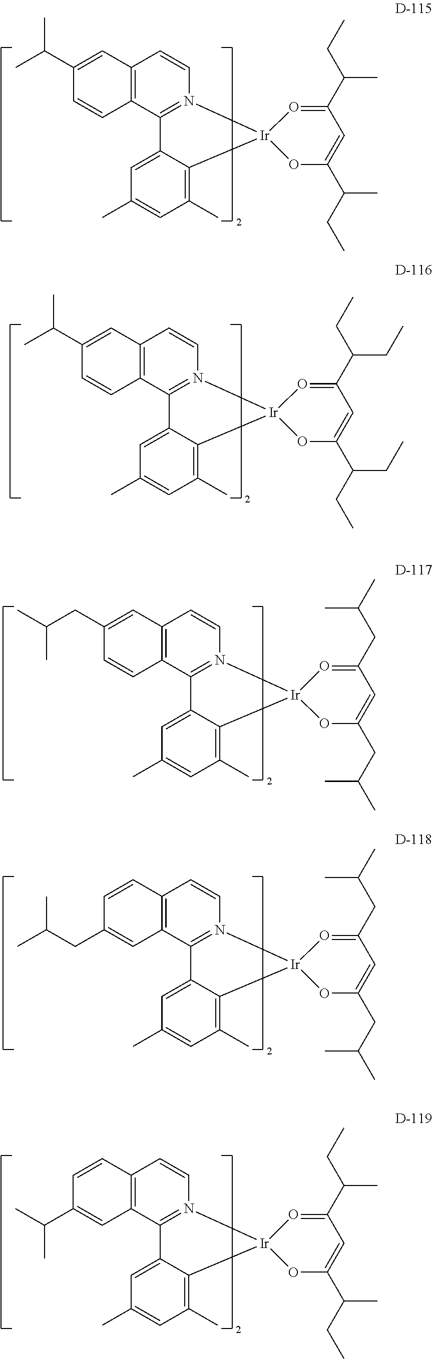

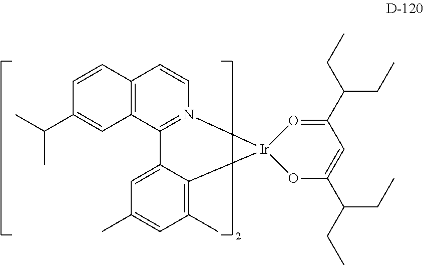

The organic electroluminescence device of the present disclosure may further comprise at least one compound selected from the group consisting of an arylamine compound and a styrylarylamine compound in the intermediate layer.

Also, in the organic electroluminescence device of the present disclosure, the intermediate layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising such a metal.

In the organic electroluminescence device of the present disclosure, preferably, at least one layer selected from a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescence medium layer, and a metal halide layer or a metal oxide layer is preferably placed on a cathode surface of an electroluminescence medium layer. The operation stability for the organic electroluminescence device may be obtained by the surface layer. Preferably, the chalcogenide includes SiO_{X}(1≤X≤2), AlO_{X}(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

The first electrode may be an anode, and a hole transporting band may be disposed between the anode and the light-emitting layer and may include a hole transport layer. In addition to the hole transport layer, a hole injection layer, an electron blocking layer, or a combination of a hole injection layer and an electron blocking layer may be used. The hole injection layer may be formed of a plurality of layers for the purpose of lowering the hole injection barrier (or hole injection voltage) from the anode to the hole transport layer or the electron blocking layer, and each layer may use two compounds at the same time. The electron blocking layer may also be used as a plurality of layers.

The second electrode may be a cathode, and a layer selected from an electron buffer layer, a hole blocking layer, an electron transport layer, or an electron injection layer or a combination thereof may be used between the light-emitting layer and the cathode. The electron buffer layer may be formed of a plurality of layers for the purpose of controlling electron injection and improving interfacial characteristics between the light-emitting layer and the electron injection layer, and each layer may use two compounds at the same time. A plurality of layers may also be used as the hole blocking layer or the electron transporting layer, and a plurality of compounds may be used in each layer.

In addition, in the organic electroluminescence device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescence medium. Furthermore, the hole transport compound is oxidized to a cation, and thus it becomes easier to inject and transport holes from the mixed region to the electroluminescence medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescence device having two or more light-emitting layers and emitting white light.

In order to form each layer of the organic electroluminescence device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

Also, the organic electroluminescence device of the present disclosure can be used for the manufacture of a display device or a lighting device.

Hereinafter, the method of manufacturing a device including the host compound and the hole transport material of the present disclosure and the luminescent characteristics thereof will be described in order to understand the present disclosure in detail.

[Device Example 1] Producing an OLED Device Comprising the Combination of a Hole Transport Material and a Host Compound in Accordance with the Present Disclosure

An OLED device including a combination of a hole transport material and a host compound of the present disclosure was prepared. A transparent electrode indium tin oxide (ITO) thin film (10 Ω/sq) on a glass substrate for an OLED device (GEOMATEC CO., LTD., Japan) was subjected to an ultrasonic washing with acetone, ethanol, and distilled water, sequentially, and then was stored in isopropanol. The ITO substrate was then mounted on a substrate holder of a vacuum vapor deposition apparatus. HI-1 was introduced into a cell of the vacuum vapor deposition apparatus, and then the pressure in the chamber of the apparatus was controlled to 10⁻⁶ torr by air exhaustion. Thereafter, an electric current was applied to the cell to evaporate the above-introduced material, thereby forming a first hole injection layer having a thickness of 90 nm on the ITO substrate. Next, HI-2 was introduced into another cell of the vacuum vapor deposition apparatus, and was evaporated by applying an electric current to the cell, thereby forming a second hole injection layer having a thickness of 5 nm on the first hole injection layer. HT-1 was then introduced into another cell of the vacuum vapor deposition apparatus, and was evaporated by applying an electric current to the cell, thereby forming a first hole transport layer having a thickness of 10 nm on the second hole injection layer. C-10 was then introduced into another cell of the vacuum vapor deposition apparatus, and was evaporated by applying an electric current to the cell, thereby forming a second hole transport layer having a thickness of 60 nm on the first hole transport layer. After forming the hole injection layer and the hole transport layer, a light-emitting layer was formed thereon as follows: H-17 was introduced into one cell of the vacuum vapor depositing apparatus as a host, and D-39 was introduced into another cell as a dopant. The dopant was deposited in a doping amount of 2 wt % based on the total amount of the host and dopant by evaporating the two materials at different rates to form a light-emitting layer having a thickness of 40 nm on the second hole transport layer. ET-1 and EI-1 were then introduced into the other two cells, and were respectively evaporated at a rate of 1:1 to form an electron transport layer having a thickness of 35 nm on the light-emitting layer. After depositing EI-1 as an electron injection layer having a thickness of 2 nm on the electron transport layer, an Al cathode having a thickness of 80 nm was deposited on the electron injection layer with another vacuum vapor deposition apparatus. Thus, an OLED device was produced.

As a result, the minimum time taken to be reduced from 100% to 97% of the luminance at 5,000 nit was 159 hours.

[Device Example 2] Producing an OLED Device Comprising the Combination of a Hole Transport Material and a Host Compound in Accordance with the Present Disclosure

An OLED device was produced in the same manner as in Device Example 1, except for using C-7 as a second hole transport material.

As a result, the minimum time taken to be reduced from 100% to 97% of the luminance at 5,000 nit was 258 hours.

[Device Example 3] Producing an OLED Device Comprising the Combination of a Hole Transport Material and a Host Compound in Accordance with the Present Disclosure

An OLED device was produced in the same manner as in Device Example 1, except for using C-58 as a second hole transport material.

As a result, the minimum time taken to be reduced from 100% to 97% of the luminance at 5,000 nit was 221 hours.

[Device Comparative Example 1] Producing an OLED Device not Comprising the Combination of a Hole Transport Material and a Host Compound in Accordance with the Present Disclosure

An OLED device was produced in the same manner as in Device Example 1, except for using C-10 as a second hole transport material and compound C as a host.

As a result, the minimum time taken to be reduced from 100% to 97% of the luminance at 5,000 nit was 1.8 hours.

[Device Comparative Example 2] Producing an OLED Device not Comprising the Combination of a Hole Transport Material and a Host Compound in Accordance with the Present Disclosure

An OLED device was produced in the same manner as in Device Example 1, except for using C-10 as a second hole transport material and compound D as a host.

As a result, the minimum time taken to be reduced from 100% to 97% of the luminance at 5,000 nit was 21.4 hours.

[Device Comparative Example 3] Producing an OLED Device not Comprising the Combination of a Hole Transport Material and a Host Compound in Accordance with the Present Disclosure

An OLED device was produced in the same manner as in Device Example 1, except for using compound A as a second hole transport material and H-17 as a host.

As a result, the minimum time taken to be reduced from 100% to 97% of the luminance at 5,000 nit was 16.5 hours.

[Device Comparative Example 4] Producing an OLED Device not Comprising the Combination of a Hole Transport Material and a Host Compound in Accordance with the Present Disclosure

An OLED device was produced in the same manner as in Device Example 1, except for using compound B as a second hole transport material and H-17 as a host.

As a result, the minimum time taken to be reduced from 100% to 97% of the luminance at 5,000 nit was 137 hours.

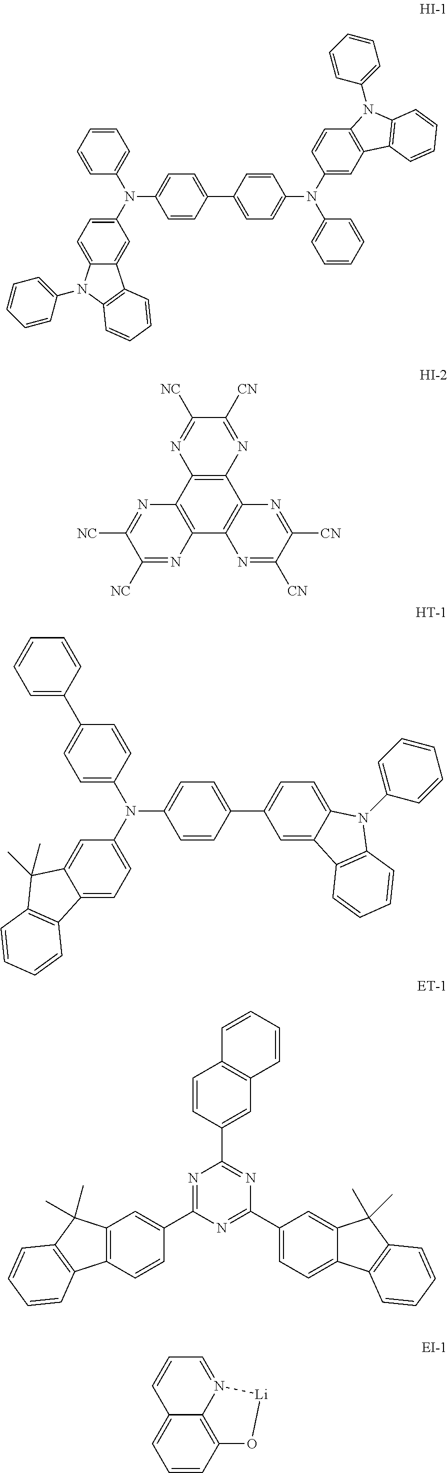

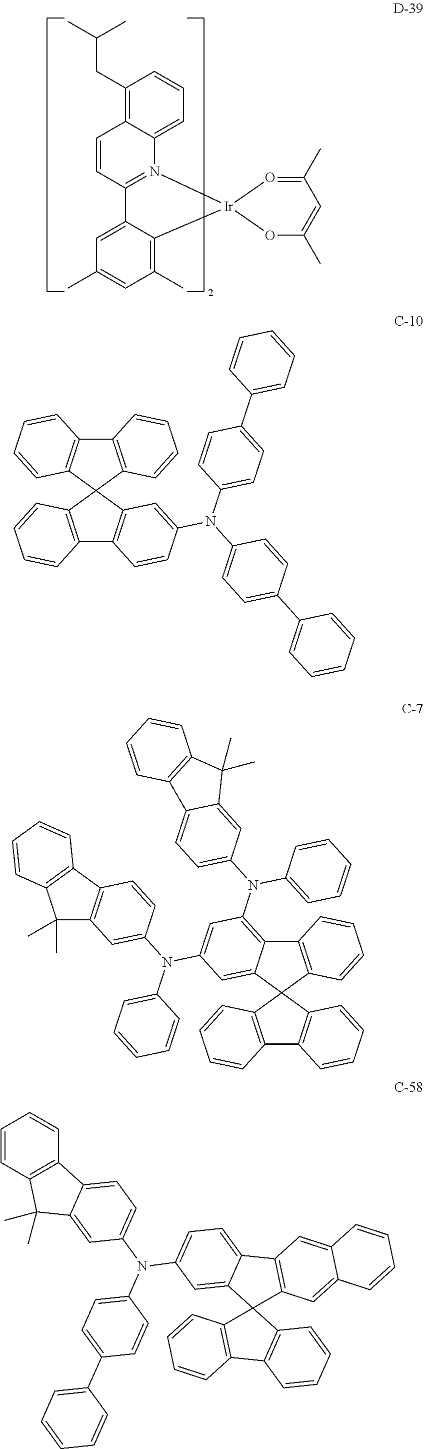

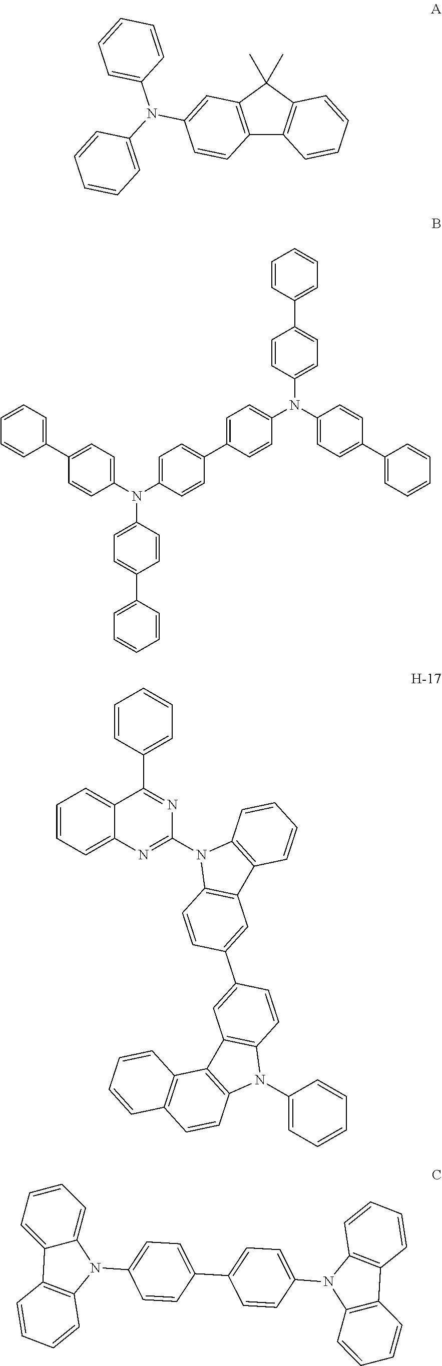

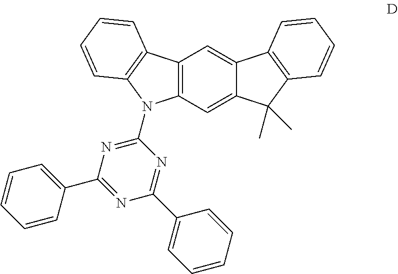 [SMILES: CC1(C)c2ccccc2-c2cc3c4ccccc4n(-c4nc(-c5ccccc5)nc(-c5ccccc5)n4)c3cc21]

The present disclosure has confirmed that the organic electroluminescence device is manufactured by using a combination of a specific hole transport material and a host compound, so that the driving lifetime is much better than that of a conventional organic electroluminescence device.

That is, the HOMO (highest occupied molecular orbital) energy level of the compound comprising spirofluorene used in a hole transport material is formed to be 4.7 to 4.8 eV. The compound in which a benzene ring is fused to one of two carbazole of a biscarbazole structure used as a host material of a light-emitting layer and heteroaryl containing nitrogen is bonded to one of two nitrogen atoms, has a HOMO energy level of 5.0 eV, so that the hole injection ability can be improved due to the relatively low energy barrier, thereby reducing the deterioration phenomenon at the interface between the hole transport layer and the light-emitting layer, and improving the lifespan of the device.

The present disclosure relates to the combination of a benzo-HOMO (highest occupied molecular orbital) site of a cabazole group and a spirofluorene group so that it is possible to smoothly transfer a hole to the light-emitting layer in the organic electroluminescence device. The phenomenon extends the recombination region to generate more excitons and recombine more electron-hole pairs. Thus, the device of the present disclosure can have better lifespan characteristics than the devices containing each of the aforementioned components.

1. An organic electroluminescence device comprising a first electrode; a second electrode facing the first electrode; an intermediate layer between the first electrode and the second electrode; wherein the intermediate layer comprises at least one layer of a hole transporting band and at least one layer of a light-emitting layer; wherein at least one layer of the hole transporting band comprises a compound represented by the following Formula 1; wherein at least one layer of the light-emitting layer comprises at least one dopant compound and at least one host compound; and the at least one host compound comprises a compound represented by the following Formula 2: 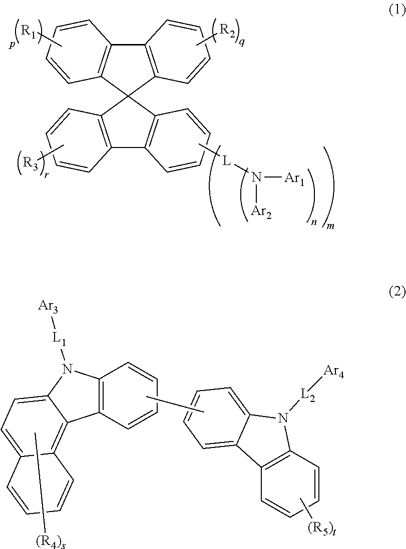
wherein the Formulae 1 and 2,
L, L₁ and L₂ each independently represent a single bond or a substituted or unsubstituted (C6-C30)arylene;
Ar₁ to Ar₄ each independently represent a substituted or unsubstituted (C6-C30)aryl or a substituted or unsubstituted (3- to 30-membered)heteroaryl;
R₁ to R₅ each independently represent hydrogen, deuterium, halogen, cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; or may be linked to an adjacent substituent to form a ring of a substituted or unsubstituted, (C3-C30) mono- or polycyclic, alicyclic or aromatic ring, or the combination thereof, whose carbon atom may be replaced with at least one heteroatom selected from nitrogen, oxygen, and sulfur;
m and n each independently represent an integer of 1 or 2;
p, q, r, and t each independently represent an integer of 1 to 4;
s represents an integer of 1 to 6;
when m, n, p, q, r, s or t represents an integer of 2 or more, each of [L-(NAr₁Ar₂)ₙ], each of (NAr₁Ar₂), each of R₁, each of R₂, each of R₃, each of R₄ or each of R₅ may be the same or different; and
the heteroaryl contains at least one heteroatom selected from B, N, O, S, Si, and P.

2. The organic electroluminescence device according to claim 1, wherein the substituents of the substituted (C1-C30)alkyl, the substituted (C2-C30)alkenyl, the substituted (C2-C30)alkynyl, the substituted (C3-C30)cycloalkyl, the substituted (C6-C30)aryl(ene), the substituted (3- to 30-membered)heteroaryl, the substituted tri(C1-C30)alkylsilyl, the substituted tri(C6-C30)arylsilyl, the substituted di(C1-C30)alkyl(C6-C30)arylsilyl, the substituted (C1-C30)alkyldi(C6-C30)arylsilyl, the substituted mono- or di-(C1-C30)alkylamino, the substituted mono- or di-(C6-C30)arylamino, and the substituted (C3-C30) mono- or polycyclic, alicyclic, aromatic, or combination thereof in L, L₁, L₂, Ar₁ to Ar₄, and R₁ to R₅ each independently represent at least one selected from the group consisting of deuterium, halogen, cyano, carboxyl, nitro, hydroxyl, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, a (C6-C30)aryl-substituted or unsubstituted (5- to 30-membered)heteroaryl, a (5- to 30-membered)heteroaryl-substituted or unsubstituted (C6-C30)aryl, tri(C1-C30)alkylsilyl, tri(C6-C30)arylsilyl, di(C1-C30)alkyl(C6-C30)arylsilyl, (C1-C30)alkyldi(C6-C30)arylsilyl, amino, mono- or di-(C1-C30)alkylamino, a (C1-C30)alkyl-substituted or unsubstituted mono- or di-(C6-C30)arylamino, (C1-C30)alkyl(C6-C30)arylamino, (C1-C30)alkylcarbonyl, (C1-C30)alkoxycarbonyl, (C6-C30)arylcarbonyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl.

3. The organic electroluminescence device according to claim 1, wherein the Formula 1 is represented by any one of the following Formulae 3 to 5: 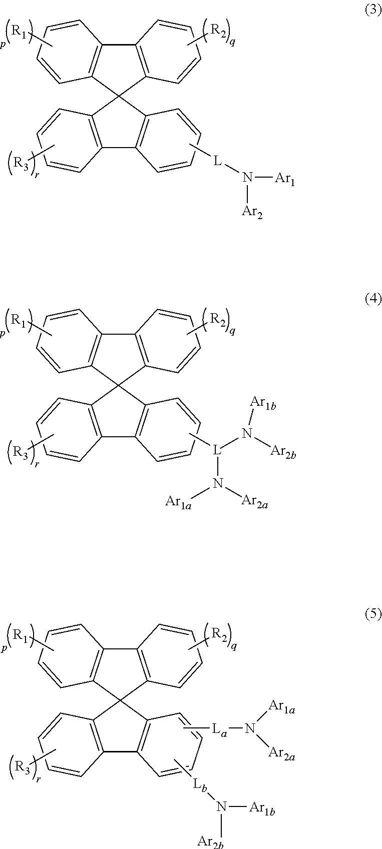
wherein Formulae 3 to 5,
Lₐ and L_{b} are as defined in L;
Ar₁ₐ and Ar_{1b} are as defined in Ar₁;
Ar₂ₐ and Ar_{2b} are as defined in Ar₂; and
L, Ar₁, Ar₂, R₁ to R₃, p, q, and r are as defined in claim 1.

4. The organic electroluminescence device according to claim 3, wherein Ar₁, Ar₂, Ar₁ₐ, Ar₂ₐ, Ar_{1b}, and Ar_{2b} are each independently represented by any one of the following Formulae R-1 to R-9:

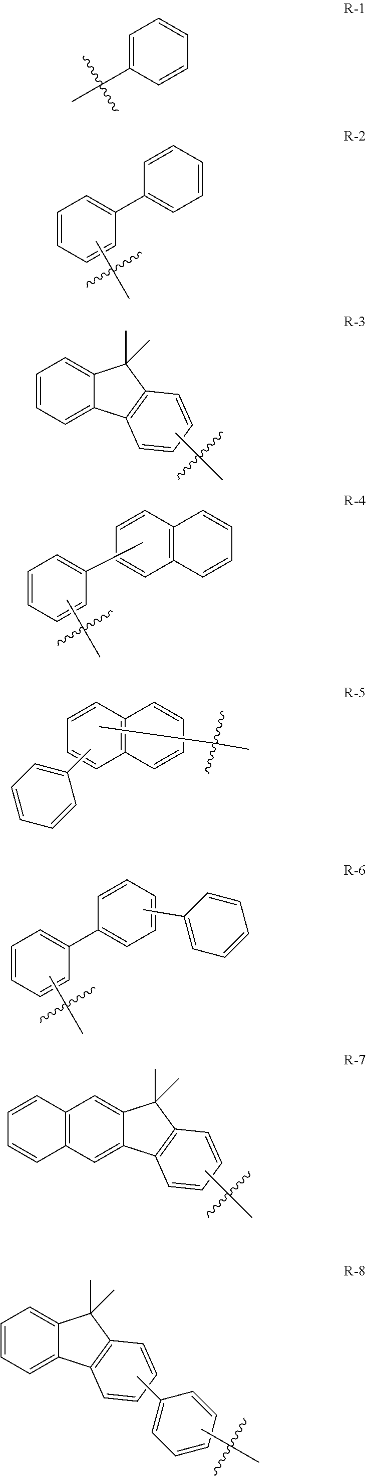

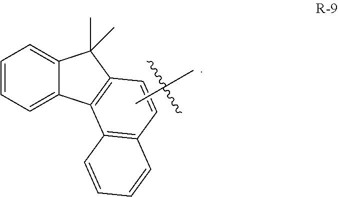

5. The organic electroluminescence device according to claim 1, wherein the Formula 2 is represented by the following Formula 6 or 7: 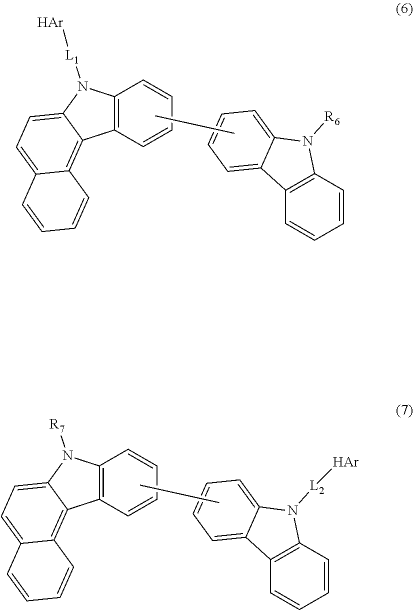
wherein Formulae 6 and 7,
HAr represents a substituted or unsubstituted (5- to 30-membered)heteroaryl containing nitrogen;
L₁ and L₂ each independently represent a single bond or a substituted or unsubstituted (C6-C30)arylene;
R₆ and R₇ each independently represent a substituted or unsubstituted (C6-C30)aryl.

6. The organic electroluminescence device according to claim 1, wherein Formula 2, Ar₃ and Ar₄ each independently represent phenyl, biphenyl, naphthylphenyl, phenylnaphthyl, terphenyl, anthracenyl, phenanthrenyl, di(C1-C6)alkylfluorenyl, quinazolinyl substituted with phenyl, quinazolinyl substituted with di(C1-C6) alkylphenyl, quinazolinyl substituted with naphthylphenyl, quinazolinyl substituted with phenylnaphthyl, quinazolinyl substituted with terphenyl, quinazolinyl substituted with anthracenyl, quinazolinyl substituted with phenanthrenyl, quinazolinyl substituted with biphenyl, quinazolinyl substituted with di(C1-C6)alkylfluorenyl, quinazolinyl substituted with phenylcarbazolyl, quinoxalinyl substituted with phenyl, quinoxalinyl substituted with naphthylphenyl, quinoxalinyl substituted with phenylnaphthyl, quinoxalinyl substituted with terphenyl, quinoxalinyl substituted with anthracenyl, quinoxalinyl substituted with phenanthrenyl, quinoxalinyl substituted with biphenyl, quinoxalinyl substituted with di(C1-C6)alkylfluorenyl, or quinoxalinyl substituted with phenylcarbazolyl.

7. The organic electroluminescence device according to claim 1, wherein the compound represented by Formula 1 is selected from the group consisting of:

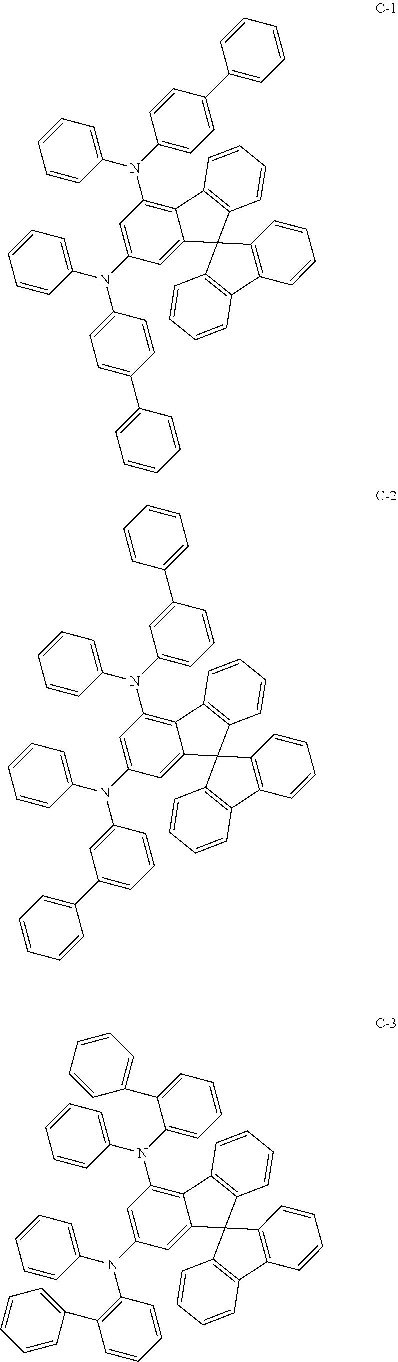

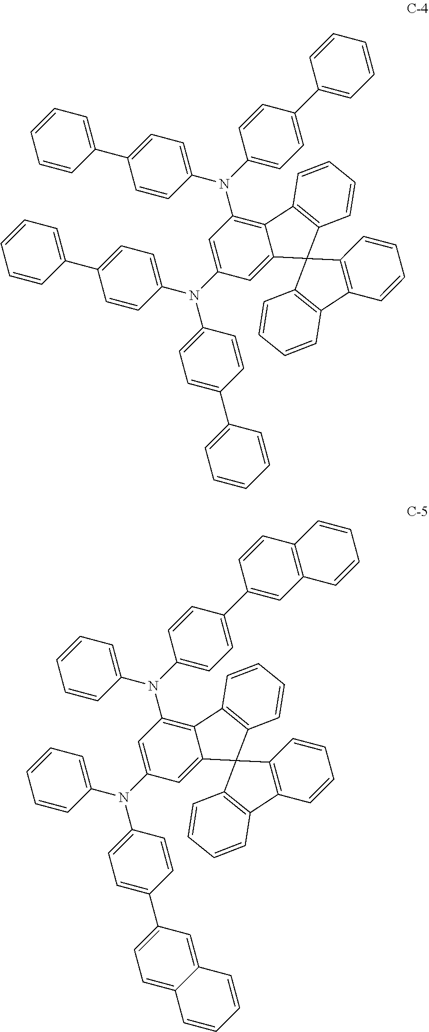 [SMILES: c1ccc(-c2ccc(N(c3ccc(-c4ccccc4)cc3)c3cc(N(c4ccc(-c5ccccc5)cc4)c4ccc(-c5ccccc5)cc4)c4c(c3)C3(c5ccccc5-c5ccccc53)c3ccccc3-4)cc2)cc1 ; c1ccc(N(c2ccc(-c3ccc4ccccc4c3)cc2)c2cc(N(c3ccccc3)c3ccc(-c4ccc5ccccc5c4)cc3)c3c(c2)C2(c4ccccc4-c4ccccc42)c2ccccc2-3)cc1]

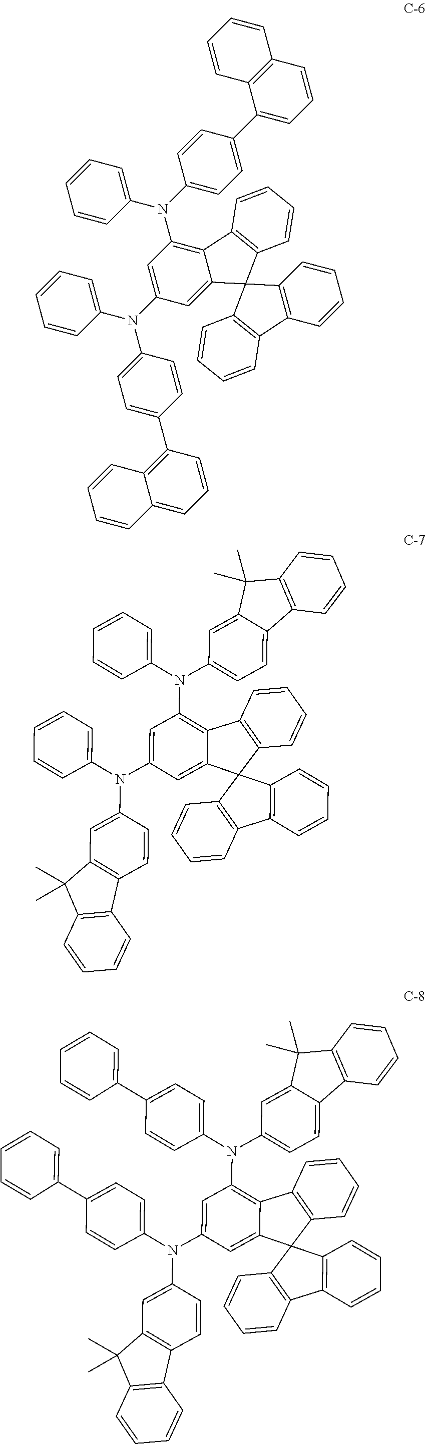 [SMILES: c1ccc(N(c2ccc(-c3cccc4ccccc34)cc2)c2cc(N(c3ccccc3)c3ccc(-c4cccc5ccccc45)cc3)c3c(c2)C2(c4ccccc4-c4ccccc42)c2ccccc2-3)cc1 ; CC1(C)c2ccccc2-c2ccc(N(c3ccccc3)c3cc(N(c4ccccc4)c4ccc5c(c4)C(C)(C)c4ccccc4-5)c4c(c3)C3(c5ccccc5-c5ccccc53)c3ccccc3-4)cc21 ; CC1(C)c2ccccc2-c2ccc(N(c3ccc(-c4ccccc4)cc3)c3cc(N(c4ccc(-c5ccccc5)cc4)c4ccc5c(c4)C(C)(C)c4ccccc4-5)c4c(c3)C3(c5ccccc5-c5ccccc53)c3ccccc3-4)cc21]

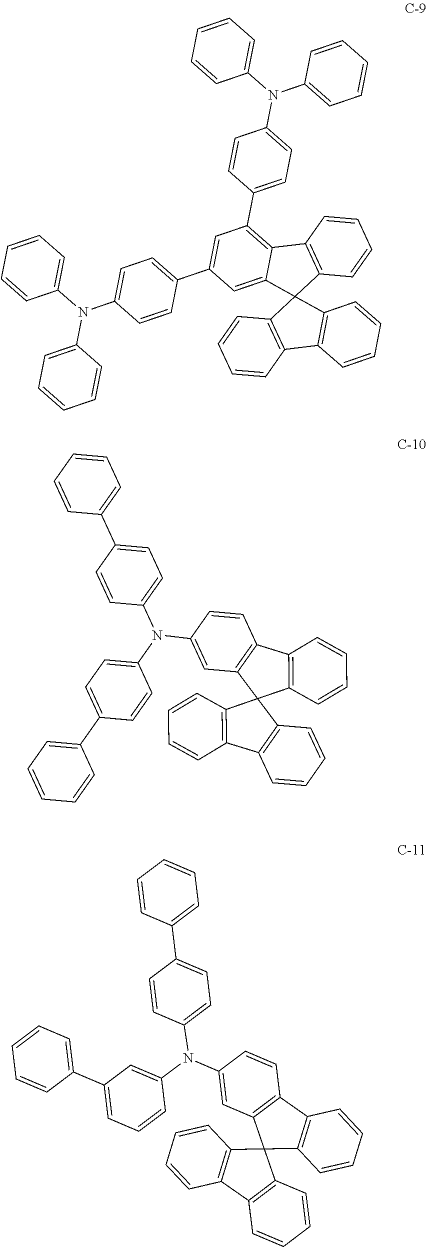

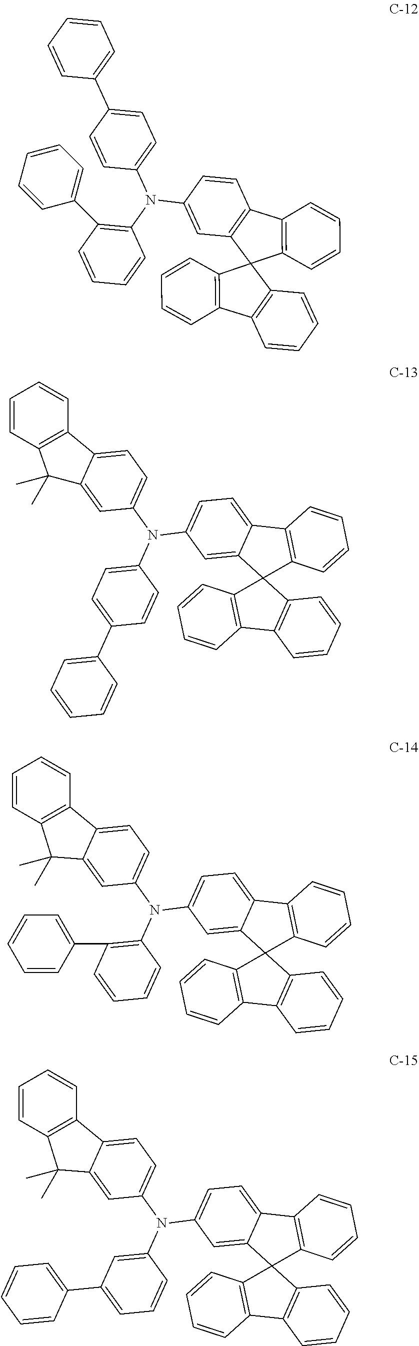 [SMILES: c1ccc(-c2ccc(N(c3ccc4c(c3)C3(c5ccccc5-c5ccccc53)c3ccccc3-4)c3ccccc3-c3ccccc3)cc2)cc1 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; CC1(C)c2ccccc2-c2ccc(N(c3cccc(-c4ccccc4)c3)c3ccc4c(c3)C3(c5ccccc5-c5ccccc53)c3ccccc3-4)cc21]

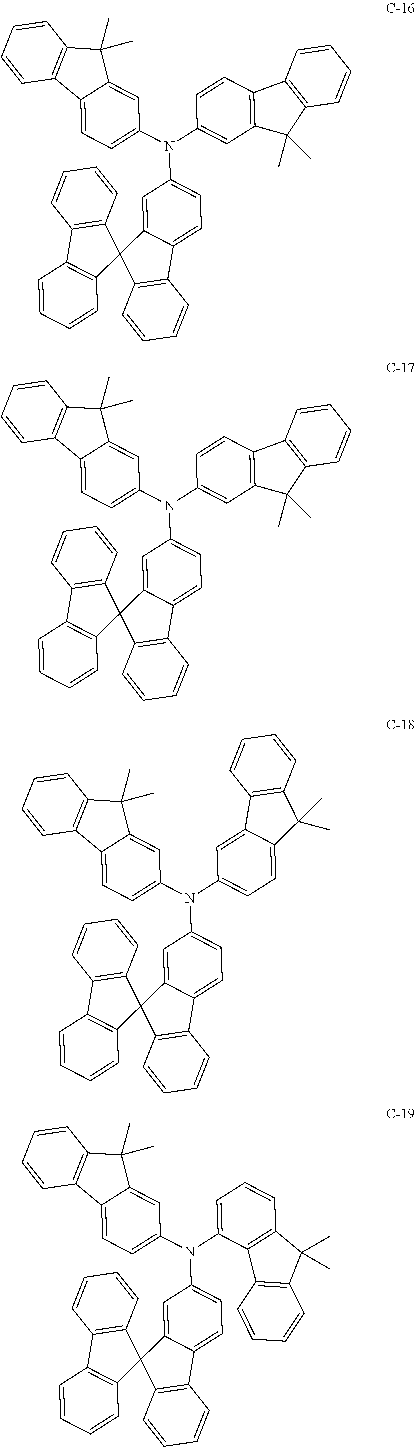

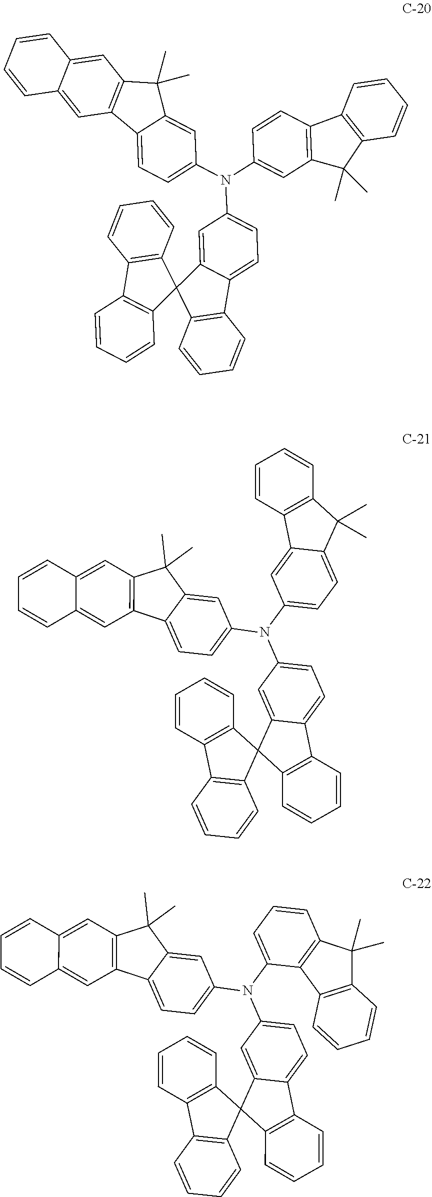 [SMILES: CC1(C)c2ccccc2-c2ccc(N(c3ccc4c(c3)C(C)(C)c3cc5ccccc5cc3-4)c3ccc4c(c3)C3(c5ccccc5-c5ccccc53)c3ccccc3-4)cc21 ; CC1(C)c2ccccc2-c2cc(N(c3ccc4c(c3)C(C)(C)c3cc5ccccc5cc3-4)c3ccc4c(c3)C3(c5ccccc5-c5ccccc53)c3ccccc3-4)ccc21 ; CC1(C)c2cc(N(c3ccc4c(c3)C3(c5ccccc5-c5ccccc53)c3ccccc3-4)c3cccc4c3-c3ccccc3C4(C)C)ccc2-c2cc3ccccc3cc21]

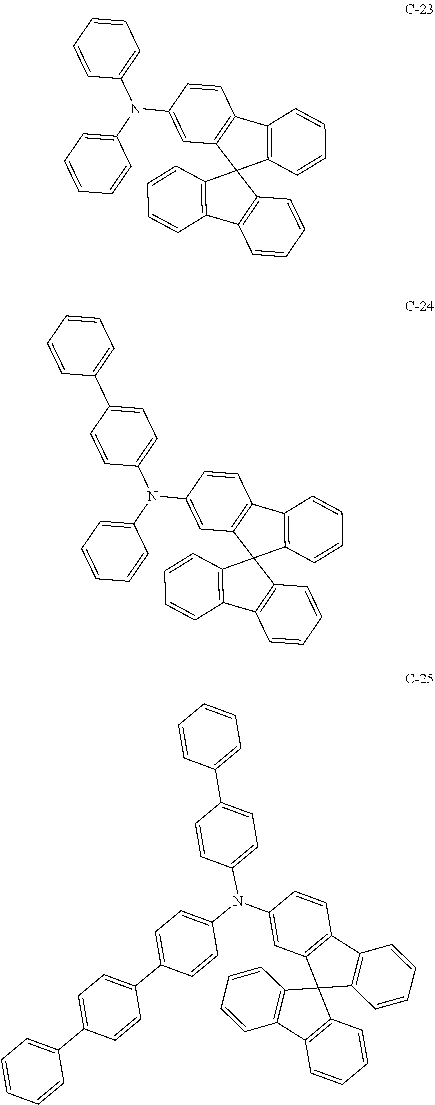 [SMILES: c1ccc(N(c2ccccc2)c2ccc3c(c2)C2(c4ccccc4-c4ccccc42)c2ccccc2-3)cc1 ; c1ccc(-c2ccc(N(c3ccccc3)c3ccc4c(c3)C3(c5ccccc5-c5ccccc53)c3ccccc3-4)cc2)cc1 ; c1ccc(-c2ccc(-c3ccc(N(c4ccc(-c5ccccc5)cc4)c4ccc5c(c4)C4(c6ccccc6-c6ccccc64)c4ccccc4-5)cc3)cc2)cc1]

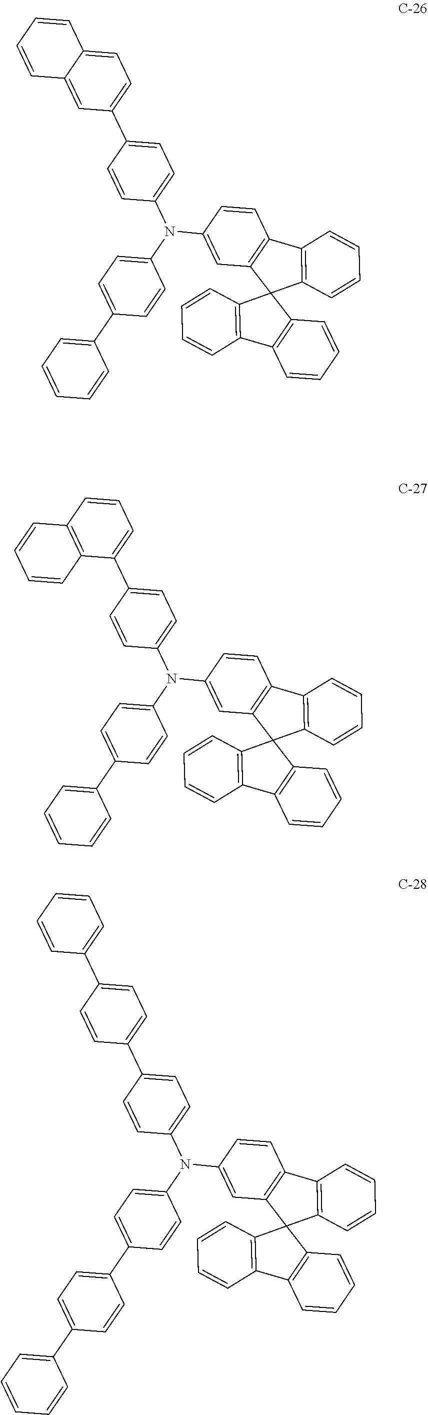 [SMILES: c1ccc(-c2ccc(N(c3ccc(-c4ccc5ccccc5c4)cc3)c3ccc4c(c3)C3(c5ccccc5-c5ccccc53)c3ccccc3-4)cc2)cc1 ; c1ccc(-c2ccc(N(c3ccc(-c4cccc5ccccc45)cc3)c3ccc4c(c3)C3(c5ccccc5-c5ccccc53)c3ccccc3-4)cc2)cc1 ; c1ccc(-c2ccc(-c3ccc(N(c4ccc(-c5ccc(-c6ccccc6)cc5)cc4)c4ccc5c(c4)C4(c6ccccc6-c6ccccc64)c4ccccc4-5)cc3)cc2)cc1]

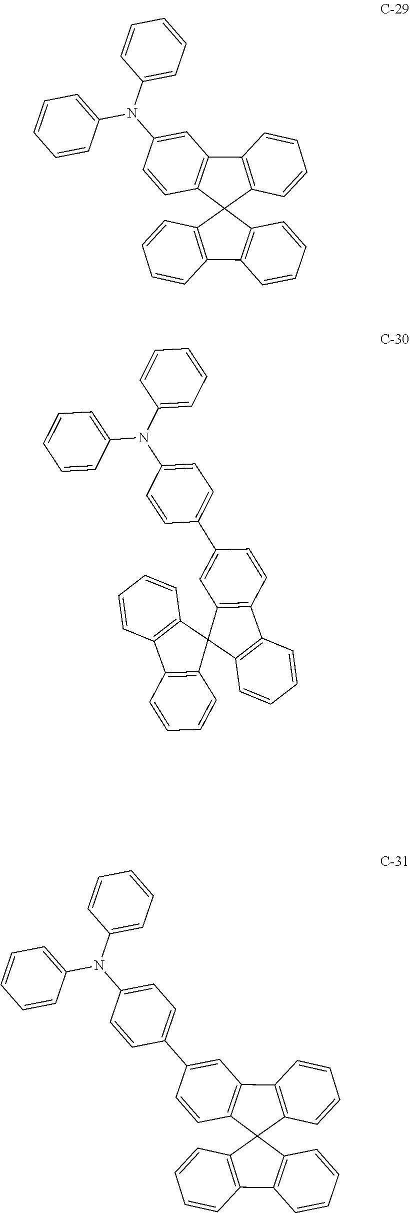 [SMILES: c1ccc(N(c2ccccc2)c2ccc3c(c2)-c2ccccc2C32c3ccccc3-c3ccccc32)cc1 ; c1ccc(N(c2ccccc2)c2ccc(-c3ccc4c(c3)C3(c5ccccc5-c5ccccc53)c3ccccc3-4)cc2)cc1 ; c1ccc(N(c2ccccc2)c2ccc(-c3ccc4c(c3)-c3ccccc3C43c4ccccc4-c4ccccc43)cc2)cc1]

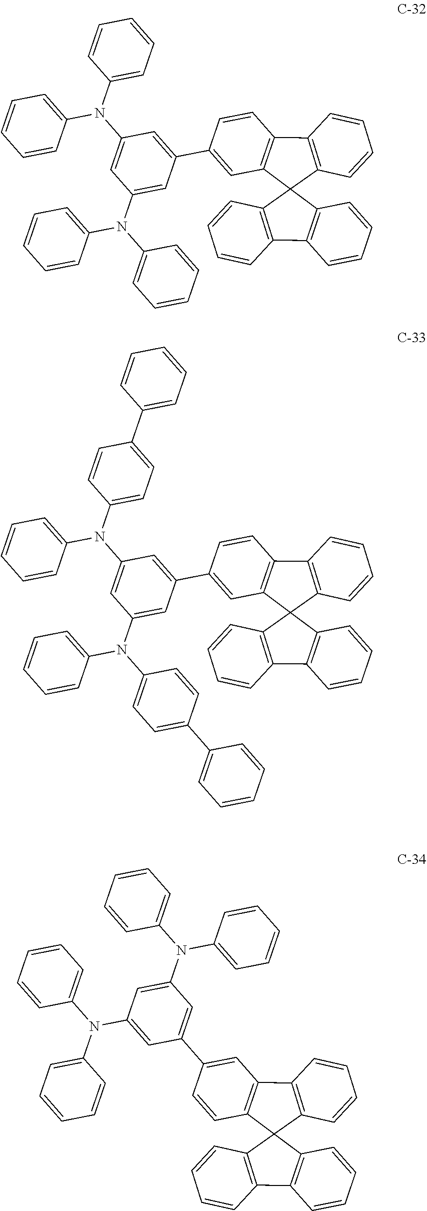

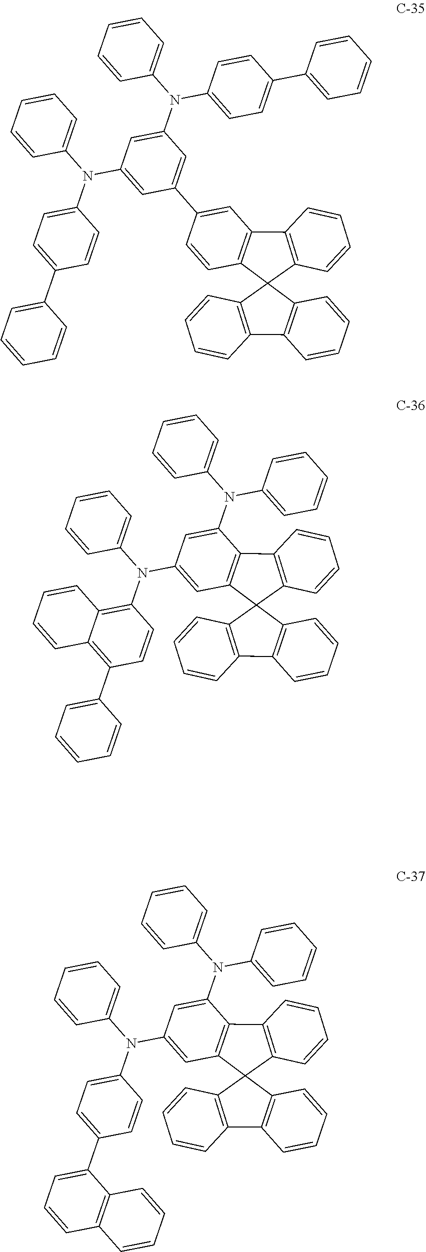 [SMILES: c1ccc(-c2ccc(N(c3ccccc3)c3cc(-c4ccc5c(c4)-c4ccccc4C54c5ccccc5-c5ccccc54)cc(N(c4ccccc4)c4ccc(-c5ccccc5)cc4)c3)cc2)cc1 ; c1ccc(-c2ccc(N(c3ccccc3)c3cc(N(c4ccccc4)c4ccccc4)c4c(c3)C3(c5ccccc5-c5ccccc53)c3ccccc3-4)c3ccccc23)cc1 ; c1ccc(N(c2ccc(-c3cccc4ccccc34)cc2)c2cc(N(c3ccccc3)c3ccccc3)c3c(c2)C2(c4ccccc4-c4ccccc42)c2ccccc2-3)cc1]

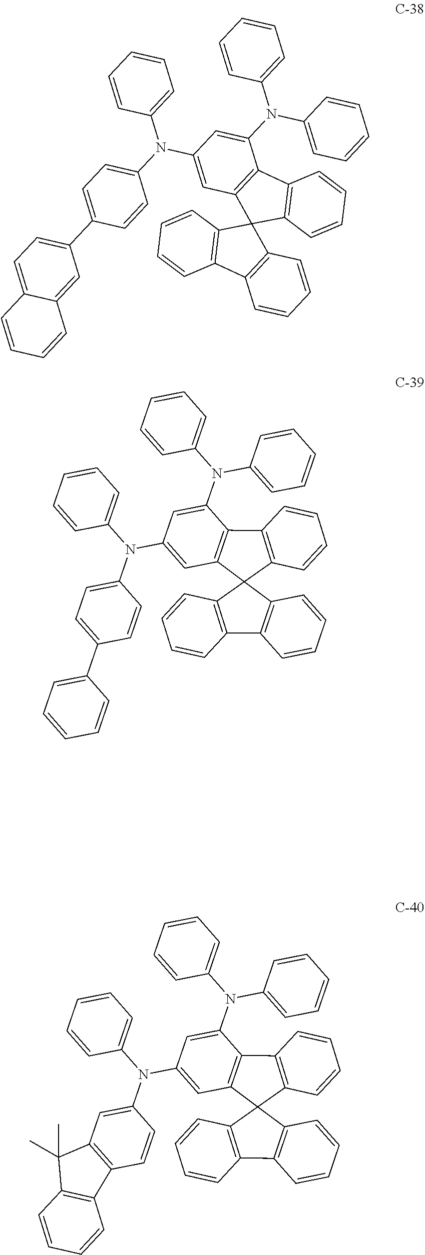 [SMILES: c1ccc(N(c2ccc(-c3ccc4ccccc4c3)cc2)c2cc(N(c3ccccc3)c3ccccc3)c3c(c2)C2(c4ccccc4-c4ccccc42)c2ccccc2-3)cc1 ; c1ccc(-c2ccc(N(c3ccccc3)c3cc(N(c4ccccc4)c4ccccc4)c4c(c3)C3(c5ccccc5-c5ccccc53)c3ccccc3-4)cc2)cc1 ; CC1(C)c2ccccc2-c2ccc(N(c3ccccc3)c3cc(N(c4ccccc4)c4ccccc4)c4c(c3)C3(c5ccccc5-c5ccccc53)c3ccccc3-4)cc21]

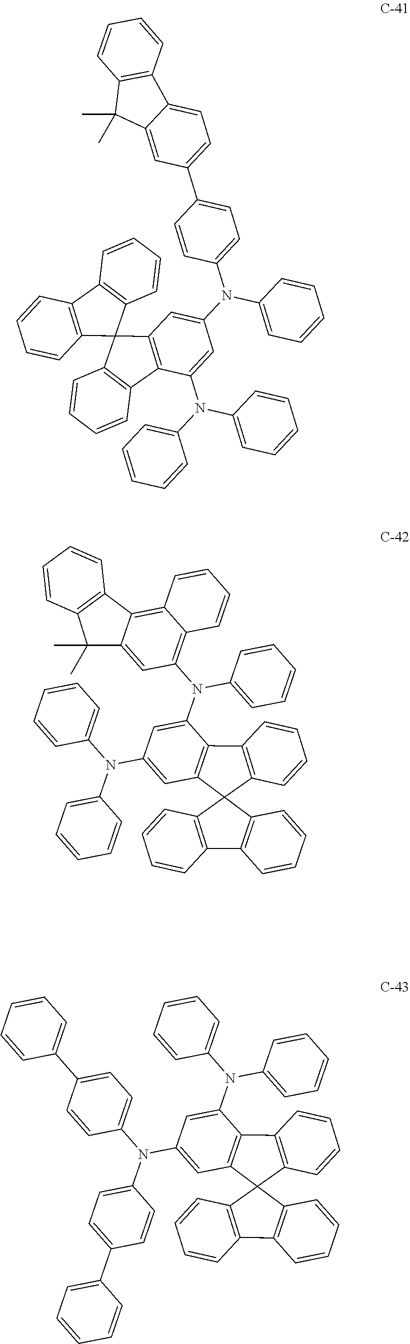 [SMILES: CC1(C)c2ccccc2-c2ccc(-c3ccc(N(c4ccccc4)c4cc(N(c5ccccc5)c5ccccc5)c5c(c4)C4(c6ccccc6-c6ccccc64)c4ccccc4-5)cc3)cc21 ; CC1(C)c2ccccc2-c2c1cc(N(c1ccccc1)c1cc(N(c3ccccc3)c3ccccc3)cc3c1-c1ccccc1C31c3ccccc3-c3ccccc31)c1ccccc21 ; c1ccc(-c2ccc(N(c3ccc(-c4ccccc4)cc3)c3cc(N(c4ccccc4)c4ccccc4)c4c(c3)C3(c5ccccc5-c5ccccc53)c3ccccc3-4)cc2)cc1]

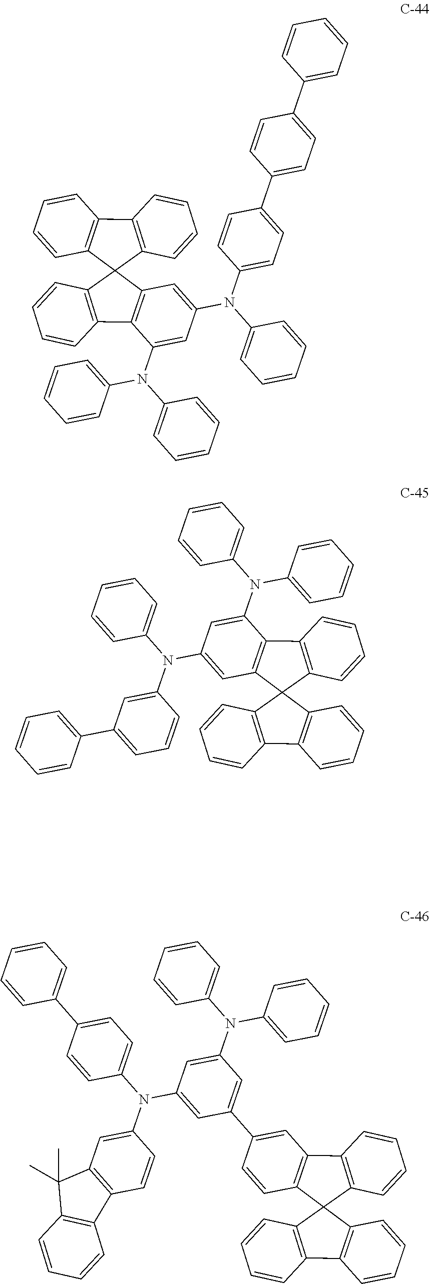 [SMILES: c1ccc(-c2ccc(-c3ccc(N(c4ccccc4)c4cc(N(c5ccccc5)c5ccccc5)c5c(c4)C4(c6ccccc6-c6ccccc64)c4ccccc4-5)cc3)cc2)cc1 ; c1ccc(-c2cccc(N(c3ccccc3)c3cc(N(c4ccccc4)c4ccccc4)c4c(c3)C3(c5ccccc5-c5ccccc53)c3ccccc3-4)c2)cc1 ; CC1(C)c2ccccc2-c2ccc(N(c3ccc(-c4ccccc4)cc3)c3cc(-c4ccc5c(c4)-c4ccccc4C54c5ccccc5-c5ccccc54)cc(N(c4ccccc4)c4ccccc4)c3)cc21]

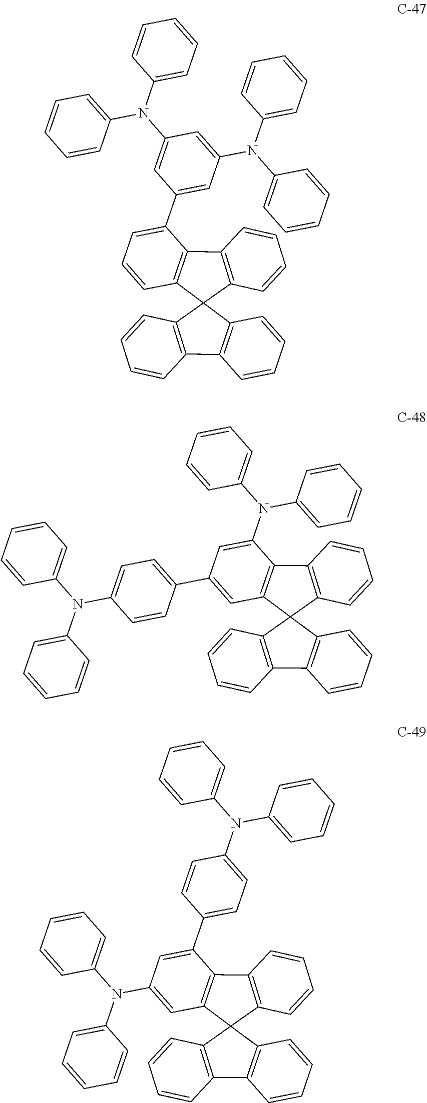 [SMILES: c1ccc(N(c2ccccc2)c2cc(-c3cccc4c3-c3ccccc3C43c4ccccc4-c4ccccc43)cc(N(c3ccccc3)c3ccccc3)c2)cc1 ; c1ccc(N(c2ccccc2)c2ccc(-c3cc(N(c4ccccc4)c4ccccc4)c4c(c3)C3(c5ccccc5-c5ccccc53)c3ccccc3-4)cc2)cc1 ; c1ccc(N(c2ccccc2)c2ccc(-c3cc(N(c4ccccc4)c4ccccc4)cc4c3-c3ccccc3C43c4ccccc4-c4ccccc43)cc2)cc1]

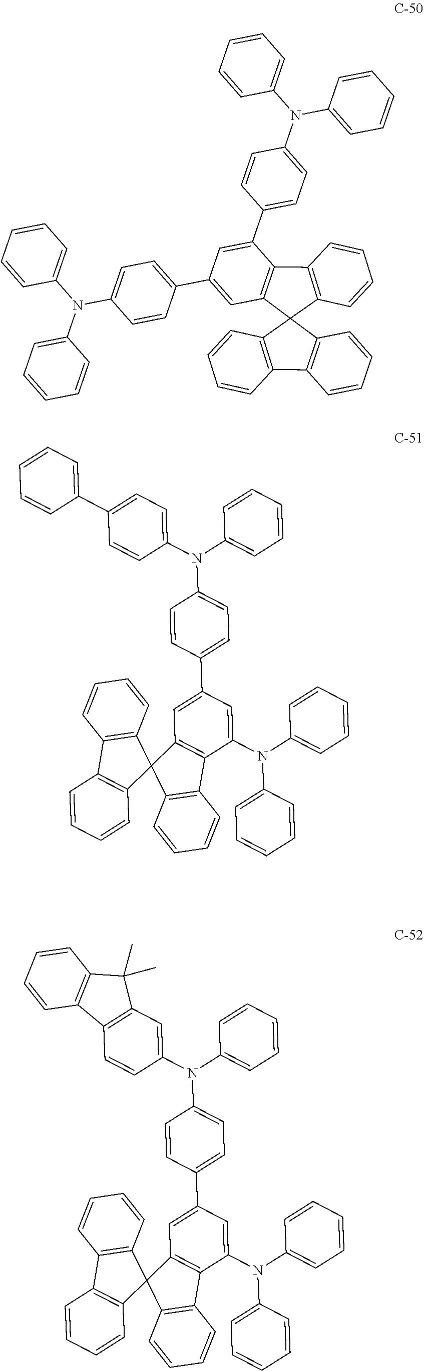 [SMILES: c1ccc(N(c2ccccc2)c2ccc(-c3cc(-c4ccc(N(c5ccccc5)c5ccccc5)cc4)c4c(c3)C3(c5ccccc5-c5ccccc53)c3ccccc3-4)cc2)cc1 ; c1ccc(-c2ccc(N(c3ccccc3)c3ccc(-c4cc(N(c5ccccc5)c5ccccc5)c5c(c4)C4(c6ccccc6-c6ccccc64)c4ccccc4-5)cc3)cc2)cc1 ; CC1(C)c2ccccc2-c2ccc(N(c3ccccc3)c3ccc(-c4cc(N(c5ccccc5)c5ccccc5)c5c(c4)C4(c6ccccc6-c6ccccc64)c4ccccc4-5)cc3)cc21]

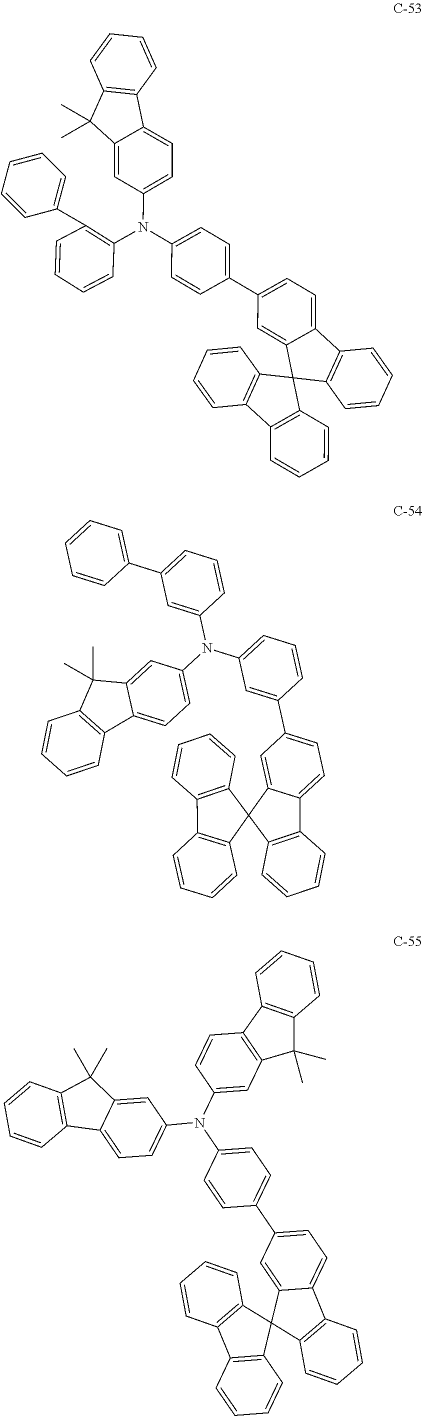 [SMILES: CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21 ; CC1(C)c2ccccc2-c2ccc(N(c3cccc(-c4ccccc4)c3)c3cccc(-c4ccc5c(c4)C4(c6ccccc6-c6ccccc64)c4ccccc4-5)c3)cc21 ; CC1(C)c2ccccc2-c2ccc(N(c3ccc(-c4ccc5c(c4)C4(c6ccccc6-c6ccccc64)c4ccccc4-5)cc3)c3ccc4c(c3)C(C)(C)c3ccccc3-4)cc21]

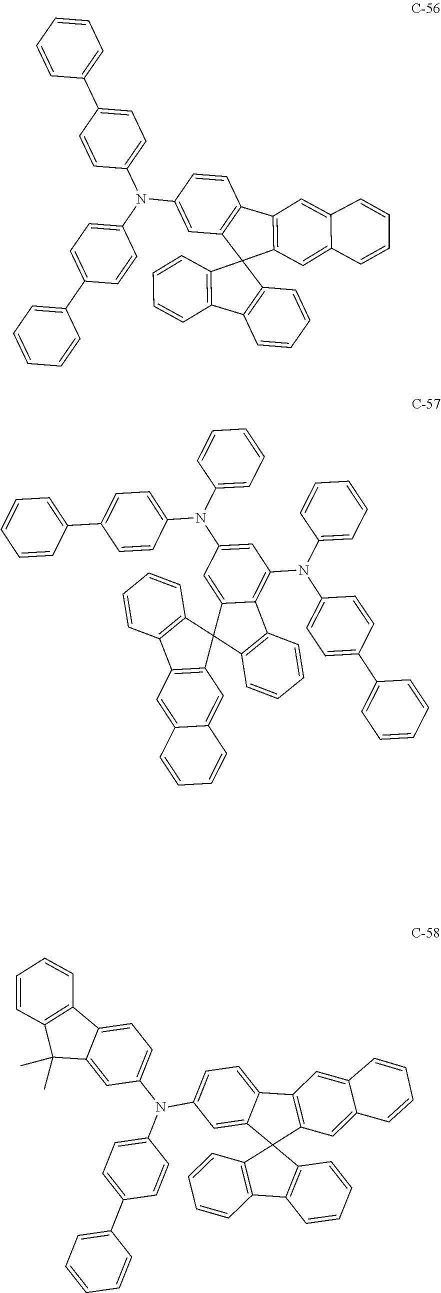 [SMILES: c1ccc(-c2ccc(N(c3ccc(-c4ccccc4)cc3)c3ccc4c(c3)C3(c5ccccc5-c5ccccc53)c3cc5ccccc5cc3-4)cc2)cc1 ; c1ccc(-c2ccc(N(c3ccccc3)c3cc(N(c4ccccc4)c4ccc(-c5ccccc5)cc4)c4c(c3)C3(c5ccccc5-c5cc6ccccc6cc53)c3ccccc3-4)cc2)cc1 ; CC1(C)c2ccccc2-c2ccc(N(c3ccc(-c4ccccc4)cc3)c3ccc4c(c3)C3(c5ccccc5-c5ccccc53)c3cc5ccccc5cc3-4)cc21]

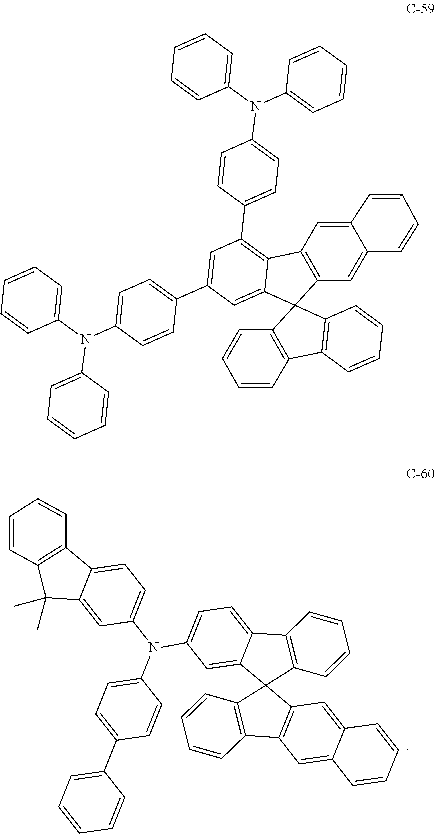 [SMILES: c1ccc(N(c2ccccc2)c2ccc(-c3cc(-c4ccc(N(c5ccccc5)c5ccccc5)cc4)c4c(c3)C3(c5ccccc5-c5ccccc53)c3cc5ccccc5cc3-4)cc2)cc1 ; CC1(C)c2ccccc2-c2ccc(N(c3ccc(-c4ccccc4)cc3)c3ccc4c(c3)C3(c5ccccc5-4)c4ccccc4-c4cc5ccccc5cc43)cc21]

8. The organic electroluminescence device according to claim 1, wherein the compound represented by Formula 2 is selected from the group consisting of: 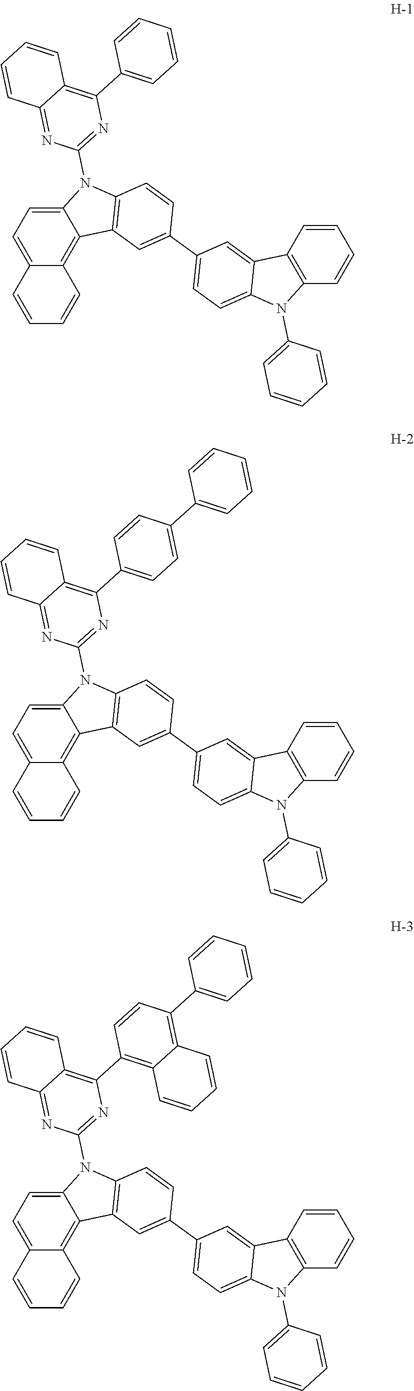 [SMILES: c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-c3nc(-n4c5ccc(-c6ccc7c(c6)c6ccccc6n7-c6ccccc6)cc5c5c6ccccc6ccc54)nc4ccccc34)cc2)cc1 ; c1ccc(-c2ccc(-c3nc(-n4c5ccc(-c6ccc7c(c6)c6ccccc6n7-c6ccccc6)cc5c5c6ccccc6ccc54)nc4ccccc34)c3ccccc23)cc1] 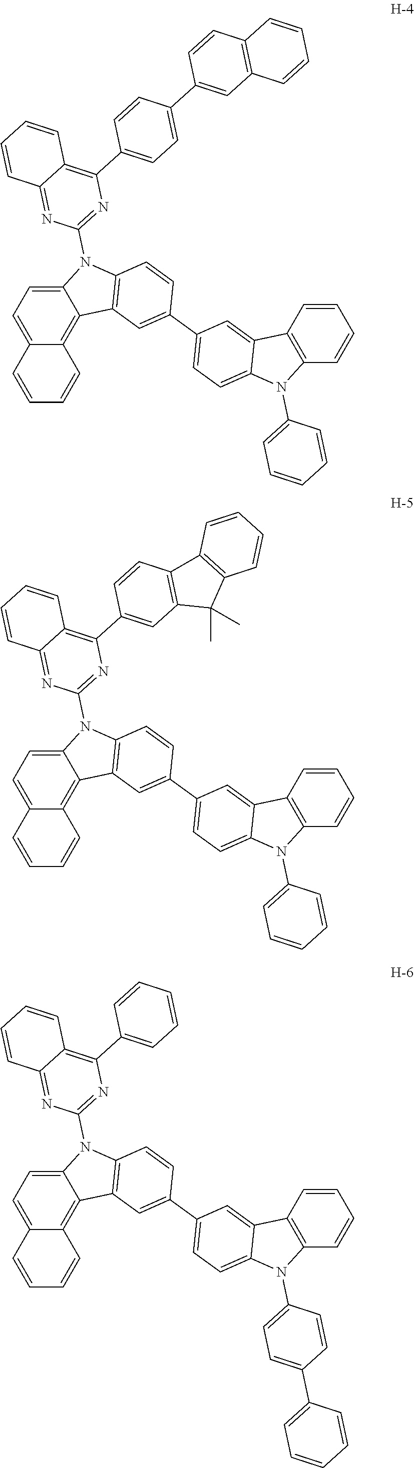 [SMILES: c1ccc(-n2c3ccccc3c3cc(-c4ccc5c(c4)c4c6ccccc6ccc4n5-c4nc(-c5ccc(-c6ccc7ccccc7c6)cc5)c5ccccc5n4)ccc32)cc1 ; CC1(C)c2ccccc2-c2ccc(-c3nc(-n4c5ccc(-c6ccc7c(c6)c6ccccc6n7-c6ccccc6)cc5c5c6ccccc6ccc54)nc4ccccc34)cc21 ; c1ccc(-c2ccc(-n3c4ccccc4c4cc(-c5ccc6c(c5)c5c7ccccc7ccc5n6-c5nc(-c6ccccc6)c6ccccc6n5)ccc43)cc2)cc1] 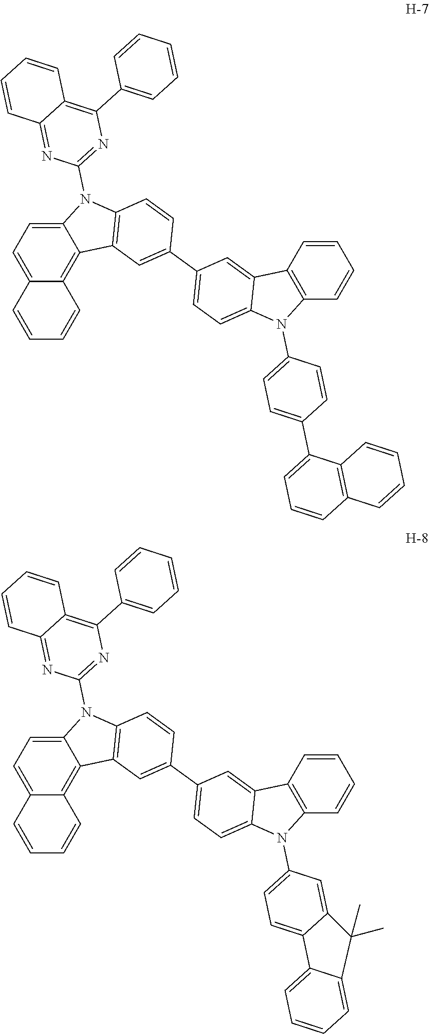 [SMILES: c1ccc(-c2nc(-n3c4ccc(-c5ccc6c(c5)c5ccccc5n6-c5ccc(-c6cccc7ccccc67)cc5)cc4c4c5ccccc5ccc43)nc3ccccc23)cc1 ; CC1(C)c2ccccc2-c2ccc(-n3c4ccccc4c4cc(-c5ccc6c(c5)c5c7ccccc7ccc5n6-c5nc(-c6ccccc6)c6ccccc6n5)ccc43)cc21] 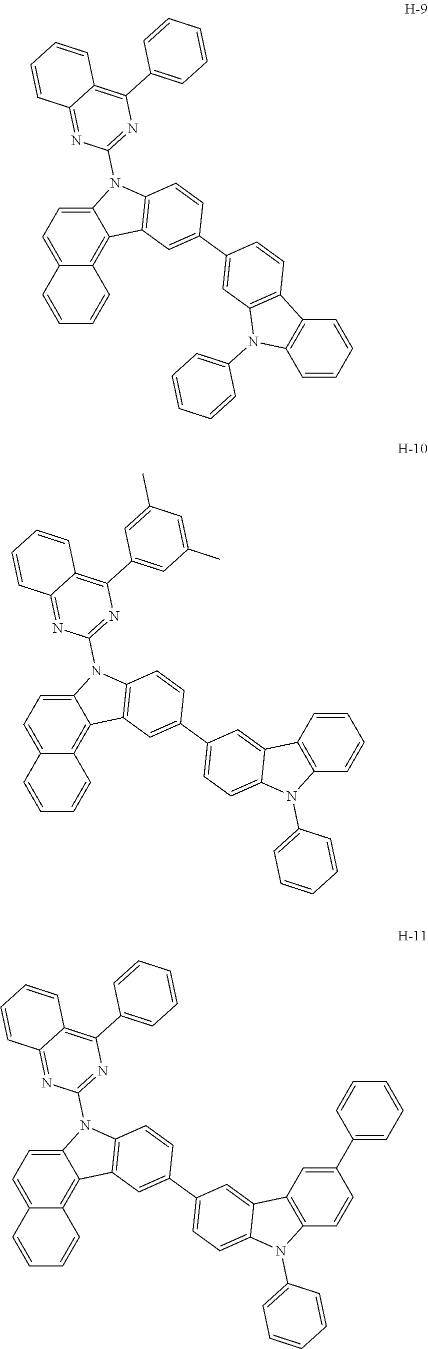 [SMILES: c1ccc(-c2nc(-n3c4ccc(-c5ccc6c7ccccc7n(-c7ccccc7)c6c5)cc4c4c5ccccc5ccc43)nc3ccccc23)cc1 ; Cc1cc(C)cc(-c2nc(-n3c4ccc(-c5ccc6c(c5)c5ccccc5n6-c5ccccc5)cc4c4c5ccccc5ccc43)nc3ccccc23)c1 ; c1ccc(-c2ccc3c(c2)c2cc(-c4ccc5c(c4)c4c6ccccc6ccc4n5-c4nc(-c5ccccc5)c5ccccc5n4)ccc2n3-c2ccccc2)cc1] 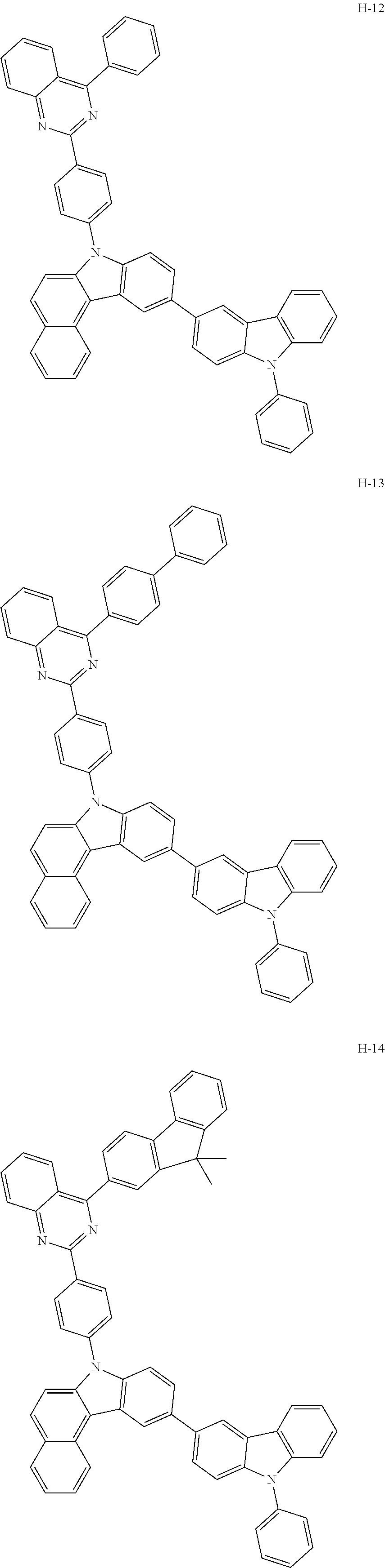 [SMILES: c1ccc(-c2nc(-c3ccc(-n4c5ccc(-c6ccc7c(c6)c6ccccc6n7-c6ccccc6)cc5c5c6ccccc6ccc54)cc3)nc3ccccc23)cc1 ; c1ccc(-c2ccc(-c3nc(-c4ccc(-n5c6ccc(-c7ccc8c(c7)c7ccccc7n8-c7ccccc7)cc6c6c7ccccc7ccc65)cc4)nc4ccccc34)cc2)cc1 ; CC1(C)c2ccccc2-c2ccc(-c3nc(-c4ccc(-n5c6ccc(-c7ccc8c(c7)c7ccccc7n8-c7ccccc7)cc6c6c7ccccc7ccc65)cc4)nc4ccccc34)cc21] 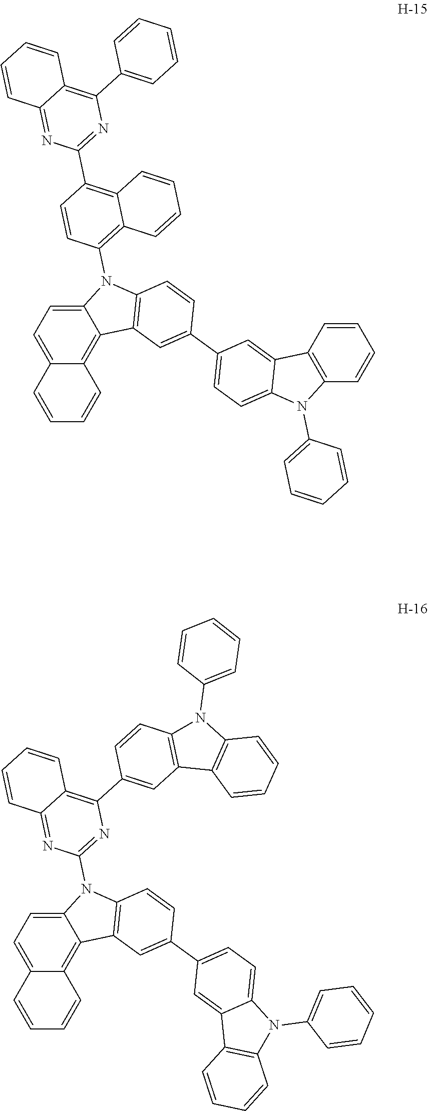 [SMILES: c1ccc(-c2nc(-c3ccc(-n4c5ccc(-c6ccc7c(c6)c6ccccc6n7-c6ccccc6)cc5c5c6ccccc6ccc54)c4ccccc34)nc3ccccc23)cc1 ; c1ccc(-n2c3ccccc3c3cc(-c4ccc5c(c4)c4c6ccccc6ccc4n5-c4nc(-c5ccc6c(c5)c5ccccc5n6-c5ccccc5)c5ccccc5n4)ccc32)cc1] 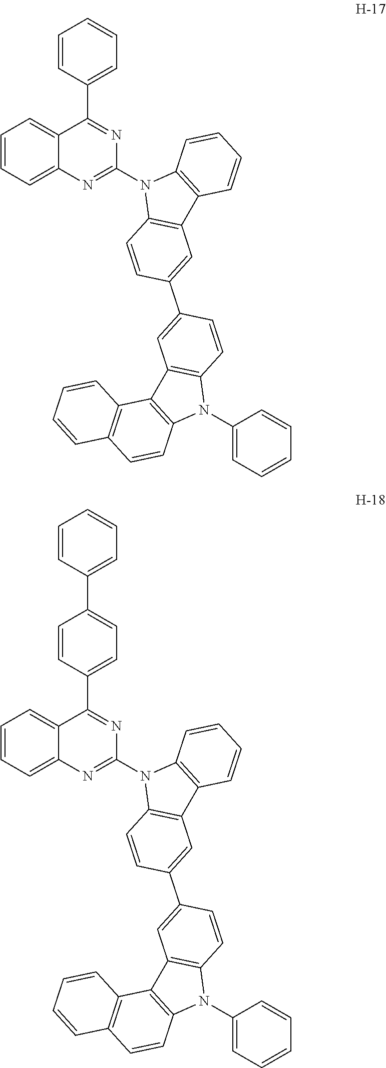 [SMILES: c1ccc(-c2nc(-n3c4ccccc4c4cc(-c5ccc6c(c5)c5c7ccccc7ccc5n6-c5ccccc5)ccc43)nc3ccccc23)cc1 ; c1ccc(-c2ccc(-c3nc(-n4c5ccccc5c5cc(-c6ccc7c(c6)c6c8ccccc8ccc6n7-c6ccccc6)ccc54)nc4ccccc34)cc2)cc1] 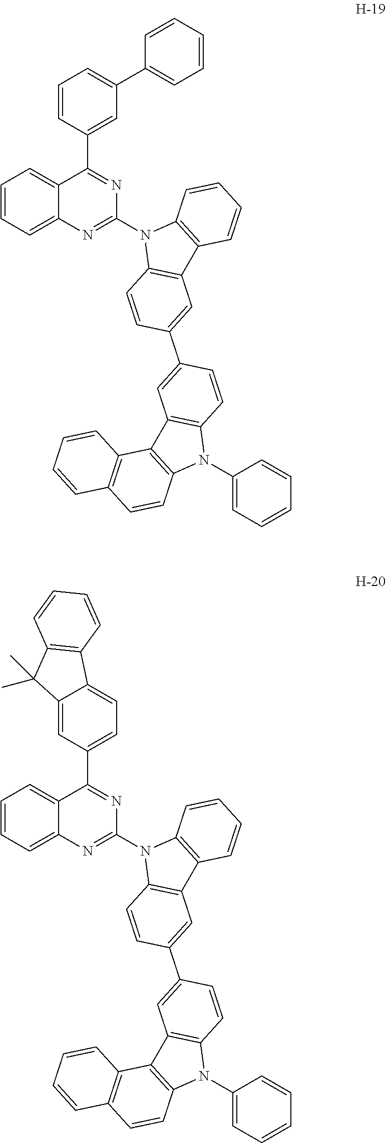 [SMILES: c1ccc(-c2cccc(-c3nc(-n4c5ccccc5c5cc(-c6ccc7c(c6)c6c8ccccc8ccc6n7-c6ccccc6)ccc54)nc4ccccc34)c2)cc1 ; CC1(C)c2ccccc2-c2ccc(-c3nc(-n4c5ccccc5c5cc(-c6ccc7c(c6)c6c8ccccc8ccc6n7-c6ccccc6)ccc54)nc4ccccc34)cc21] 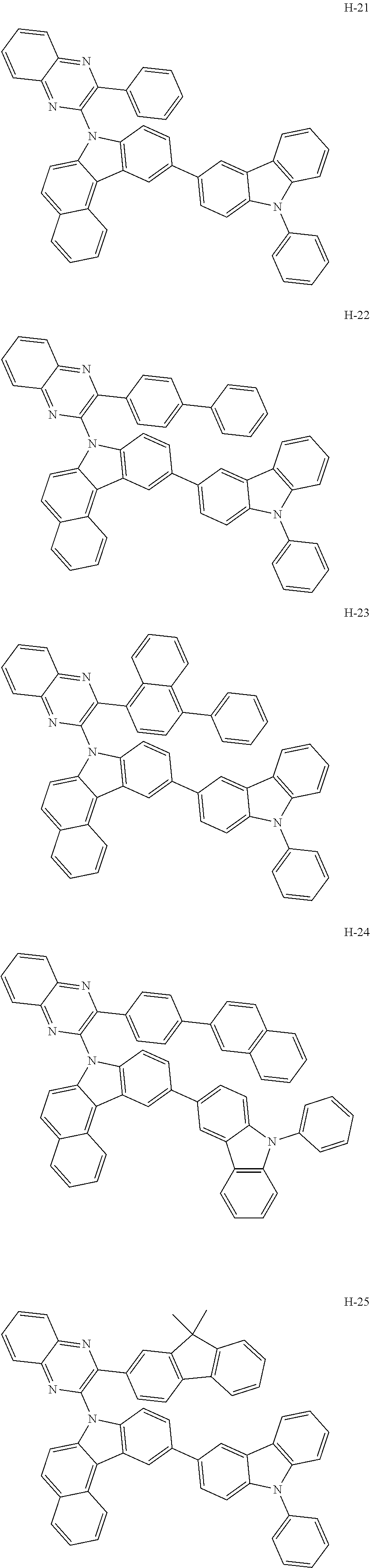 [SMILES: c1ccc(-c2nc3ccccc3nc2-n2c3ccc(-c4ccc5c(c4)c4ccccc4n5-c4ccccc4)cc3c3c4ccccc4ccc32)cc1 ; c1ccc(-c2ccc(-c3nc4ccccc4nc3-n3c4ccc(-c5ccc6c(c5)c5ccccc5n6-c5ccccc5)cc4c4c5ccccc5ccc43)cc2)cc1 ; c1ccc(-c2ccc(-c3nc4ccccc4nc3-n3c4ccc(-c5ccc6c(c5)c5ccccc5n6-c5ccccc5)cc4c4c5ccccc5ccc43)c3ccccc23)cc1 ; c1ccc(-n2c3ccccc3c3cc(-c4ccc5c(c4)c4c6ccccc6ccc4n5-c4nc5ccccc5nc4-c4ccc(-c5ccc6ccccc6c5)cc4)ccc32)cc1 ; CC1(C)c2ccccc2-c2ccc(-c3nc4ccccc4nc3-n3c4ccc(-c5ccc6c(c5)c5ccccc5n6-c5ccccc5)cc4c4c5ccccc5ccc43)cc21]
